(19) United States
(12) Patent Application Publication
(10) Pub. No.: US 2025/0314639 A1
(43) Pub. Date: Oct. 9, 2025

(54) SYSTEMS AND METHODS FOR HIGH THROUGHPUT SINGLE MOLECULE TRACKING IN LIVING CELLS

(71) Applicant: EIKON THERAPEUTICS, INC., Millbrae, CA (US)

(72) Inventors: Daniel ANDERSON, Millbrae, CA (US); Jaclyn HO, Millbrae, CA (US); Russell BERMAN, Millbrae, CA (US); Brian MARGOLIN, Millbrae, CA (US); David MCSWIGGEN, Millbrae, CA (US)

(73) Assignee: EIKON THERAPEUTICS, INC., Millbrae, CA (US)

(21) Appl. No.: 19/244,317

(22) Filed: Jun. 20, 2025

Related U.S. Application Data
(63) Continuation of application No. PCT/US2023/085590, filed on Dec. 21, 2023.
(60) Provisional application No. 63/476,950, filed on Dec. 22, 2022.
(60) Provisional application No. 63/476,941, filed on Dec. 22, 2022.

(51) Int. Cl. G01N 33/50; G01N 21/64; G01N 33/58; G02B 21/16; G02B 21/36

(52) U.S. Cl. CPC G01N 33/5035; G01N 21/6402; G01N 21/6428; G01N 21/6458; G01N 33/582; G02B 21/16; G02B 21/365; G01N 2021/6439

(57) ABSTRACT

High Throughput Single Molecule Tracking (htSMT) systems and methods are described wherein the htSMT workflows are adapted to characterize both known and novel pathway contributions to interaction networks in live cells, such as protein signaling interaction networks.

### CROSS-REFERENCE TO RELATED APPLICATIONS

This application is a continuation of International Patent Application No. PCT/US2023/085590, filed Dec. 21, 2023, which claims priority to U.S. Provisional Application No. 63/476,950, filed Dec. 22, 2022, and U.S. Provisional Application No. 63/476,941, filed Dec. 22, 2022, the contents of each of which are incorporated herein by reference herein in their entirety.

### TECHNICAL FIELD

The subject matter described herein relates to a platform to track single molecules within complex systems.

### BACKGROUND

The movement of proteins within the crowded environment of living cells are profoundly influenced by interactions with their surroundings. Single molecule tracking (SMT) is one method for capturing protein movement as a reporter of activity. In SMT, a fluorescent protein of interest is imaged at high spatiotemporal resolution to track its movement in a complex system, e.g., a live cell. The information embedded in these tracks has been used to investigate diverse cellular phenomena including protein-protein interactions, e.g., interactions mediating signal transduction, inter-organelle communication, nuclear organization, and transcription regulation. The application of SMT techniques has been limited in scale, however, and therefore mainly used to address specific mechanistic hypotheses. For example, SMT has not been adapted to a throughput setting that would enable systems-level screening or drug discovery.

### SUMMARY OF THE INVENTION

In a first aspect, the present disclosure is directed to a method of determining the rate of emergence of a biological interaction between a compound and a target fluorescent protein in a live cell comprising: (a) contacting a sample comprising a population of live cells with the compound, wherein the live cells comprise the target fluorescent protein; (b) tracking the movement of a plurality of individual target fluorescent proteins in a plurality of live cells in the sample at a plurality of time points, wherein said tracking comprises: (i) illuminating a field of view in a sample plane disposed within the sample with a light beam to cause fluorescence by a subset of the target fluorescent proteins in the live cells; and (ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and (c) determining a change in the movement of the target fluorescent protein in the presence of the compound, wherein the rate at which changes in the movement of the target fluorescent protein emerge is determined by comparing the changes in movement of the target fluorescent protein at the plurality of time points.

In an interrelated aspect the present disclosure is directed to a method of determining the rate of emergence of a biological interaction between a compound and a target fluorescent protein in a live cell comprising: (a) contacting a sample comprising a population of live cells with the compound, wherein the live cells comprise the target fluorescent protein; (b) tracking the movement of a plurality of individual target fluorescent proteins in a plurality of live cells in the sample at a plurality of time points, wherein said tracking comprises: (i) illuminating a field of view in a sample plane disposed within the sample with a light beam to cause fluorescence by a subset of the target fluorescent proteins in the live cells, wherein the subset of the target fluorescent produces 100-100,000 molecular trajectories in a single detected field of view; and (ii) detecting the fluorescence from a plurality of the target fluorescent proteins in the detected field of view of the sample plane via a detector device, wherein the detected field of view has a size of about 50 μm to less than 100 μm in a first dimension by about 50 μm to less than 100 μm in a second dimension; and (c) determining a change in the movement of the target fluorescent protein in the presence of the compound, wherein the rate at which changes in the movement of the target fluorescent protein emerge is determined by comparing the changes in movement of the target fluorescent protein at the plurality of time points.

In an interrelated aspect the present disclosure is directed to a method determining the rate of emergence of a biological interaction between a compound and a target fluorescent protein in a live cell comprising: (a) contacting a sample comprising a population of live cells with the compound, wherein the live cells comprise the target fluorescent protein; (b) tracking the movement of a plurality of individual target fluorescent proteins in a plurality of live cells in the sample at a plurality of time points, wherein said tracking comprises: (i) illuminating a field of view in a sample plane disposed within the sample with a light beam to cause fluorescence by a subset of the target fluorescent proteins in the live cells; and (ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and (c) determining a change in the movement of target fluorescent proteins upon the addition of the compound, wherein the average change in movement of the target fluorescent proteins in the presence of the compound is at least 1%, at least 5%, at least 10% relative to the change observed in the absence of the compound, wherein the rate at which changes in the movement of the target fluorescent protein emerge is determined by comparing the changes in movement of the target fluorescent protein at the plurality of time points.

In an interrelated aspect the present disclosure is directed to a method of determining the rate of emergence of a biological interaction between a compound and a target fluorescent protein in a live cell comprising: (a) contacting a sample comprising a population of live cells with the compound, wherein the live cells comprise the target fluorescent protein; (b) tracking the movement of a plurality of individual target fluorescent proteins in a plurality of live cells in the sample at a plurality of time points, wherein said tracking comprises: (i) illuminating a field of view in a sample plane disposed within the sample with a light beam to cause fluorescence by a subset of the target fluorescent proteins in the live cells; (ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and (iii) detecting the fluorescence from a plurality of the target fluorescent proteins in the detected field of view of the sample plane at a rate of >100 detected FOVs per day, >10,000 FOVs per day, or >100,000 detected FOVs per day; and (c) determining a change in the movement of target fluorescent proteins in the presence of the compound, wherein the rate at which changes in the movement of the target fluorescent protein emerge is determined by comparing the changes in movement of the target fluorescent protein at the plurality of time points.

In an interrelated aspect the present disclosure is directed to a method of determining the rate of emergence of a biological interaction between a compound and a target fluorescent protein in a live cell comprising: (a) contacting a sample comprising a population of live cells with the compound, wherein the live cells comprise the target fluorescent protein; (b) tracking the movement of a plurality of individual target fluorescent proteins in a plurality of live cells in the sample at a plurality of time points, wherein said tracking comprises: (i) illuminating a field of view in a sample plane disposed within the sample with a light beam to cause fluorescence by a subset of the target fluorescent proteins in the live cells; and (ii) detecting the fluorescence from a plurality of the target fluorescent proteins in the detected field of view of the sample plane via a detector device, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; and (c) determining a change in the movement of the target fluorescent protein in the presence of the compound, wherein the rate at which changes in the movement of the target fluorescent protein emerge is determined by comparing the changes in movement of the target fluorescent protein at the plurality of time points.

In certain instances of the above-described aspects, the change in movement detected is an increase in immobile trajectories indicating an increase in bound (f_{bound}) target fluorescent protein. In certain instances of the above-described aspects, the change in movement detected is a change in: (a) the median of the jump length distribution; (b) 3^{rd} quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; and/or (j) state occupation via inference. In certain instances of the above-described aspects, the target fluorescent protein interacts in a larger molecular assembly. In certain instances of the above-described aspects, the target fluorescent protein is a ligand. In certain instances of the above-described aspects, the target fluorescent protein is a receptor. In certain instances of the above-described aspects, the biological interaction is a direct interaction. In certain instances of the above-described aspects, the direct interaction comprises binding of the compound to the target fluorescent protein. In certain instances of the above-described aspects, the biological interaction is an indirect interaction. In certain instances of the above-described aspects, the indirect interaction comprises the compound agonizing or antagonizing a larger molecular assembly comprising the target fluorescent protein.

In an interrelated aspect the present disclosure is directed to a method of determining a dose of a compound, wherein said compound induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; and (b) tracking the movement of individual target fluorescent proteins in a plurality of live cells of a sample at a plurality of time points, wherein said tracking comprises: (i) illuminating a field of view in a sample plane disposed within the sample with a light beam to cause fluorescence by at least a subset of the target fluorescent proteins in the live cells; (ii) detecting the fluorescence from one or more of the target fluorescent proteins in the sample plane via a detector device, wherein the detected field of view has a size of about 50 μm to less than 100 μm in a first dimension by about 50 μm to less than 100 μm in a second dimension; and (c) determining the rate at which changes in the movement of the target fluorescent protein occur in the presence of the compound; and (d) repeating steps (b)-(c) for each of the plurality of samples across the range of compound concentrations, wherein the rate at which changes in the movement of the target fluorescent protein emerge is determined by comparing the changes in movement of the target fluorescent protein at the plurality of time points; and wherein the dose is determined by said rate.

In an interrelated aspect the present disclosure is directed to a method of determining a dose of a compound that induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; and (b) tracking the movement of individual target fluorescent proteins in a plurality of live cells of a sample at a plurality of time points, wherein said tracking comprises: (i) illuminating a field of view in a sample plane disposed within the sample with a light beam to cause fluorescence by at least a subset of the target fluorescent proteins in the live cells, wherein the subset of the target fluorescent proteins produces 100-100,000 molecular trajectories in a single detected field of view; (ii) detecting the fluorescence from one or more of the target fluorescent proteins in the detected field of view of the sample plane via a detector device, wherein the detected field of view has a size of about 50 μm to less than 100 μm in a first dimension by about 50 μm to less than 100 μm in a second dimension; and (c) determining the rate at which changes in the movement of the target fluorescent protein occur in the presence of the compound; and (d) repeating steps (b)-(c) for each of the plurality of samples across the range of compound concentrations, wherein the rate at which changes in the movement of the target fluorescent protein emerge is determined by comparing the changes in movement of the target fluorescent protein at the plurality of time points; and wherein the dose is determined by said rate.

In an interrelated aspect the present disclosure is directed to a method of determining a dose response of a compound that induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; and (b) tracking the movement of individual target fluorescent proteins in a plurality of live cells of a sample at a plurality of time points, wherein said tracking comprises: (i) illuminating a field of view in a sample plane disposed within the sample with a light beam to cause fluorescence by at least a subset of the target fluorescent proteins in the live cells; (ii) detecting the fluorescence from one or more of the target fluorescent proteins in a detected field of view of the sample plane via a detector device, wherein the detected field of view has a size of about 50 μm to less than 100 μm in a first dimension by about 50 μm to less than 100 μm in a second dimension; and (c) determining the rate at which changes in the movement of the target fluorescent protein occur in the presence of the compound, wherein the average change in movement of the target fluorescent protein in the presence of the compound is at least 1%, at least 5%, at least 10%, relative to the change observed in the absence of the compound; and (d) repeating steps (b)-(c) for each of the plurality of samples across the range of compound concentrations, wherein the rate at which changes in the movement of the target fluorescent protein emerge is determined by comparing the changes in movement of the target fluorescent protein at the plurality of time points; and wherein the dose is determined by said rate.

In an interrelated aspect the present disclosure is directed to a method of determining a dose response of a compound that induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; and (b) tracking the movement of individual target fluorescent proteins in a plurality of live cells of a sample at a plurality of time points, wherein said tracking comprises: (i) illuminating a field of view in a sample plane disposed within the sample with a light beam to cause fluorescence by at least a subset of the target fluorescent proteins in the cells; (ii) detecting the fluorescence from one or more of the target fluorescent proteins in a detected field of view of the sample plane via a detector device, wherein the detected field of view has a size of about 50 μm to less than 100 μm in a first dimension by about 50 μm to less than 100 μm in a second dimension; and iii) wherein said tracking comprises detecting the fluorescence from a plurality of the target fluorescent proteins in the detected field of view of the sample plane at a rate of >100 detected FOVs per day, >10,000 detected FOVs per day, or >100,000 detected FOVs per day; and (c) determining the rate at which changes in the movement of the target fluorescent protein occur in the presence of the compound; and (d) repeating steps (b)-(c) for each of the plurality of samples across the range of compound concentrations, wherein the rate at which changes in the movement of the target fluorescent protein emerge is determined by comparing the changes in movement of the target fluorescent protein at the plurality of time points; and wherein the dose is determined by said rate.

In an interrelated aspect the present disclosure is directed to a method of determining a dose of a compound, wherein said compound induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; and (b) tracking the movement of individual target fluorescent proteins in a plurality of live cells of a sample at a plurality of time points, wherein said tracking comprises: (i) illuminating a field of view in a sample plane disposed within the sample with a light beam to cause fluorescence by at least a subset of the target fluorescent proteins in the live cells; (ii) detecting the fluorescence from one or more of the target fluorescent proteins in the sample plane via a detector device, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; and (c) determining the rate at which changes in the movement of the target fluorescent protein occur in the presence of the compound; and (d) repeating steps (b)-(c) for each of the plurality of samples across the range of compound concentrations, wherein the rate at which changes in the movement of the target fluorescent protein emerge is determined by comparing the changes in movement of the target fluorescent protein at the plurality of time points; and wherein the dose is determined by said rate.

In certain instances of the above-described aspects, the change in movement detected is an increase in immobile trajectories indicating an increase in bound (f_{bound}) target fluorescent protein. In certain instances of the above-described aspects, the change in movement detected is a change in: (a) the median of the jump length distribution; (b) 3^{rd} quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; and/or (j) state occupation via inference. In certain instances of the above-described aspects, the target fluorescent protein interacts in a larger molecular assembly. In certain instances of the above-described aspects, the target fluorescent protein is a ligand. In certain instances of the above-described aspects, the target fluorescent protein is a receptor. In certain instances of the above-described aspects, the biological interaction is a direct interaction. In certain instances of the above-described aspects, the direct interaction comprises binding of the compound to the target fluorescent protein. In certain instances of the above-described aspects, the biological interaction is an indirect interaction. In certain instances of the above-described aspects, the indirect interaction comprises the compound agonizing or antagonizing a larger molecular assembly comprising the target fluorescent protein.

In an interrelated aspect the present disclosure is directed to a microscopy system configured to detect the rate of emergence of a biological interaction between a compound and a target fluorescent protein in a live cell comprising: (a) a stage for supporting a sample, wherein the sample comprises a population of live cells, and where the live cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a subset of the target fluorescent proteins in the sample are disposed in a detected field of view of the sample plane and wherein the detected field of view has a size of about 50 μm to less than 100 μm in a first dimension by about 50 μm to less than 100 μm in a second dimension; (e) a detector device for monitoring the light-based response from the target fluorescent proteins in the presence of the compound at a plurality of time points; (e) a memory; and (f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

In an interrelated aspect the present disclosure is directed to a microscopy system configured to detect the rate of emergence of a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a subset of the target fluorescent proteins in the sample are disposed in a detected field of view in the sample plane, wherein the subset of the target fluorescent proteins produces 100-100,000 molecular trajectories in a single detected field of view and wherein the detected field of view has a size of about 50 μm to less than 100 μm in a first dimension by about 50 μm to less than 100 μm in a second dimension; (d) a detector device for monitoring the light-based response from the target fluorescent proteins in the presence of the compound at a plurality of time points; (e) a memory; and (f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

In an interrelated aspect the present disclosure is directed to a microscopy system configured to detect the rate of emergence of a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a subset of the target fluorescent proteins in the sample are disposed in a detected field of view of the sample plane and wherein the detected field of view has a size of about 50 μm to less than 100 μm in a first dimension by about 50 μm to less than 100 μm in a second dimension; (d) a detector device for monitoring the light-based response from the target fluorescent proteins in the presence of the compound at a plurality of time points, wherein the average change in movement of the target fluorescent protein in the presence of the of the compound is at least 2%, at least 5%, at least 10%, relative to the change observed in the absence of the compound; (e) a memory; and (f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

In an interrelated aspect the present disclosure is directed to a microscopy system configured to detect the rate of emergence of a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a plurality of the target fluorescent proteins in the sample are disposed in a detected field of view of the sample plane and wherein the detected field of view has a size of about 50 μm to less than 100 μm in a first dimension by about 50 μm to less than 100 μm in a second dimension; (d) a detector device for monitoring the light-based response from the target fluorescent proteins in the presence of the compound at a plurality of time points, wherein said monitoring comprises detecting the fluorescence from a plurality of the target fluorescent proteins in the detected field of view of the sample plane at a rate of >100 detected FOVs per day, >10,000 detected FOVs per day, >100,000 detected FOVs per day; (e) a memory; and (f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

In an interrelated aspect the present disclosure is directed to a microscopy system configured to detect the rate of emergence of a biological interaction between a compound and a target fluorescent protein in a live cell comprising: (a) a stage for supporting a sample, wherein the sample comprises a population of live cells, and where the live cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a subset of the target fluorescent proteins in the sample are disposed in a detected field of view of the sample plane, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; (e) a detector device for monitoring the light-based response from the target fluorescent proteins in the presence of the compound at a plurality of time points; (e) a memory; and (f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

In certain instances of the above-described aspects, the change in movement detected is an increase in immobile trajectories indicating an increase in bound (f_{bound}) target fluorescent protein. In certain instances of the above-described aspects, wherein the movement detected is a change in: (a) the median of the jump length distribution; (b) 3^{rd} quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; and/or (j) state occupation via inference. In certain instances of the above-described aspects, the target fluorescent protein interacts in a larger molecular assembly. In certain instances of the above-described aspects, the target fluorescent protein is a ligand. In certain instances of the above-described aspects, the target fluorescent protein is a receptor. In certain instances of the above-described aspects, the biological interaction is a direct interaction. In certain instances of the above-described aspects, the direct interaction comprises binding of the compound to the target fluorescent protein. In certain instances of the above-described aspects, the biological interaction is an indirect interaction. In certain instances of the above-described aspects, the indirect interaction comprises the compound agonizing or antagonizing a larger molecular assembly comprising the target fluorescent protein.

### BRIEF DESCRIPTION OF THE DRAWINGS

The patent or application file includes at least one drawing executed in color. Copies of this patent or patent application publication with color drawing(s) will be provided by the Office upon request and payment of the necessary fee.

FIG. 1 depicts a schematic of the htSMT workflow.

FIG. 2 depicts a schematic of an exemplary image acquisition system of the present disclosure.

FIG. 3 depicts a side cross-section view of an exemplary illumination scheme, specifically highly inclined and laminated optical sheet microscopy (HILO), which can find use in connection with certain aspects of the present disclosure.

FIG. 4 depicts side cross-section views (FIG. 4, top images) of exemplary illumination schemes including HILO, highly inclined swept tile (HIST) microscopy, and single-objective lens-inclined light sheet (SOLEIL) microscopy, which can find use in connection with certain aspects of the present disclosure. FIG. 4 bottom images schematically depict the camera views of each corresponding illumination scheme.

FIG. 5 depicts a schematic of an exemplary sample handling system of the present disclosure.

FIG. 6 illustrates an exemplary system for high-throughput single-molecule imaging platform that measures protein movement in living cells.

FIG. 7 illustrates data flow through an exemplary system for a high-throughput single-molecule imaging platform that measures protein movement in living cells.

FIG. 8 depicts a plurality of images illustrating differences between mask categories and instance or semantic masks.

FIG. 9 illustrates an example computer-implemented environment in connection with the subject matter described herein.

FIG. 10 is a diagram illustrating a sample computing device architecture for implementing various aspects described herein.

FIGS. 11A-11E depicts aspects of a high throughput single molecule tracking platform. FIG. 11A depicts diffusion state probability distributions from three cell lines expressing Histone H2B-Halo, Halo-CaaX, or Halo alone, where shaded bins represent the diffusive states characteristic of each cell line. FIG. 11B depicts an example field-of-view, including mixed H2B-Halo, Halo-CaaX, and free Halo cell line single molecule images (top) and reference Hoechst image (bottom), as well as insets showing zoom-ins to individual cells and to sequential frames of individual molecules, where image intensities are equivalently scaled across panels. FIG. 11C depicts single-cell diffusive profiles extracted from FIG. 11B, colored based on similarity to the H2B-, CaaX-, or free-Halo dynamics reported in FIG. 11A. FIG. 11D depicts a heatmap representation of 103,757 cell nuclei measured from a mixture of Halo-H2B, Halo-CaaX, or Free Halo mixed within each well over 1,540 unique wells in five 384-well plates, where each horizontal line represents a nucleus and cells were clustered using k-means clustering and labels assigned based on the diffusive profiles determined in FIG. 11A. FIG. 11E depicts an ensemble state array of all tracks recovered from a mixture of Halo-H2B, Halo-CaaX, free Halo cells.

FIGS. 12A-12D provide a comparison of dynamics in a panel of steroid hormone receptors (SHR). FIG. 12A depicts an illustration of SHR function, where, under basal conditions, SHRs are sequestered in complex with HSP90 and other cofactors and upon ligand binding, the receptor dissociates from the inactive complex, dimerizes, and binds to DNA. FIG. 12B depicts a distribution of diffusive states for Halo-AR, Halo-ER, Halo-GR, and Halo-PR in U2OS cells before and after stimulation with an activating ligand, where the area in the shaded region is f_{bound}, and where the shaded error bands represent S.D. FIG. 12C depicts the selectivity of individual SHRs to their cognate ligand compared with other steroids, as determined by f_{bound}, where error bars represent SEM. FIG. 12D depicts the selectivity of individual SHRs to their cognate ligand compared with other steroids, as determined by D_{free}, where error bars represent SEM.

FIG. 13 illustrates that a screen of bioactive compounds against estrogen receptor (ER) shows reproducibility and robustness. The repeatability of screening results was assessed over two biological replicates. The plot shows the change in f_{bound} for ER from 5067 compounds. Compounds in magenta were identified as active if their magnitude of change in f_{bound} when averaged across both replicates was greater than ±0.05. Of the expected 30 positive control compounds, 26 significantly increased f_{bound} in both replicates (gray outline). Three compounds increased f_{bound} but were only present in one replicate after filtering. Exemplified positive controls including the agonist estradiol (1) and the antagonists fulvestrant (5), 4-OHT (6) and bazedoxifene (7) are exemplified. A linear regression was fit to the magenta compounds to determine slope and correlation.

FIGS. 14A-14G illustrate that selective ER modulators and degraders induce DNA binding measurable through SMT. FIG. 14A depicts diffusion state probability distribution for ER treated with 100 nM of exemplified selective ER modulators (SERMs) and selective ER degraders (SERDs), where shaded regions represent the S.D. FIG. 14B depicts the change in f_{bound} as a function of a 12-pt dose titration of fulvestrant (5), 4-OHT (6), GDC-0810 (8), AZD9496 (9), or GDC-0927 (10) with fitted curve and compounds colored as in (FIG. 14A), where error bars represent the SEM of three replicate. FIG. 14C depicts the change in f_{bound} as a function of time after agonist or antagonist addition, fitted with a single exponential and compounds colored as in FIG. 14A, where Estradiol (1, green) and DMSO added for comparison and error bars represent SEM. FIG. 14D depicts the maximum effect of SERMs and SERDs on f_{bound}, where each box represents quartiles while whiskers denote the 5-95^{th} percentiles of single well measurements, measured over a minimum of four days with at least 8 wells per compound per day. FIG. 14E depicts Fluorescence Recovery After Photobleaching (FRAP) of ER-Halo cells, treated either with DMSO alone or with 100 nM SERM/D, where curves are the mean ±SEM for 18-24 cells, colored as in FIG. 14A and error bands represent SEM. FIG. 14F depicts quantification of FRAP recovery curves to measure recovery 2 minutes after photobleaching, where whiskers denote the 5-95^{th} percentiles of single cell measurements. FIG. 14G depicts track length survival curve of ER-Halo cells treated either with DMSO alone or with SERM/D, where track survival is plotted as the 1-CDF of the track length distribution and faster decay means shorter binding times, where the inset depicts quantification of the curves in FIG. 14G, each point representing the fraction of tracks greater than 10 seconds for a single biological replicate consisting of 3-10 wells per condition and the dashed line represents the median fraction of tracks lasting longer than 10 sec for Histone H2B-Halo, which represents the upper limit of measurement sensitivity.

FIGS. 15A-15D illustrate that htSMT can be used to determine chemical structure-activity relationships. FIG. 15A depicts an example of GDC-0927 induced degradation of ER in different cell lines measured by immunofluorescence against ER, where cells were exposed to compound for 24 hours prior to fixation and image pixel intensities are equivalently scaled. FIG. 15B depicts the correlation of potency measured by ER degradation and cell proliferation in MCF7 cells (black) and T47d cells (magenta) for compounds in the GDC-0927 structural series. FIG. 15C depicts the change in f_{bound} across a 12-pt dose titration of compounds 11 through 16, colored by structure. Points are the mean ±SEM across three biological replicates. FIG. 15D depicts the correlation of potency measured by change in f_{bound} and cell proliferation in MCF7 cells (black) and T47d cells (magenta) for compounds in the GDC-0927 structural series.

FIGS. 16A-16F illustrate bioactive molecules targeting pathways associated with ER affecting ER dynamics. FIG. 16A depicts bioactive screen results with select inhibitors grouped and uniquely colored by pathway. FIG. 16B depicts the change in f_{bound} across a 12-pt dose titration of three representative compounds targeting each of HSP90, mTOR, CDK9 and the proteasome, where individual molecules are denoted by specific shapes, and error bars represent SEM. FIG. 16C depicts the change in f_{bound} as a function of time after compound addition, where estradiol treatment (black) is compared to ganetespib (blue circles) and HSP990 (blue squares), points are bins of 4 minutes, and error bars represent SEM. FIG. 16D depicts the change in f_{bound} as a function of time after compound addition, where estradiol treatment (black) is compared to HSP90 inhibitors ganetespib (blue circles) and HSP990 (blue squares); proteasome inhibitors bortezomib (red circles) and carfilzomib (red squares), points are bins of 7.5 minutes of htSMT data, marking the mean ±SEM, and the shaded region denotes the window of time used during htSMT screening. FIG. 16E depicts the track length survival curve of ER-Halo cells treated either with DMSO alone, with estradiol stimulation, or with 100 nM HSP90 or proteasome inhibition, where track survival is plotted as the 1-CDF of the track length distribution; faster decay means shorter binding times, and where the Inset Quantification of the number of tracks greater than length 2 as a function of treatment condition, where all conditions were normalized to the median number of tracks in DMSO. FIG. 16F provides a diagram summarizing pathway interactions based on htSMT results for ER, AR, and PR.

FIGS. 17A-17B provide a characterization of htSMT system performance. FIG. 17A depicts the distribution of localization error measured across multiple independent wells using Histone H2B-Halo cells, with a median localization error of 39 nm. FIG. 17B depicts the number of measurable nuclei in a 94 by 94 μm FOV, where each box plot is the distribution over wells in one 384-well plate.

FIGS. 18A-18E provide that a screen of bioactive molecules produces robust data with good assay performance. FIG. 18A depicts a boxplot showing the number of cell nuclei measured for each compound tested, where whiskers are the 1^{st} and 99^{th} percentiles. FIG. 18B depicts Z′-factor analysis for the bioactive screen, where each point represents the Z′-factor of a single 384-well plate, measuring the difference between DMSO and 25 nM estradiol treatment and plates with very low Z′-factors were removed from further analysis. FIG. 18C depicts a dose titration of estradiol for each plate in the bioactive molecules screen fit with a logistic regression to determine potency. FIG. 18D depicts the EC₅₀ values extracted from each curve fit in FIG. 18C, where the shaded region is a three-fold range in potency. FIG. 18E depicts the distribution of change in f_{bound} for control DMSO wells.

FIGS. 19A-19E illustrate that SERMs and SERDs decrease free diffusion and increase f_{bound} rapidly after addition. FIG. 19A depicts normalized occupation of diffusive states (diffusion coefficient 0.2-100 μm²/sec) for 100 nM SERD or SERM-treated samples compared with DMSO, where histograms are normalized to integrate to 1, shaded regions represent bin-wise standard deviations, and curves are the average of 3-4 biological replicates, with 8 well replicates per condition. FIG. 19B depicts results from a single-exponential association fit to the data in FIG. 15C. FIG. 19C depicts the change in diffusive state distribution as a function of time after compound addition for estadiol and fulvestrant, where each curve represents the mean of three biological replicates, and shaded regions are the bin-wise standard deviation. FIG. 19D depicts the change in f_{bound} of selective AR or GR antagonists from the bioactive screening set. FIG. 19E provides a table of results of the slow decay rate constant (k_{slow}) from SMT curve fits, where fits were performed on the aggregate of three biological replicates, and where taken together with the f_{bound} determined in 15D, an upper limit of the inferred kₒₙ can be calculated from the equation assuming all bound molecules will have k*_{off}=k_{slow}, and cells marked with an asterisk are ones in which k_{slow} could not reliably be distinguished from photobleaching.

FIGS. 20A-20D provide GDC-0927 structural variants characterized by ER degradation or cell proliferation assays. FIG. 20A depicts a western blot of the ER-expressing breast cancer cell lines MCF7 and T47d compared to ER expression in ER-null lines SK-BR-3 or U2OS, where samples were treated with fulvestrant for 24 hours prior to lysis and fulvestrant treatment leads to the degradation of ER, even when fused to HaloTag. FIG. 20B depicts exemplary compound dose titrations showing change in mean nuclear intensity as a function of compound concentration. FIG. 20C depicts an example of GDC-0927 effect on MCF7 breast cancer cell proliferation, compared with Staurosporine as a positive control. FIG. 20D depicts exemplary compound dose titrations measuring cell proliferation, normalized to DMSO-treated cells, of cells treated with analogs of GDC-0927.

FIGS. 21A-21D provides additional investigation of bioactive screening data including non-limiting exemplary cutoffs for active molecules. FIG. 21A provides examples of compound effects on ER f_{bound} from a dose titration experiment, where 92 compounds from the primary screen are ranked based on the magnitude of their effect and compounds colored in black repeatably showed dose-dependent changes; magenta compounds were inactive in a dose titration. FIG. 21B depicts an exemplary dose titration of compounds of increasing overall change in ER f_{bound}. FIG. 21C depicts two replicates of the bioactive screen as in FIG. 13, where active compounds are colored based on structural scaffold. FIG. 21D depicts a quantification of the number of compounds associated with any given cluster, where singletons (cluster 21) represent the majority of active compounds.

FIGS. 22A-22D illustrates that some pathway inhibitors modulating ER dynamics are specific to ER. FIG. 22A depicts the change in f_{bound} of ER following treatment with inhibitors of HSP90, the proteasome, mTOR, and CDK from the initial bioactive screen, where CDK inhibitors were further stratified into CDK4/6 inhibitors, CDK9 inhibitors, or inhibitors without strong selectivity for a particular family (pan-CDK), and the line represents the median for each target. FIG. 22B depicts the change in f_{bound} of ER for 97 bioactive molecules, colored by their pathway annotation, where compounds are plotted in their rank order based on their effect in ER, and error bars are the SEM of three biological replicates. FIG. 22C depicts the change in f_{bound} of AR for the same molecules from FIG. 22B, where compounds are plotted in their rank order based on their effect in ER, and error bars are the SEM of three biological replicates. FIG. 22D depicts the change in f_{bound} of PR for the same molecules from FIG. 22B, where compounds are plotted in their rank order based on their effect in ER, and error bars are the SEM of three biological replicates.

FIG. 23 depicts dose titration plots of ER(S104A/S106A/S118A) with mTOR and CDK9 compounds. Each point represents the mean and SEM of three biological replicates.

FIG. 24 depicts the f_{bound} measured for AR in the presence of an agonist at 25 nM, a potent antagonist at 10 μM, or the combination of agonist and antagonist at 25 nM and 10 μM, respectively.

FIG. 25 depicts the change in Target A movement in response to dose titrations of known Target A antagonists, where each series of shapes represents a different compound and error bars represent SEM.

FIG. 26 illustrates two representative examples of receptor tyrosine kinases, (Target B and Target C), whose movements change in response to dose titrations of known ATP-competitive or allosteric inhibitors. Each series of shapes represents a different compound normalized to DMSO and error bars represent SEM.

FIG. 27 depicts the change in Q3 Jump Length as a function of time after compound addition for additional representative targets. Proteins treated with on-target inhibitors increase or decrease protein movement, in the case of Target B or Target A respectively, within minutes post compound addition. A helicase treated with either a pathway antagonist or off-target modulator, such as the case with DNA damage induction, elicits a change in protein movement that takes several hours or more to reach maximal effect.

FIG. 28 depicts the cumulative number of trajectories as a function of imaging time for each individual cell type labeled with 10 pM JF₅₄₉. Shaded error bars represent 1 standard deviation.

FIG. 29 depicts the mRNA transcript levels in log(FPKM) of AR, ESR1, PR and NR3C1 for the engineered U2OS cell lines compared to the parental U2OS line as well as three reference breast cancer cell lines.

FIG. 30 depicts the reference gene sets induced after 24 hours of stimulation with 25 nM estradiol. The top five induced gene sets for Halo-ER ectopic expression increases were not significantly induced in the parental U2OS line.

FIG. 31 depicts a bar plot showing the −log(q value) from the top 50 most significantly induced gene sets after estradiol stimulation. Gene sets characteristic of ESR1 or the estrogen response are noted in dark gray.

FIG. 32 depicts the cumulative number of trajectories as a function of imaging time for DMSO-treated cells labeled with 20 pM JF₅₄₉. Shaded error bars represent 1 standard deviation.

FIG. 33 depicts the change in f_{bound} for 30 known ER-interaction molecules circled in FIG. 13 ordered by the magnitude of effect. Error bars are the SEM.

FIGS. 34A-34D depict that antagonists of ER (FIG. 34A), PR (FIG. 34B), AR (FIG. 34C), and GR (FIG. 34D) show distinct effects on target protein dynamics. Change in f_{bound} after addition of 1 μM antagonist either in the absence or presence of the cognate agonist. Dashed line shows f_{bound} for the vehicle-treated control.

### DETAILED DESCRIPTION

The presently disclosed subject matter relates to the development of the first industrial-scale high-throughput SMT (htSMT) techniques, systems incorporating such htSMT techniques, hardware and software related to such htSMT techniques, as well as methods of using such htSMT techniques. For example, the htSMT techniques described herein are capable of measuring protein movement in >1,000,000 cells per day. In addition, using Estrogen Receptor (ER) as a proof-of-concept system, the htSMT techniques described herein exhibit specific, robust, and reproducible results. The htSMT techniques described herein can be used for a variety of applications including, but not limited to, drug discovery activities, such as compound library screening and the elucidation of structure-activity relationships (SAR). Importantly, the htSMT techniques described herein can be used to characterize both known and novel pathway contributions to larger molecular assemblies comprising the target, such as protein signaling interaction networks.

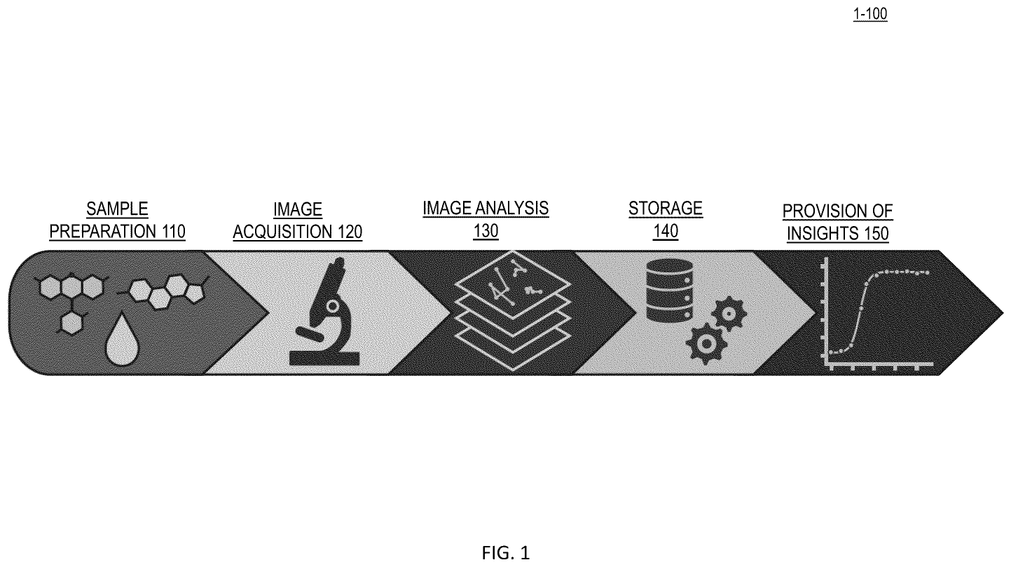

With reference to FIG. 1, aspects of the current subject matter can be implemented using an htSMT workflow. This workflow can include various phases, as will be described in further detail below, such as (i) sample preparation including reagent handling, (ii) image acquisition using imaging of the samples to generate a series of images and/or videos, (iii) image analysis through processing of these images and video using, for example, various analytics, single-emitter detection and sub-pixel localization (i.e., “super resolution imaging”), tracking, computer vision, and machine learning algorithms, (iv) storage of information (i.e., features, raw images, modified images, etc.) extracted from or otherwise characterizing or comprising the images and video, and (v) provision of insights using the stored information including biological interpretation (which can additionally or alternatively be provided using various analytics, tracking, computer vision, and machine learning algorithms).

The subject matter of the present disclosure is described with reference to the figures, where reference numbers are used to designate similar or equivalent elements throughout. The figures are not drawn to scale and they are provided merely to illustrate aspects disclosed herein. Several disclosed aspects are described below with reference to exemplary hardware, software, and applications for illustration. It should be understood that numerous specific details, relationships and methods are set forth to provide a more complete understanding of the subject matter disclosed herein. For purposes of clarity of disclosure and not by way of limitation, the detailed description is divided into the following subsections:
1. Definitions
2. htSMT Hardware
3. htSMT Software
4. Specific htSMT Applications
5. Exemplary Embodiments
6. Examples

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of”, and “consisting essentially of,” the embodiments or elements presented herein, whether explicitly set forth or not.

For the recitation of numeric ranges herein, each intervening number within the range is explicitly contemplated with the same degree of precision. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein the term “trajectory” refers to the set of spatial coordinates corresponding to the position of an observation of fluorescent protein, linked in time. In certain instances, a plurality of trajectories may be constructed algorithmically by linking a plurality of fluorescent proteins whose positions have been determined in successive time points. In certain instances, a plurality of trajectories may be constructed conservatively by linking only spots within a fixed search radius when no other links are plausible. In certain instances, a plurality of trajectories may be constructed probabilistically.

As defined herein, protein movement refers to the change in position of a plurality of fluorescent proteins. In certain instances, protein movement may be quantified by analysis of changes in spatial coordinates in sequential timepoints. Movement characterized in this way may include, but not be limited to, measurements of the jump length distribution: Given a set of protein displacements between one timepoint and a subsequent timepoint, a histogram can be constructed of the probability of each of the displacement lengths (“jump lengths”). Quantiles of this distribution can be used to describe the motion of the protein. In certain instances the quantile used is the median of the jump length distribution. In certain instances, the quantile used is the 3^{rd} quartile of the jump length distribution. In certain instances, protein movement may be quantified by analysis of trajectories. Movement characterized in this way may include, but not be limited to, measurements of the mean squared displacement as defined by the average of the square of all displacements in a trajectory, averaged over the plurality of trajectories. Movement characterized in this way may also include, but not be limited to, measurements of the trajectory length or distribution of trajectory lengths. Movement characterized in this way may also include, but not be limited to, measurements of the mean radius of gyration, as defined by the root mean square distance of all coordinates in a trajectory from the center of mass of the set of points contained in the trajectory, averaged over the plurality of trajectories. Movement characterized in this way may also include, but not be limited to, measurements of the mean bond angle, defined by the angle formed from three sequential spatial coordinates averaged over the plurality of trajectories. Movement characterized in this way may also include, but not be limited to, measurements of the diffusion coefficient maximum likelihood estimator, defined as an estimate of the maximum likelihood diffusion coefficient for the plurality of trajectories under a single-state diffusion model with constant localization error. In certain instances, protein movement may be measured by measured through analysis of the product of the link-generating algorithm. Movement characterized in this way may include, but not be limited to, the mean posterior diffusion coefficient, the mean of the posterior probability distribution of coefficients from a probabilistic linking algorithm. Movement characterized in this way may include, but not be limited to, the geometric mean posterior diffusion coefficient, the mean of the log-scaled posterior probability distribution of coefficients from a probabilistic linking algorithm. In certain instances, protein movement may be measured by measured through model-dependent analysis of the plurality of trajectories. Movement characterized in this way may include, but not be limited to, the fraction of immobile molecules (“f_{bound}”) as defined by two-state model fitting.

As used herein, the term “movement” encompasses changes in the direction as well as changes, both increases and decreases, in the speed at which a target is traveling. Accordingly, tracking movement can, in certain instances, include determining that the target is not moving, e.g., when the target either is or is essentially in a static bound state. Movement can be characterized in a variety of ways, including, but not limited to, quantifying: (a) the median of the jump length distribution (where the jump length corresponds to the observed distance the target fluorescent protein travels in consecutive frames); (b) 3rd quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; (i) trajectory length; and/or (j) state occupation via inference.

As used herein, the movement being detected, including, but not limited to, any change in movement, can occur in response to any environmental or other factor. For example, but not by way of limitation, the movement, or lack thereof, can be elicited by: (A) compound addition; (B) a change in temperature; (C) a change in oxygen concentration, e.g., introduction of a hypoxic condition; (D) mechanical stress; (E) a change in pH; and/or (F) a change in light exposure (e.g., increasing or decreasing intensity).

As used herein, the term “fluorescent protein” refers to any protein that emits a fluorescent signal. In certain instances, the fluorescent emission occurs in response to exposure to light of a particular wavelength. An example of a naturally occurring fluorescent protein is Green fluorescent protein (GFP). In certain instances, however, a protein of interest can be adapted to emit a fluorescent signal via the introduction of an encoded fluorescent tag, i.e., a protein sequence is fused to a protein of interest to render it fluorescent. In certain instances, a protein of interest can be adapted to emit a fluorescent signal through binding of a fluorescent ligand. Nonlimiting examples of such encoded fluorescent tags include: Halo tags, SNAP tags, CLIP tags, TMP tags, and SunTags. Additionally, or alternatively, a protein of interest can be adapted to emit a fluorescent signal via coupling the protein to a fluorescent dye molecule, e.g., amine- or sulfhydryl-reactive dyes.

As used herein, the term “compound” refers to any chemically-defined entity. In certain instances, the compound can be a molecule less than 1000 Da, i.e., a “small molecule”. In certain instances, the compound can be a macromolecule such as a nucleic acid. In certain instances, the nucleic acid can have a defined sequence. In certain instances the nucleic acid comprises; (A) ribonucleic acid (RNA), including, for example, modified RNA; (B) deoxyribonucleic acid (DNA), including, for example, modified DNA; as well as (C) combinations of (A) and (B). In certain instances, the nucleic acid will be a single-stranded or double-stranded small interfering nucleic acid (e.g., a double-stranded siRNA), an antisense oligonucleotide, a ribozyme, a microRNA, or an aptamer. In certain instances, the compound can be a protein. For example, but not by way of limitation, the protein compounds of the present disclosure encompass signaling proteins, e.g., protein hormones, cytokines, kinases, phosphatases, and other enzymes and transcription factors, as well as antibodies, contractile proteins, structural proteins, storage proteins, and transport proteins. In certain instances, a compound can refer to a mixture of molecules, e.g., a mixture of defined composition.

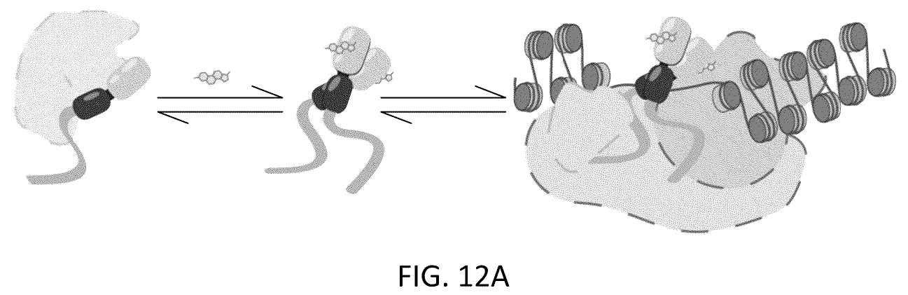

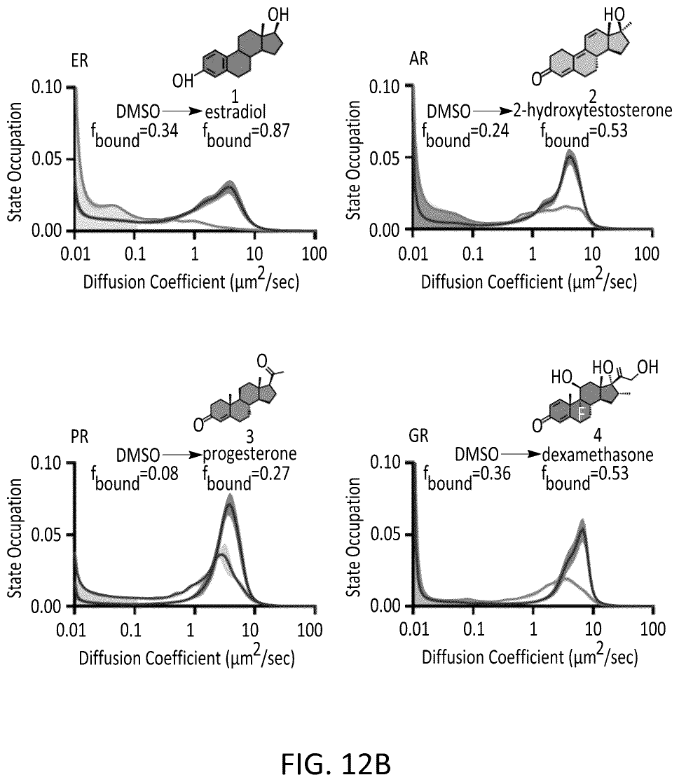

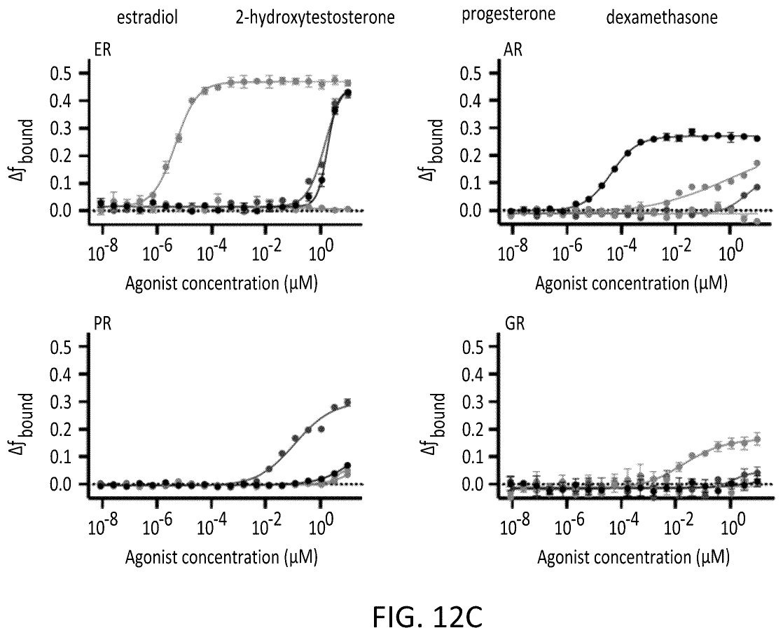

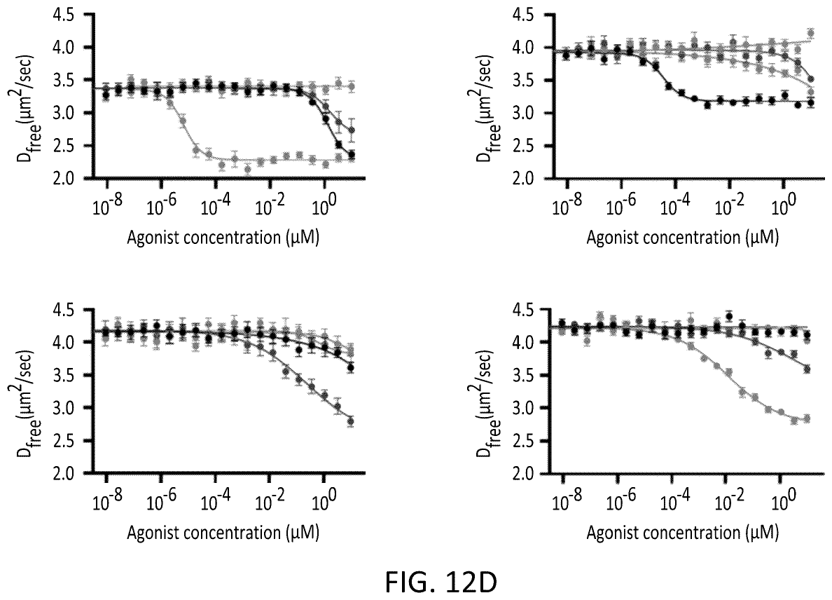

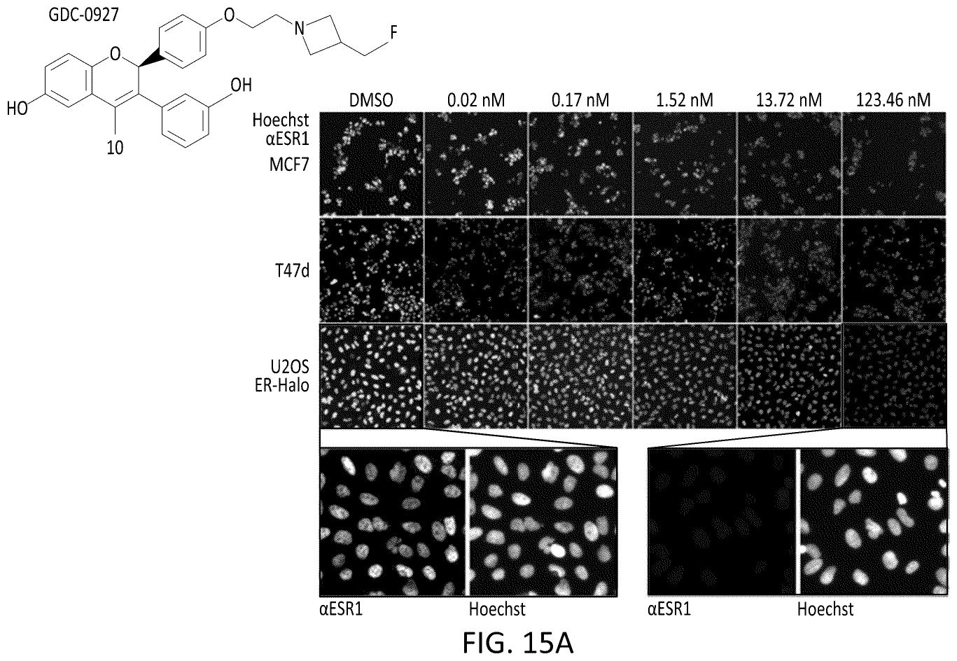

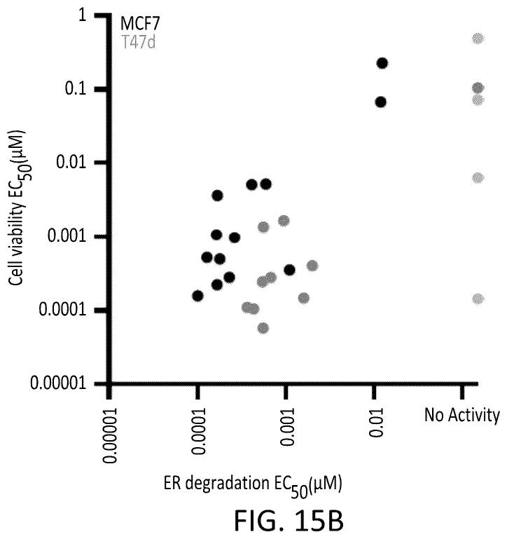

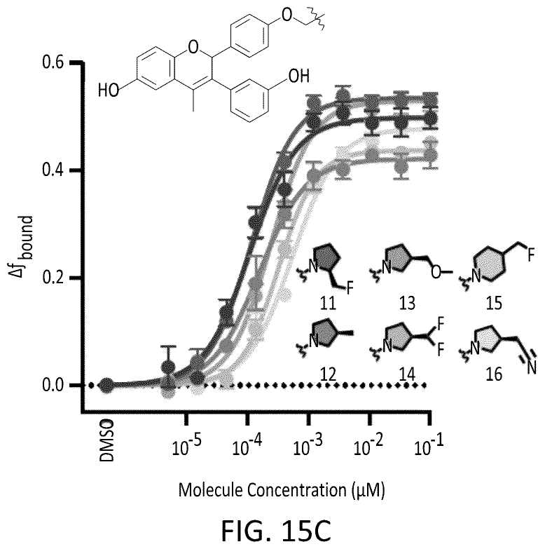

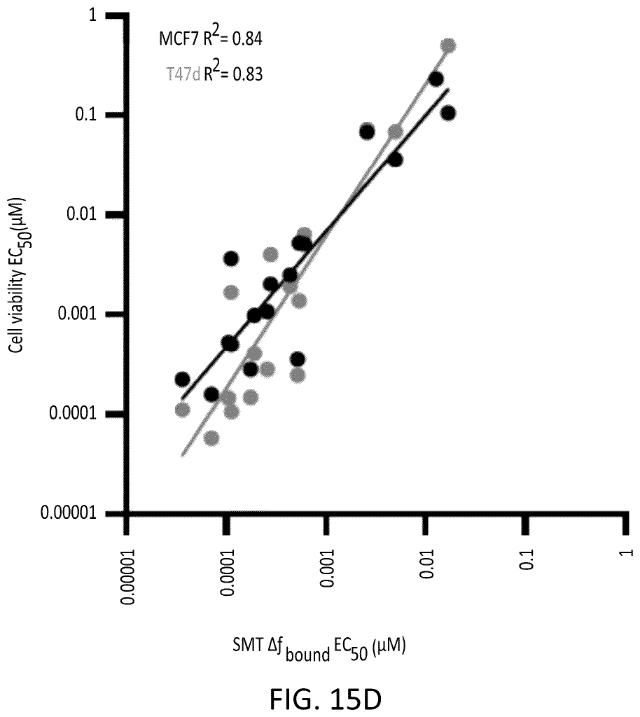

Throughout the figures and specification, certain numbers are associated with certain compounds, e.g., see FIGS. 12B, 13, 14A-14C, 15C, and Example 1. Specifically, the compounds and their respective numbers are: estradiol (1); 2-hydroxytestosterone (2); progesterone (3); dexamethasone (4); fulvestrant (5); 4-OHT (6); bazedoxifene (7); GDC-0810 (8); AZD9496 (9); and GDC-0927 (10). In addition, FIG. 15C includes a GDC-0927 structural series wherein the specific modifications to GDC-0927 are illustrated and numbered from (11)-(16).

### 2. htSMT HARDWARE

### 2.1. Image Acquisition Systems

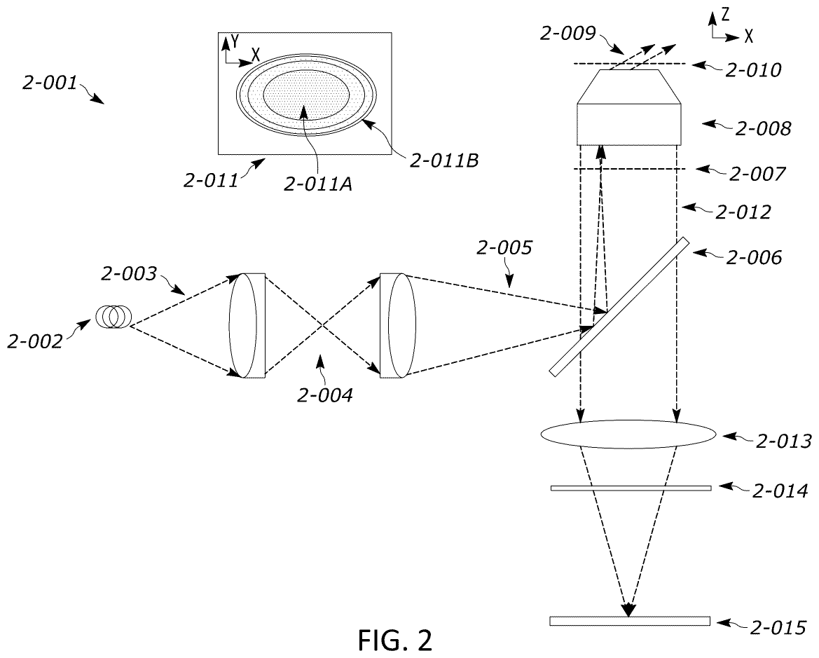

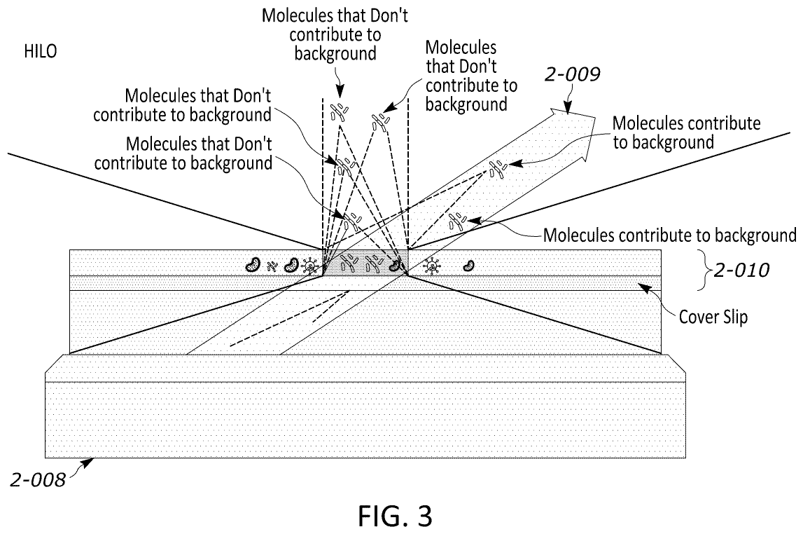

With reference to FIG. 1, aspects of the current subject matter can be implemented using an htSMT workflow, where such workflow incorporates systems for image acquisition using imaging of samples to generate a series of images and/or videos. For example, but not by way of limitation, FIG. 2 depicts a schematic of an exemplary image acquisition system of the present disclosure. The exemplary image acquisition system (2-001) comprises: a light source and single mode fiber (SMF) (2-002) configured to emit light (2-003), which is relayed by one or more optical elements in an optical relay (2-004), the optical relay being configured to shape the light emitted from the light source to form a shaped beam (2-005); and one or more optical elements (2-006), e.g., a dichroic mirror, configured to direct the shaped beam to an objective (2-008), whereby the sample plane (2-010) is illuminated by an inclined beam (2-009), resulting in the emission of light from the sample (2-012), e.g., fluorescence emission, which is focused by the objective (2-008) and one or more optical elements (2-013), e.g., a tube lens, and passed through an emission filter wheel (2-014) to an image collection system (2-015), e.g., a detector device.

### 2.1.1. Light Source

With reference to the exemplary image acquisition system of FIG. 2, the system comprises a light source (2-002) configured to emit light (2-003). The light source (2-002), in certain implementations of the image acquisition systems disclosed herein, can be configured to emit light of a single wavelength. In certain implementations of the image acquisition systems disclosed herein, the light source (2-002) can be configured to emit light of two, three, four, five, or more individual wavelengths. In certain implementations, the wavelength(s) of light emitted by the light source are predetermined. For example, but not by way of limitation, the wavelength(s) can be predetermined such that the emitted light elicits fluorescence emission when illuminating a sample, e.g., a sample comprising a fluorescent protein. In certain instances, the wavelength(s) employed in connection with the methods described herein will fall within a range of 400 nm to 650 nm. In certain instances, the light source (2-002) will emit light having a wavelength between 400 nm to 408, between 550 nm to 565 nm, or between 638 nm to 650 nm. In certain non-limiting implementations, the light source (2-002) is configured to comprise three lasers with nominal central wavelengths 405 nm, 560 nm, 640 nm that could vary within absorption band of the fluorophores used. In certain instances, the 405 nm wavelength is used to excite Hoechst dye. In certain instances, a 560 nm wavelength is used to excite dyes (e.g., JF549) attached to HaloTag.

In certain non-limiting implementations, the light source (2-002) is used to catalyze photochemical reactions. For example, but not by way of limitation, the wavelength(s) and illumination intensities can be such that cleavage of a chemical bond occurs. As an additional example, but not by way of limitation, the wavelength(s) and illumination intensities may induce the adoption of a non-radiative dark state (i.e., “photobleached molecule”). As an additional example, but not by way of limitation, the wavelength(s) and illumination intensities may induce radiative or non-radiative energy transfer between fluorophores within the sample.

In certain implementations of the image acquisition systems described herein, the light source (2-002) can be configured to deliver a predetermined amount of power to the back focal plane of the objective (2-007). For example, but not by way of limitation, the light source (2-002) delivers greater than 10 mW with respect to certain wavelengths, e.g., 405 nm, and/or greater than 150 mW with respect to other wavelengths, e.g., 640 nm. Additionally, or alternatively, in instances where the light source (2-002) comprises three lasers emitting at 405 nm, 560 nm, and 640 nm wavelengths, respectively the light source (2-002) can be configured to deliver predetermined amounts of power, to the back focal plane of the objective (2-007). For example, but not by way of limitation the 405 nm can be configured to deliver <10 mW; the 560 nm can be configured to deliver >150 mW; and the 640 nm can be configured to deliver >50 mW).

In certain implementations of the image acquisition systems described herein, the light source (2-002) is configured to emit pulsed light. For example, but not by way of limitation, the light source (2-002) can be configured to emit stroboscopic pulsed light. In certain, non-limiting implementations, the light source (2-002) will emit 2 msec stroboscopic pulsed light. Additionally, or alternatively, the light can be pulsed in synchrony with the start of frame acquisition, as described in detail below.

The emission of light (2-003) by the light source (2-002) and the direction of that light to the optical relay (2-004), can, in certain implementations of the image acquisition systems disclosed herein, be facilitated using a single mode fiber. Additionally, or alternatively, a multimode fiber with a predetermined core shape for sample illumination can be used.

In certain implementations of the image acquisition systems described herein, for example with respect to systems configured for high throughput sample analysis, the light source (2-002) can be configured to exhibit low drift in power output. In certain implementations, such low drift configurations increase sample processing consistency to facilitate high throughout analyses. For example, but not by way of limitation, such low drift power output configurations maintain power output within about 0% to about 15% variation, about 0% to about 10% variation, about 10% variation, about 9% variation, about 8% variation, about 7% variation, about 6% variation, about 5% variation, about 4% variation, about 3% variation, about 2% variation or about 1% variation.

In certain instances, such low drift power output configurations that maintain power output within about 0% to about 15% variation, about 0% to about 10% variation, about 10% variation, about 9% variation, about 8% variation, about 7% variation, about 6% variation, about 5% variation, about 4% variation, about 3% variation, about 2% variation or about 1% variation in the context of changing ambient (room) temperature, e.g., 17° C. +/−5° C. In certain instances, this is achieved using temperature sensors and/or close-loop heaters to maintain internal light source (e.g., laser engine) temperatures stable, thereby reducing output power drift. For example, but not by way of limitation, the light source can be thermally insulated from the fluctuations of the ambient temperature using an insulated enclosure design. Additionally, or alternatively, closed-loop heaters can be strategically placed at specific locations in the system, e.g., the fiber coupler to reduce output drift. Additionally, or alternatively, water jackets and/or chillers can be used to reduce heat build-up from the laser heads. Moreover, these thermal controls, used individually or in combination, result in shorter warm up times to reach operating steady state and maintained more stable internal operating temperatures when lasers would be powered off and on.

### 2.1.2. Optical Elements & Sample Illumination

With reference to the exemplary image acquisition system of FIG. 2, the system comprises a light source (2-002) configured to emit light (2-003), which is relayed by one or more optical elements in an optical relay (2-004), the optical relay being configured to shape the light emitted from the light source to form a shaped beam (2-005).

While FIG. 2 depicts an exemplary HILO implementation for use in the htSMT workflows described herein, the htSMT workflows described herein can incorporate a variety of illumination strategies. For example, but not by way of limitation, the htSMT workflows described herein can be implemented using HILO, Total Internal Reflection Fluorescence (TIRF), HIST, or SOLEIL microscopy illumination strategies. One of skill in the art would understand, based on the htSMT workflows described herein, advantageous ways to adapt TIRF, HIST, or SOLEIL illumination strategies for use in the instant methods. For example, the optical elements of any particular optical relay (2-004) can be selected and configured to produce the appropriately shaped beam (2-005) as well as provide for the appropriate translation of that beam, e.g., when a HIST illumination strategy is employed. Additionally, or alternatively, one of skill in the art would understand, based on the htSMT workflows describe herein, how to configure the necessary optical elements to achieve a TIRF illumination strategy. For example, optical elements can be employed to incline the beam so steeply that its critical angle is hit, thereby propagating an evanescent wave through the cover glass to illuminate the sample in close proximity to the cover glass.

In certain, non-limiting implementations of the optical relays (2-004) of the presently disclosed image acquisition systems, the optical relay (2-004) will comprise one or more lenses. For example, but not by way of limitation, the selection and orientation of lenses in the optical relay (2-004) will be configured to appropriately shape the light beam being directed to the sample. In certain non-limiting implementations, the optical relay (2-004) will comprise a lens having a predetermined focal length, e.g., 80 mm, to collimate the emitted light (2-003) from the light source (2-002). Additionally, or alternatively, the optical relay (2-004) will comprise a lens or series of lenses, e.g., a telescope system, to shape the light beam. The particular focal length(s) of the lens or series of lenses will be predetermined to produce an appropriately shaped light beam.

In implementations of the htSMT workflows described herein where the image acquisition system is configured to incorporate a HIST microscopy-based illumination system, the optical relay can be configured to include a telescope comprising two cylindrical lenses (e.g., f=400/250 mm and f=50 mm) to generate a tile beam compressed 8× or 5×, which, in certain implementations, is relayed by another telescope system (e.g., f=60 mm and f=150 mm) before being passed through an additional lens (e.g., f=400 mm). In such HIST microscopy-based illumination system implementations, the optical relay (2-004) can comprise one or more optical elements or assemblies configured to translate the light beam relative to the imaging plane of the sample to be analyzed, e.g., in a direction orthogonal to the longer dimension of the light beam. For example, but not by way of limitation such optical elements or assemblies configured to translate the light beam relative to the imaging plane of the sample to be analyzed can comprise a galvo mirror. Additionally, or alternatively, such optical elements or assemblies configured to translate the light beam relative to the imaging plane of the sample to be analyzed can comprise a computer-controlled motor.

With reference to the exemplary image acquisition system of FIG. 2, the system comprises an optical relay (2-004) configured to shape the light emitted from the light source to form a shaped beam (2-005), which is then directed by an optical element (2-006), e.g., a dichroic mirror, configured to direct the shaped beam to an objective (2-008), whereby the sample plane (2-010) is illuminated by an inclined beam (2-009). Inset is the illumination view (2-011) with respect to the X and Y axis of the sample plane, comprising a peak intensity core (2-011A) that progressively drops off with a Gaussian profile to a lower intensity outer edge (2-011B).

In certain, non-limiting implementations of the image acquisition systems of the present disclosure, an objective (2-008) directs the inclined beam (2-009) on the sample plane (2-010) to be analyzed. In certain, non-limiting implementations of the image acquisition systems of the present disclosure the objective (2-008) is a water immersion objective. The use of a water immersion objective facilitates high throughput sample analysis by eliminating the oil present in connection with the use of oil immersion objectives, thereby allowing for consistent sample handling and imaging. For example, but not by way of limitation, the objective can be a 60× 1.27 NA water immersion objective (Nikon). In certain implementations of the workflows described herein, the water immersion objective (2-008) will be heated by a heating element. For example, such heating element will maintain the water immersion objective (2-008) at a temperature sufficient to avoid inducing a change in temperature of the sample contained in the sample plate (2-021).

### 2.1.3. Image Acquisition

In certain non-limiting implementations of the image acquisition systems of the present disclosure, the objective (2-008) is also used to focus the fluorescence emitted by the sample in response to the illumination provided by the inclined beam (2-009). In certain instances, however, a second objective is employed to focus the fluorescence emitted by the sample in response to the illumination provided by the inclined beam (2-009). In certain, non-limiting implementations, the objective-focused fluorescence emission (2-012) is passed through an emission filter (2-014), e.g., a bandpass emission filter matched to the spectrum of the fluorophore under observation and mounted in high-speed filter wheel (Finger Lakes Instruments) and collected by a detector device (2-015). In certain, non-limiting implementations, the objective-focused fluorescence emission is directed to an optical relay prior to collection by the detector device (2-015). For example, but not by way of limitation, such an optical relay can comprise one or more lenses and one or more additional optical elements, e.g., an element configured to reject additional scattered light, prior to collection by the detector device (2-015). In certain, non-limiting implementations, the objective-focused fluorescence emission is directed through another diachroic mirror to split the emission over multiple regions of the detector (2-015), where the detector device can be a CMOS camera, e.g., a back illuminated CMOS camera (Prime 95b, Teledyne).

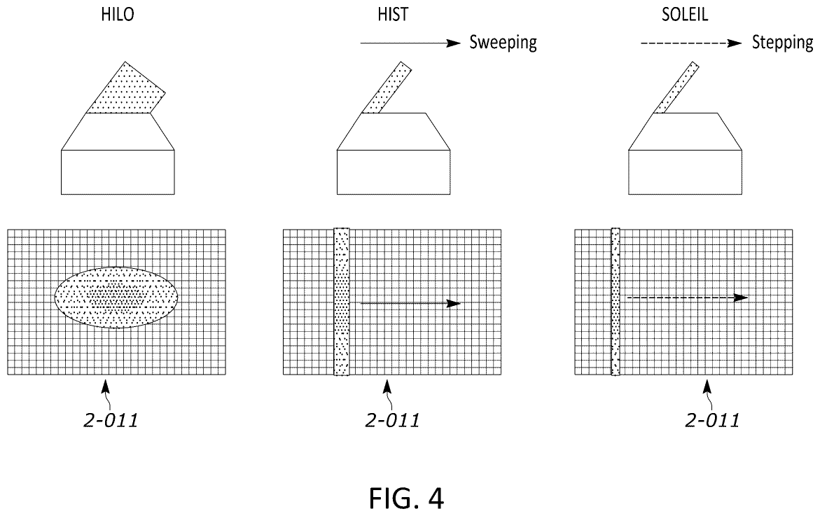

In certain, non-limiting implementations where the image acquisition system is configured to incorporate a HIST or SOLEIL microscopy-based illumination system, the detector device can be configured to synchronize detection with the translation of the inclined beam (2-009) across the sample. Such synchronization is schematically depicted in FIG. 4, lower images, associated with HIST and SOLEIL implementations where the “active pixel” corresponds to the aspect of the detector device actively collecting in synchrony with the translation of the inclined beam (2-009). For example, but not by way of limitation, the detector device can be a CMOS camera, e.g., a back illuminated CMOS camera (Hamamatsu Fusion BT).

In certain implementations of the image acquisition systems of the present disclosure, the CMOS camera can be run such that, for each field of view, a series of SMT frames is collected. For example, but not by way of limitation, 1-100,000 SMT frames, 1-50,000 SMT frames, 1-20,000 SMT frames, 1-10,000 SMT frames, 1-1,000 SMT frames, 1-500 SMT frames, 5-250 SMT frames, 10-200 SMT frames, 100-200 SMT frames, or 200 SMT frames are collected per field of view. In certain implementations, the CMOS camera can be configured to run at a frame rate of from 0.5 to 1000 Hz. In certain implementations, the CMOS camera can be configured to run at a frame rate of about 100 Hz.

In certain, non-limiting implementations of the image acquisition systems of the present disclosure, the detector device is configured to transmit a signal with each frame to trigger other components of the imaging system. For example, but not by way of limitation, the detector device may trigger the illumination from the light source (2-002) so as to collect fluorescence emission associated with stroboscopic laser pulses. For example, but not by way of limitation, such fluorescence emission collection is associated with 10 to 100 msec frames and a 2 msec stroboscopic laser pulse. In certain embodiments, fluorescence emission collection is associated with a stroboscopic laser pulse of about 1 to about 4 msec, e.g., about 1 to about 3 msec or about 2 to about 3 msec stroboscopic laser pulse, where the duration of the stroboscopic laser pulse can be selected based on the frame rate employed (e.g., 10 to 100 msec frames).

In certain embodiments, the imaging acquisition system can be configured to detect a predetermined field of view. In certain embodiments, the detected field of view can have a size of about 50 μm to less than 100 μm in a first dimension by about 50 μm to less than 100 μm in a second dimension. For example, but not by way of limitation, the detected field of view can have a size of about 94 μm in a first dimension by about 94 μm in a second dimension.

In certain implementations, the detector device can be used to collect fluorescence emission at multiple wavelengths. For example, but not by way of limitation, fluorescence emission of additional fluorophores can be collected at the same frame rate or different frame rates for the same fields of view to provide downstream registration of SMT tracks to other cellular components, e.g., nuclei. Additional channels of the detector device can be used as desired to expand the number of simultaneously captured fluorescence emissions for the same fields of view to provide downstream registration of SMT tracks to other cellular components, e.g., nuclei.

### 2.2. Sample Handling

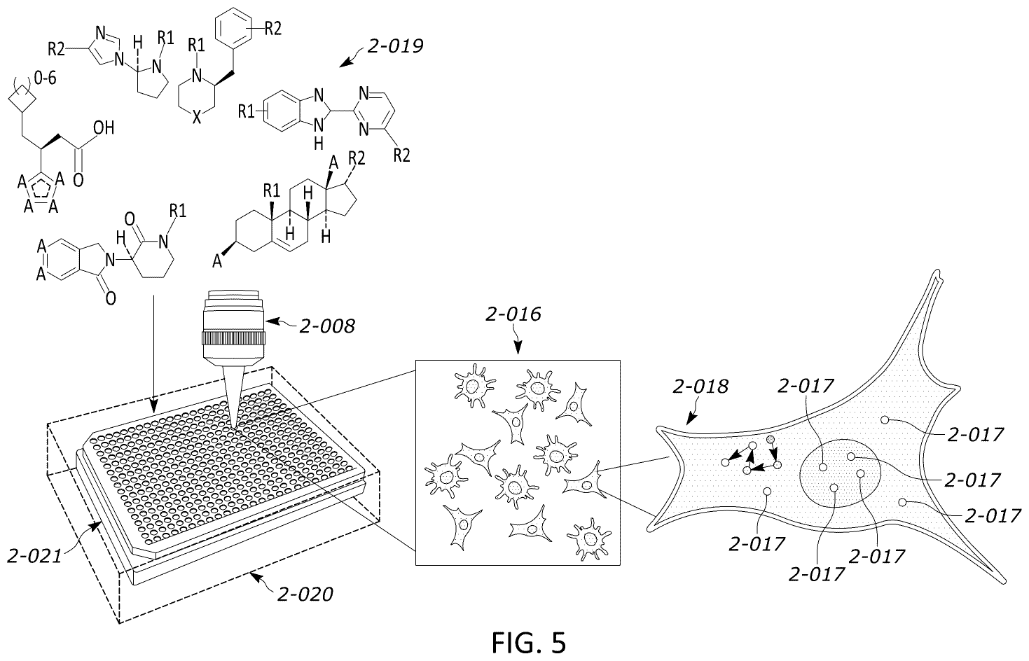

With reference to FIG. 1, aspects of the current subject matter can be implemented using an htSMT workflow, where such workflow incorporates systems for sample preparation, including reagent handling. For example, but not by way of limitation, FIG. 5 provides a schematic representation of a sample plate (2-021) comprising a plurality of wells (2-016) in which samples can be prepared and analyzed. FIG. 5 also provides a schematic representation of components of a sample, e.g., a cell (2-018) and fluorescent proteins (2-017) within the cell. As noted herein, however, FIG. 5 is not intended to convey scale, e.g., each sample present in a well (2-016) can comprise thousands of cells and each cell can comprise numerous fluorescent proteins. FIG. 5 also schematically illustrates the ability of sample handling systems of the present disclosure to add additional reagents (2-019) to sample in a sample plate (2-021). Such reagent addition can be handled by robotic manipulations, such as, but not limited to, the translation of robotic fluid handling systems relative to the individual wells (2-016) of the sample plate (2-021), the translation of the sample plate (2-021) itself, or combinations of both.

In certain implementations of the image acquisition system, the sample plate (2-021) may be maintained in a temperature-controlled environment through an environmental control area (2-020). For example, but not by way of limitation, the sample may be maintained at 22-50° C. In certain implementations of the image acquisition system, the sample plate (2-021) may be maintained in a humidity-controlled environment through an environmental control area (2-020). For example, but not by way of limitation, the sample may be maintained at 20%-95% humidity. In certain implementations of the image acquisition system, the sample plate (2-021) may be maintained in a defined gas environment through an environmental control area (2-020). For example, but not by way of limitation, the sample may be maintained at 5% CO₂.

### 2.2.1. Cell Lines & Cell Culture

With reference to FIG. 5, a particular advantage of the htSMT systems described herein is that living cells (2-018) can be assayed to facilitate the tracking of activity, mobility, and diffusive behaviors of proteins within the crowded living cellular environment. As shown in FIG. 5, the htSMT systems of the present disclosure can be used to track fluorescently labeled proteins in a sample comprising a plurality of cells. Exemplary cells (e.g., cell lines) that find use in connection with the htSMT systems described herein are considered if the sample (e.g., containing such cells) can be brought into focus by the objective (2-008) for sufficient time as to direct the fluorescence emission of fluorophores onto the detector (2-015). For example, but not by way of limitation, cells may adhere to coverglass directly. As an additional example, but not by way of limitation, cells may be induced to adhere to the coverglass after treating the coverglass with an extracellular matrix material (e.g., fibronectin, collagen, poly-D-lysine, laminin, matrigel, vitronectin, etc.). As an additional example, but not by way of limitation, cells may be induced to adhere to the coverglass after treating the coverglass with plasma.

Exemplary cells, e.g., cell lines, may be selected so as to minimize non-fluorophore emissions reaching the detector. In certain embodiments, cells for use in the present disclosure can be mammalian, bacterial or fungal cells. In certain embodiments, the cells are mammalian cells. In certain embodiments, the cells can be obtained from preserved tissue, e.g., fixed tissue, from frozen tissue e.g., frozen tissue samples, or from fresh tissue, e.g., fresh tissue samples. In certain embodiments, the cells and/or a sample containing cells can be obtained from a subject. In certain embodiments, the cells can be obtained from a malignancy of a tissue or a tumor, e.g., the cells can be present within a tumor sample (e.g., a section of a tumor). In certain embodiments, the cells can be obtained from cell lines. For example, but not by way of limitation, particular cell lines that find use in connection with the htSMT systems described herein included: U2OS cells (ATCC Cat. No. HTB-96), MCF7 cells (ATCC Cat. No. HTB-22), T47d cells (ATCC Cat. No. HTB-133) and SK-BR-3 cells (ATCC Cat. No. HTB-30). In certain embodiments, the cells can be present in a three-dimensional structure such as an organoid or a spheroid. In certain embodiments, the cells can be present in an organoid.

In certain implementations of the htSMT systems of the present disclosure, the cells to be used are cultured as necessary to provide sufficient cell numbers to achieve the desired high throughput analyses. For example, but not by way of limitation, cells, e.g., U2OS cells (ATCC Cat. No. HTB-96), MCF7 cells (ATCC Cat. No. HTB-22), T47d cells (ATCC Cat. No. HTB-133) and SK-BR-3 cells (ATCC Cat. No. HTB-30), can be grown in DMEM (Cat. No. 1056601, Gibco DMEM, high glucose, GlutaMAX Supplement, Thermofisher) supplemented with 10% Fetal Bovine Serum (Cat. No. 16000044, Thermofisher) and 1% pen-strep (Cat. No 15140122, Thermo Fisher) and maintained in a humidified 37° C. incubator at 5% CO₂ and subcultivated approximately every two to three days. Additional culture strategies that would be appropriate for the cell lines and uses outlined herein would be known those of skill in the relevant art.

In certain implementations of the htSMT systems of the present disclosure, the cells comprise one or more fluorescent protein. The selection of the specific protein(s), as well as the manner in which it fluoresces, e.g., is it to be labeled via coupling to a dye or via the inclusion of an encoded fluorescence tag, will likely differ depending on the particularities of a specific investigation. For example, but not by way of limitation, one approach for labeling proteins that finds use in connection with the htSMT systems described herein is a HaloTag fusion strategy. For example, but not by way of limitation, one approach for labeling is a fluorescent protein. For example, but not by way of limitation, on approach for labeling is a photo-convertible fluorescent protein. For example, but not by way of limitation, on approach for labeling is a photoactivatable fluorescent protein. For example, but not by way of limitation, one approach for labeling proteins is a SNAPtag fusion. For example, but not by way of limitation, one approach for labeling proteins is a CLIPtag fusion. For example, but not by way of limitation, one approach for labeling proteins is through a fluorophore ligase system. For example, but not by way of limitation, one approach for labeling proteins is via FLASH or ReAsH tetracysteine motif. For example, but not by way of limitation, one approach for labeling proteins is through strain-promoted alkyne-azide cycloaddition of a fluorophore. For example, but not by way of limitation, one approach for labeling proteins is through inducing cellular uptake of fluorescent proteins generated separately. In certain implementations of the htSMT systems of the present disclosure, the cells comprise one or more fluorescent glycoprotein. In certain embodiments, one approach for labeling proteins uses a gene-editing system, e.g., a CRISPR-based editing system. For example, and not way of limitation, a nucleic acid encoding a fluorescent protein (e.g., a fluorescent tag such as a HaloTag) can be inserted into the gene or upstream or downstream from the gene encoding the protein to be labeled to generate a protein that is fluorescently labeled with a HaloTag (e.g., at its C- or N-terminus).

While one of skill in the art can implement a HaloTag fusion-approach in a number of ways, one exemplary approach is to transfect mammalian expression vectors containing the fusion gene (i.e., a protein of interest fused in frame with a HaloTag sequence) under the control of a weak L30 promoter and containing a Neomycin resistance marker in the cell line of interest, e.g., U2OS cells. In certain implementations, such transfection can be accomplished when the cells are at 70% confluence using FuGENE 6 (Cat. No. E2691, Promega). In certain implementations, transfected cells can then be selected with the appropriate selection agent, e.g., G418 (Cat. No. 10131027, Thermo Fisher), at the appropriate concentration, e.g., at 500 μg/mL. In certain implementations, cells can then be clonally isolated. Clones expressing the desired fusion gene can be determined first by staining with 100 nM JF₅₄₉-HTL (Cat. No. GA1110, Promega) and 50 nM Hoechst 33342and identifying clones with the expected distribution of JF₅₄₉ signal. An alternative exemplary approach is to transfect cells with ribonucleoprotein (RNP) complexes included sgRNAs targeting a genomic sequence encoding the N- or C-terminal region of a target protein and Cas9 protein in combination with one or more linear dsDNA donors. In certain embodiments, each donor consists of 200-300 bp homology arms specific for each target, a codon optimized HaloTag sequence and a TEV linker (ENLYFQG) between the target and HaloTag. In certain implementations, between three and six clones can be subsequently tested using SMT conditions for response to a control compound, and the most homogenous clones can then be subsequently expanded for further testing.

While the htSMT workflows of the instant application are described generally with respect to implementations that track the impact of a compound on a target fluorescent protein, the htSMT workflows described herein are equally applicable to the tracking and analysis of fluorescent target compounds. For example, but not by way of limitation, the compounds described herein can either themselves be fluorescent or can be modified to facilitate fluorescent detection. Moreover, changes in the movement of the fluorescent compound can be utilized to determine the SMT profile of the compound itself. All analysis strategies described herein with respect to the tracking of target fluorescent proteins are therefore also applicable to results obtained by tracking the compounds themselves.

### 2.2.2. Single Molecule Tracking Sample Preparation

With reference to FIG. 5, aspects of the current subject matter can be implemented using an htSMT workflow whereby cells (2-018) are seeded on plates (2-021), e.g., tissue culture treated 384-well glass-bottom plates, although other plate types can find use in connection with the approaches outlined herein, including, but not limited to single chambers, 9-well glass-bottom plates, 24-well glass-bottom plates, 96-well glass-bottom plates, 1536-well glass-bottom plates, and 3456-well glass bottom plates, as well as plates made of alternative materials, e.g., plates made partially or entirely of plastic. In certain implementations, the cells (2-018) are seeded at 1 to 20,000 cells per well (2-016), e.g., at 50 to 10,000, at 100 to 9,000, at 250 to 8500, at 500 to 7500, at 750 to 7000, at 2500 to 6500, or at 6000 cells per well. Seeded cells can then be incubated under conditions desirable for adhesion, e.g., overnight at 37° C. and 5% CO₂. To enable fluorescence emission, cells can be incubated with a sufficient amount of label, e.g., in the case of HaloTag fusions, 0.1-100 pM of JF₅₄₉-HTL (Cat. No. GA1110, Promega) and 50 nM Hoechst 33342 (for labeling nuclei) for an hour in complete medium can provide desirable results.

In certain implementations, htSMT strategies described herein, the cells are then washed, e.g., three times in DPBS and twice in imaging media. In certain implementations, the imaging media is prepared to facilitate fluorescence emission, e.g., fluoroBrite DMEM media (Cat. No. A1896701, Thermo Fisher), and can be supplemented with GlutaMAX (Cat. No. 35050079, Thermo Fisher) and the same serum and antibiotics as growth media.

Where appropriate, compounds can be added to the samples to test their impact on a particular fluorescent protein via SMT. In certain implementations, compounds can be serially diluted in an Echo Qualified 384-Well Low Dead Volume Source Microplate (0018544, Beckman Coulter) to generate dose-titration source material. Compounds can then be administered, e.g., at a final 1:1000 dilution in cell culture medium. In certain implementations of the htSMT strategies described herein, each dose of a compound will have at least two replicates per plate as well as three plate replicates. In addition, in certain implementations of the htSMT strategies described herein, 20 DMSO control wells and two no-dye control wells can be randomized across each plate (2-012). In certain implementations, compounds can be allowed to incubate for 0 to 48 hours prior to image acquisition, e.g., one hour at 37° C.

### 3. htSMT SOFTWARE

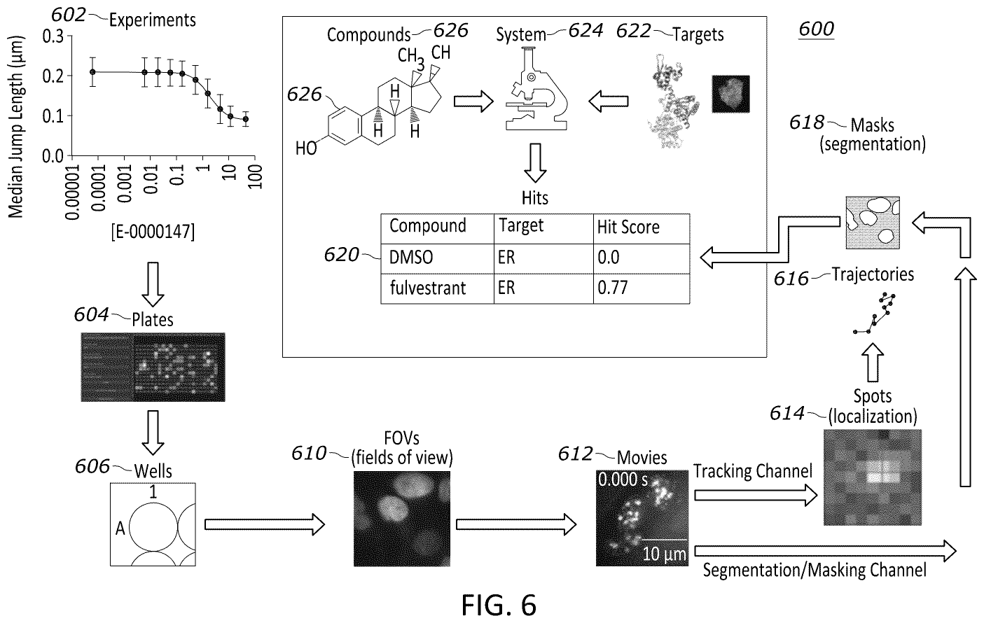

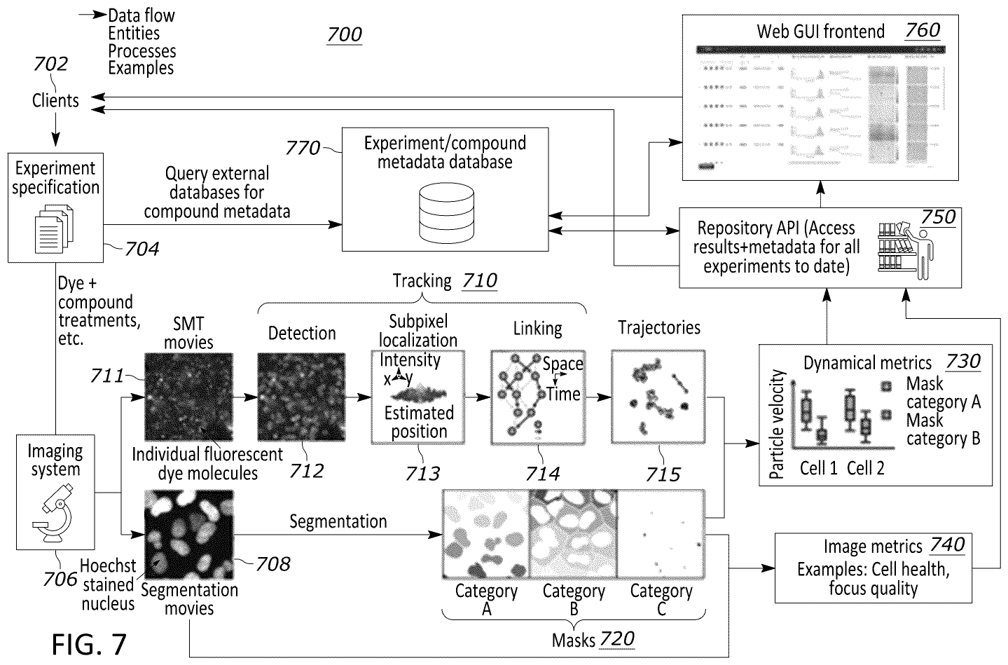

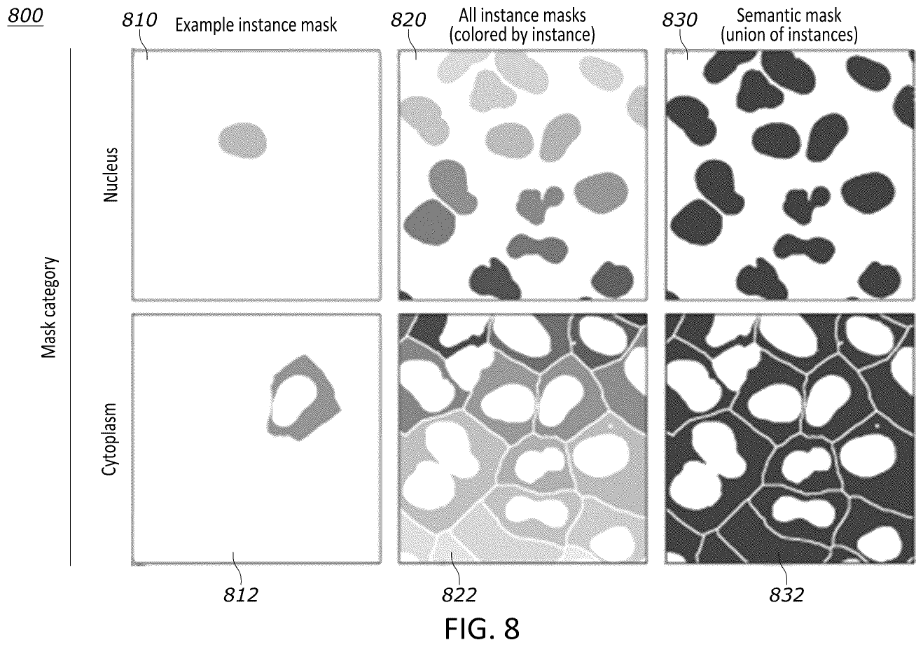

FIG. 6 illustrates an example system 600 for a high-throughput single-molecule imaging platform that measures molecule movement in living cells. Experiments 602 can be performed to collect large amounts of data from a plurality of living cells (e.g., using imaging system 624 to identify compounds 626 and/or targets 622). The experiments 602 can include the application of various identifiers to molecules of interest such as labels which can be subsequently fluoresced or otherwise detected (e.g., using a laser or other light source). The biological samples forming part of such experiments 602 can be organized into plates 604 having a plurality of wells 606. Each well 606 can have one or more associated fields of view (FOVs) 610. FOVs 610 can be locations within or corresponding to a single well 606. A sequence of images can be generated for the FOVs 610 to result in one or more movies 612, which can include SMT movies as well as non-SMT movies. SMT movies can be used to track the paths of individual labeled molecules such as proteins, generating a plurality of trajectories. Each trajectory may be comprised of a plurality of spots 614, which include the spatiotemporal coordinates of a labeled molecule at a particular time (as described in further detail in FIG. 7). Separately from the tracking, and in some instances in parallel with the tracking, the movies 612 can be utilized to identify molecules through the use of machine-learning and/or computer vision-based image segmentation to generate masks 618. Masks 618 are spatial regions within a FOV 610 produced by the segmentation. Each mask 618 can belong to a mask category, which is described in more detail in FIG. 8.

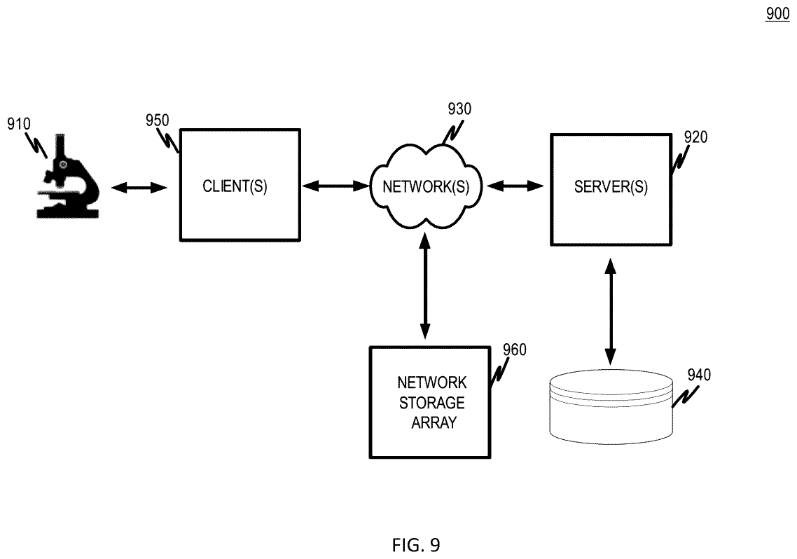

Data associated with channels (e.g., tracking channel and two segmentation/masking channel) can be combined to generate a plurality of metrics 620 associated with various aspects of the samples. In other words, the trajectories 616 (e.g., trajectory data) can be combined with the machine learning processed image segmentation data and further analyzed using statistical/machine learning methods. Processing of the combined data can be used to generate metrics 620 such as hit scores associated with compounds and/or targets within a biological sample that may be stored in a database structure, as further described in FIG. 9.

FIG. 7 illustrates data flow through an example system 700 for a high-throughput single-molecule imaging platform that measures protein movement in living cells. Experiment specifications 704 that define experiments 602 can be provided as data input via one or more clients 702. For example, each experiment 602 can be collected with accompanying stains (e.g., Hoechst or Potomac Red) that are used for downstream analysis including segmentation 618. The experiment specifications 704 can define various parameters for the experiments 602 such as stains, dyes, compounds, treatments, and the like. As previously described in FIG. 6, imaging system 706 (e.g., imaging system 624) can capture a sequence of images that generate one or SMT movies 711 and/or non-SMT movies or segmentation movies 708 (e.g., movies 612) which characterize molecular movement. The SMT movies 711 can characterize movement of individual fluorescent molecules and/or contain images of individual fluorescent molecules. The segmentation movies 708 can comprise a sequence of images that characterize movement of labeled molecules and/or component thereof. It will be appreciated that Hoechst staining is only one technique that can be used to label molecules and that different and/or multiple labeling techniques such as Potomoc Red can be utilized depending on the desired configuration. For example, MitoTracker Deep Red can be used to label mitochondria), concanavalin A-dye conjugates can be used to label endoplasmic reticulum, SYTO 14 can be used to label nucleoli, phalloidin can be used to label actin, and the like.

The SMT movies 711 can be analyzed to perform operations relating to molecule tracking 710 which can include detecting 712, subpixel localization 713, and linking 714 to identify trajectories 715 of molecules across various images within the SMT movies 711. More specifically, during detection 712 one or more spots within the SMT movies 711 can be detected or recovered. Each spot can be equipped with spatiotemporal coordinates. These spatiotemporal coordinates can be estimated by using subpixel localization techniques 713. Linking 714 can be performed on the spots to ultimately identify trajectories 715.

Links, as used herein, are potential associations between two spots. Each link is directed, beginning at one spot and ending at another. A “correct link” joins two spots produced by the same emitter in different frames; otherwise, a link is “incorrect.” One objective of the linking algorithm is to estimate which links are correct. Links are referred to herein in the format a: i→j This is taken to mean: link α, which begins at spot i and ends at spot j. Links satisfy at least three of the following constraints: (a) links go forward in time, (b) links may not join two spots that are farther apart than some limit (referred to herein as the “search radius”), and (c) links may not join two spots that are temporally separated by more than some limit (referred to herein as the “gap limit”). A spot-link graph is a graph of spots and links for one SMT movie 711. The spots are the vertices and the links are the edges of this graph. Because links go forward in time, the spot-link graph is a directed acyclic graph. A matching is a subset of the links in a spot-link graph such that no two links in this subset begin or end at the same spot. Trajectories 715 are used herein to refer to sequences of contiguous (end-to-end) links in the same matching. Dynamical metrics 730 can be determined using a plurality of trajectories. Such parameters can comprise attributes of a spot that characterize the spot's movement. Such parameters can comprise one or more of velocity, diffusion coefficient, or anomaly parameter(s) for each spot. The dynamical parameter(s) for spot i are herein referred to as θᵢ. The set of dynamical parameters for all spots in a spot-link graph are herein referred to as Θ.

Separate from, and in some variations in parallel with, the processing of SMT movies 711, segmentation movies 708 can undergo segmentation, which generates one or more masks 720. The masks can be of various categories, including but not limited to, cell nuclei, cell cytoplasm, and/or extraneous masks, which are further described in FIG. 8.

Instance masks are individual segmented objects (e.g., one cell, one nucleus, one mitochondrion). A FOV 610 may contain any number of instance masks for one mask category. Semantic masks are the union of all instance masks corresponding to one type of mask category for one FOV (e.g., all cells, all nuclei, or all mitochondria for one FOV, etc.). The extraneous masks can contain parts of the non-SMT movie 708 that are excluded from any downstream data analysis. For example, these extraneous masks could correspond to parts of the non-SMT movie 708 that are out of focus or that contain auto fluorescent cell debris that prevents accurate tracking. During segmentation, molecules within the segmentation movies 708 can be assigned to one or more masks. Image metrics 740 can be evaluated from the masked molecules such as cell health, focus quality, or the like.

Experiment information such as the dynamical metrics 730, the image metrics 740, and any data from which either metric is derived (e.g., segmentation information) can be provided to a data repository 770 for storage. Such data repository 770 can store, for example, any results of experiment 602 such as the dynamical metrics 730, image metrics 740, and/or any data from which either metric is derived. Data repository can comprise local persistence and/or dedicated servers accessed locally or by way of the cloud. Data repository 770 can also store metadata associated therewith and/or metadata associated with the experiment specification 704. The experiment information (e.g., results and metadata from historical experiments, etc.) can be provided to data repository 770 via a repository application program interface (API) 750. The repository API 750 can also interface with a web-based graphical user interface front end 760 that provides such information for display on clients 702.

In some variations, segmentation information can be used to identify subcellular compartments such as nuclei, nucleoli, cytoplasm, and the like. Segmentation information can also be used to distinguish one cell from another. Segmentation information can be stored in a specific format (e.g., a multi-image file format such as TIFF, etc.).

Example dynamical metrics 730 can also include state arrays. State arrays are a framework for learning interpretable dynamical models from SMT trajectories, and can be used for gaining additional insight into the movement of a target protein and where in the cell that movement occurs. In some variations, state arrays can be generated/populated using the segmentation information. The outputs for state arrays can be returned at the subcellular compartment level, allowing scientists to distinguish dynamics in different subcellular compartments. Additionally, state arrays can be computed on each individual subcellular compartment (e.g., per nucleus).

To facilitate data access by applications, including but not limited to state arrays, processed SMT data may be stored in a format that permits (a) representation of processed trajectories and associated attributes such as SNR and spot shape characteristics for each SMT movie, (b) representation of mask objects, including mask category (e.g., each mask object's associated subcellular organelle, etc.), (c) association of trajectories with mask objects (such as the cell nucleus in which each trajectory was observed), and (d) association of all SMT movies with metadata relevant to the original experiment, such as compound treatments, acquisition times, and imaging system name. Formats (a) and (c) can be a

Protocol Buffer schema defining a storage format for trajectories along with associated mask objects. Format (b) can be a specialized image file format that includes the mask objects to which each pixel in an FOV belongs. Format (d) may be a PostgreSQL database that records all captured experiments/movies. As a client of processed SMT data, state arrays can draw on these data schemas to report dynamic characteristics of trajectories on a per-mask category or per-mask object basis.

FIG. 8 is a plurality of images 800 illustrating differences between mask categories and instance or semantic masks. As previously discussed, non-SMT movies or segmentation movies can be assigned to a plurality of categories. Such categories can include cell nuclei (e.g., Category A), cell cytoplasm (e.g., Category B), and/or extraneous masks (e.g., Category C). Unique, individual masks can be applied to biological samples. For example, image 810 is of a unique, individual instance mask applied to a cell nucleus (e.g., Category A). Image 812 is of a unique, individual instance mask applied to a cell cytoplasm (e.g., Category B). Image 820 illustrates multiple instance masks applied to one or more nuclei, with individual colors representing a different unique, individual instance mask. Image 822 illustrates multiple masks applied to one or more cytoplasms, with individual colors representing a different, unique individual instance mask. Image 830 illustrates a semantic mask, which is the union of all instance masks, applied to one or more nuclei. Image 832 illustrates a semantic mask applied to one or more cytoplasms.

FIG. 9 illustrates an example computer-implemented environment 900 where an imaging system 910 can interact with a computing architecture to perform the various algorithms described herein. As shown in FIG. 9, the imaging system 910 can interface with one or more clients 950 (e.g., clients 702 via a web application having a graphical user interface such). The one or more clients 950 can interface with one or more servers 920 accessible through the network(s) 930. The one or more clients 950 can host a frame grabber that captures images from a camera (e.g., movies 612). Those images can be temporarily stored on the one or more clients 950 and periodically transferred to the one or more servers 920 for remote storage via network 930. The one or more servers 920 can also contain or have access to one or more data stores 940 for storing data collected and/or extracted from a sample by imaging system 910. In some variations, the network 930 may include or interface with one or more network storage arrays 960 for storing data such as the captured images (e.g., movies 612).

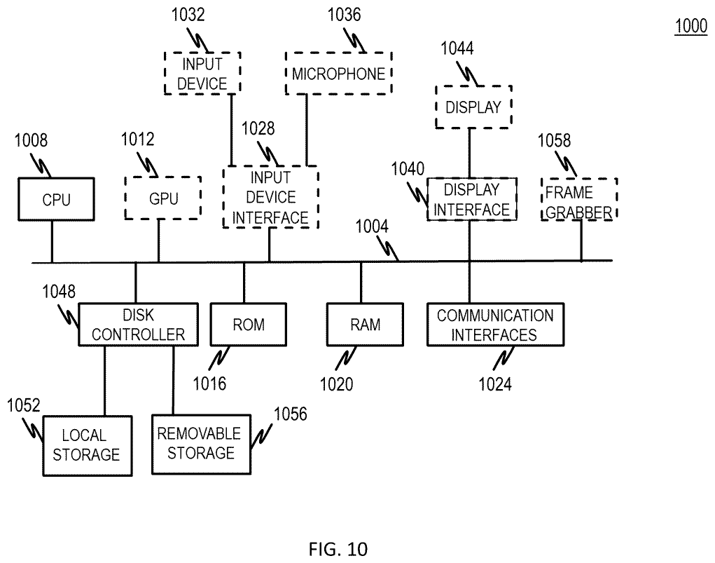

FIG. 10 is a diagram 1000 illustrating a sample computing device architecture for implementing various aspects described herein. In some variations, the sample computing device architecture can be that of client(s) 950 and/or of server(s) 920 and some components described in relation to diagram 1000 may be optional for the client(s) 950 and/or servers(s) 920. A bus 1004 can serve as the information highway interconnecting the other illustrated components of the hardware. A processing system 1008 labeled CPU (central processing unit) (e.g., one or more computer processors/data processors at a given computer or at multiple computers), can perform calculations and logic operations required to execute a program. Optionally or additionally, a processing system 1012 labeled GPU (graphics processing unit) (e.g., one or more computer processors/data processors at a given computer or at multiple computers), can perform calculations and logic operations required to execute a program. A non-transitory processor-readable storage medium, such as read only memory (ROM) 1016 and random access memory (RAM) 1020, can be in communication with the processing system 1008 and/or processing system 1012 and can include one or more programming instructions for the operations specified here. Optionally, program instructions can be stored on a non-transitory computer-readable storage medium such as a magnetic disk, optical disk, recordable memory device, flash memory, solid state drive or other physical storage medium.

In one example, a disk controller 1048 can interface with one or more optional removable storage 1056 or local storage 1052 to the system bus 1004. The removable storage 1056 can be external or internal disk drives, or solid state drives, or external hard drives. The local storage 1052 can be internal hard drives and/or memory. As indicated previously, these various examples of removable storage 1056, local storage 1052, and disk controllers 1048 are optional devices. The system bus 1004 can also include at least one communications interface 1024 to allow for communication with external devices either physically connected to the computing system or available externally through a wired or wireless network such as cloud storage and remote services. In some cases, the at least one communications interface 1024 includes or otherwise comprises a network interface.

In some variations, such as for client(s) 950, to provide for interaction with a user, the subject matter described herein can be implemented on a computing device having a display device 1044 (e.g., LCD (liquid crystal display) or LED (light-emitting diode) monitor) for displaying information obtained from the bus 1004 via a display interface 1040 to the user and an input device 1032 such as keyboard and/or a pointing device (e.g., a mouse or a trackball) and/or a touchscreen by which the user can provide input to the computer. Other kinds of input devices 1032 can be used to provide for interaction with a user as well; for example, feedback provided to the user can be any form of sensory feedback (e.g., visual feedback, auditory feedback by way of a microphone 1036, or tactile feedback); and input from the user can be received in any form, including acoustic, speech, or tactile input. The input device 1032 and the microphone 1036 can be coupled to and convey information via the bus 1004 by way of an input device interface 1028. By way of example, input device 1032 may be an imaging system 910 configured with abilities to capture a sequence of images as described herein. A frame grabber 1058 can capture or grab individual frames from analog or digital data encapsulating the sequence of images obtained from the bus 1004. Frame grabber 1058 may include memory that can store individual or multiple frames. Frame grabber 1058 can also provide individual or multiple frames to bus 1004 for further storage on, for example, local storage 1052 and/or removable storage 1056. Other computing devices, such as dedicated servers, can omit one or more of the components described in connection with FIG. 10.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

### 4. SPECIFIC htSMT APPLICATIONS

Many, and perhaps most, pathways that regulate the fundamental biochemistry of cells depend upon the interaction of protein sensors with protein effectors that engage transiently to trigger a change in cell physiology. Although the fundamentals of this process have long been appreciated, biochemical investigation of these protein interactions has typically required in vitro reconstitution or has been interrogated through pull-down assays after cell permeabilization. The htSMT workflow described herein provide a means of visualizing protein movement in large numbers of live cells, and under circumstances where the effect of added compositions, e.g., small molecule inhibitors, can be assessed quantitatively.

With reference to FIG. 1, aspects of the htSMT workflows of the present disclosure include, but are not limited to, (i) sample preparation including reagent handling, (ii) image acquisition using imaging of the samples to generate a series of images and/or videos, (iii) image analysis through processing of these images and video, (iv) storage of information, and (v) provision of insights using the stored information including biological interpretation. With respect to the biological interpretations, the htSMT workflows described herein offer the ability to provide specific insights, as outlined below, depending on the particular workflow employed, e.g., (i) htSMT Screening; (ii) htSMT Binding; and/or (iii) KineticSMT.

In certain embodiments, the workflows of the present disclosure can comprise illuminating a detected field of view in a sample plane disposed within the sample with a light beam to cause fluorescence by a subset of the fluorescent target proteins in the live cells, where the detected field of view has a size of about 50 μm to less than 100 μm in a first dimension by about 50 μm to less than 100 μm in a second dimension. For example, but not by way of limitation, the detected FOV can have a size of about 94 μm in a first dimension by about 94 μm in a second dimension.

In certain embodiments, the workflows of the present disclosure can comprise illuminating a detected field of view in a sample plane disposed within the sample with a light beam to cause fluorescence by a subset of the fluorescent target proteins in the live cells to image a plurality of trajectories. In certain embodiments, the number of trajectories imaged in a detected field of view can be from about 10,000 to about 100,000, e.g., about 20,000 to about 40,000. For example, but not by way of limitation, the number of trajectories imaged in a detected field of view can be from about 10,000 to about 50,000, from about 10,000 to about 40,000, from about 20,000 to about 50,000 or from about 20,000 to about 40,000. In certain embodiments, the number of trajectories imaged in a detected field of view can be up to about 100,000, e.g., up to about 95,000, up to about 90,000, up to about 85,000, up to about 80,000, up to about 75,000, up to about 70,000, up to about 65,000, up to about 60,000, up to about 55,000, up to about 50,000, up to about 45,000, up to about 40,000, up to about 35,000 or up to about 30,000.

In certain embodiments, a detected field of view can include a plurality of cells. In certain embodiments, the number of cells imaged in a detected field of view is related to the size of the cells being imaged. For example, but not by way of limitation, the smaller the size of the cell, the greater the number of cells that can be imaged in a detected field of view. In certain embodiments, depending on the size of the cell being imaged, a detected field of view can include about 1 to about 50 live cells, e.g., can include about 1 to about 45 cells, about 1 to about 40 cells, about 1 to about 35 cells, about 1 to about 30 cells, about 1 to about 25 cells, about 1 to about 20 cells, about 1 to about 15 cells, about 1 to about 10 cells, about 1 to about 5 cells, about 5 to about 40 cells, about 10 to about 40 cells, about 15 to about 40 cells, about 20 to about 40 cells, about 10 to about 35 cells, about 15 to about 35 cells or about 20 to about 30 cells. In certain embodiments, depending on the size of the cell being imaged, a detected field of view can include about 1 to about 40 live cells, e.g., mammalian cells. In certain embodiments, depending on the size of the cell being imaged, a detected field of view can include about 1 to about 30 live cells, e.g., mammalian cells. In certain embodiments, depending on the size of the cell being imaged, a detected field of view can include about 1 to about 20 live cells, e.g., mammalian cells. In certain embodiments, a detected field of view can include up to about 50 live cells, e.g., up to about 45 live cells, up to about 40 live cells, up to about 35 live cells, up to about 30 live cells, up to about 25 live cells or up to about 20 live cells. In certain embodiments, a detected field of view can include up to about 40 live cells. In certain embodiments, a detected field of view can include up to about 30 live cells.

In certain embodiments, the workflows of the present disclosure can include detecting the fluorescence from individual fluorescent target proteins in the plurality of fluorescent target proteins in a detected field of view of the sample plane at a rate of >100 detected FOVs per day, >10,000 detected FOVs per day, or >100,000 detected FOVs per day, where the detected field of view is about 50 μm to less than 100 μm in a first dimension by about 50 μm to less than 100 μm in a second dimension. In certain embodiments, the workflows of the present disclosure can include detecting the fluorescence from individual fluorescent target proteins in the plurality of fluorescent target proteins in a detected field of view of the sample plane at a rate of >100 detected FOVs per day, where the detected field of view is about 50 μm to less than 100 μm in a first dimension by about 50 μm to less than 100 μm in a second dimension. In certain embodiments, the workflows of the present disclosure can include detecting the fluorescence from individual fluorescent target proteins in the plurality of fluorescent target proteins in a detected field of view of the sample plane at a rate of >10,000 detected FOVs per day, where the detected field of view is about 50 μm to less than 100 μm in a first dimension by about 50 μm to less than 100 μm in a second dimension. In certain embodiments, the workflows of the present disclosure can include detecting the fluorescence from individual fluorescent target proteins in the plurality of fluorescent target proteins in a detected field of view of the sample plane at a rate of >100,000 detected FOVs per day, where the detected field of view is about 50 μm to less than 100 μm in a first dimension by about 50 μm to less than 100 μm in a second dimension.

In certain embodiments, the workflows of the present disclosure can comprise illuminating a detected field of view in a sample plane disposed within the sample with a light beam to cause fluorescence by a subset of the fluorescent target proteins in the live cells, where the detected field of view has a size of about 50 μm to less than 100 μm in a first dimension by about 50 μm to less than 100 μm in a second dimension and wherein up to 70% of the detected field of view achieves sufficient laser illumination for tracking protein movement. In certain embodiments, the detected field of view has a size of about 50 μm to less than 100 μm in a first dimension by about 50 μm to less than 100 μm in a second dimension and wherein up to 60% of the detected field of view achieves sufficient laser illumination for tracking protein movement. In certain embodiments, the detected field of view has a size of about 50 μm to less than 100 μm in a first dimension by about 50 μm to less than 100 μm in a second dimension and wherein up to 50% of the detected field of view achieves sufficient laser illumination for tracking protein movement. In certain embodiments, the detected field of view has a size of about 50 μm to less than 100 μm in a first dimension by about 50 μm to less than 100 μm in a second dimension and wherein up to 40% of the detected field of view achieves sufficient laser illumination for tracking protein movement. In certain embodiments, the detected field of view has a size of about 50 μm to less than 100 μm in a first dimension by about 50 μm to less than 100μm in a second dimension and wherein up to 30% of the detected field of view achieves sufficient laser illumination for tracking protein movement. In certain embodiments, the detected field of view has a size of about 50 μm to less than 100 μm in a first dimension by about 50 μm to less than 100 μm in a second dimension and wherein up to 20% of the detected field of view achieves sufficient laser illumination for tracking protein movement. In certain embodiments, the detected field of view has a size of about 50 μm to less than 100 μm in a first dimension by about 50 μm to less than 100 μm in a second dimension and wherein up to 10% of the detected field of view achieves sufficient laser illumination for tracking protein movement.

In certain embodiments, the workflows of the present disclosure can include determining a change in the movement of the fluorescently labeled target protein in the presence of the compound. For example, but not by way of limitation, the average change in movement of the fluorescent target protein in the presence of a compound is at least 1%, at least 5%, at least 10%, relative to the change observed in the absence of the compound. In certain embodiments, the average change in movement of the fluorescent target protein in the presence of a compound is about 1% to about 5%. In certain embodiments, the average change in movement of the fluorescent target protein in the presence of a compound is about 1% to about 10%.

In certain embodiments, exemplary htSMT workflows comprise individual strategies described above as well as combinations of these strategies where two or more of the strategic requirements are combined.

### 4.1 htSMT Screening

In certain implementations of the htSMT workflows described herein, the systems and methods are adapted to interrogate the ability of one or more compositions, e.g., “test” compounds, to impact the SMT profile associated with a fluorescent protein. For example, such htSMT workflow will screen for changes in the SMT profile, e.g., either an increase or a decrease in movement of the protein of interest, in the presence of the composition relative to that SMT profile in the absence of the composition. It will be appreciated that higher-order comparisons can also be made with where compounds are multiplexed, including where multiple proteins are fluorescent. Moreover, as outlined above, the htSMT screening strategies described herein are equally applicable to screening of the SMT profiles associated with fluorescent compounds, e.g., compounds that are naturally fluorescent or those that have been modified to fluoresce or are linked to a fluorophore.

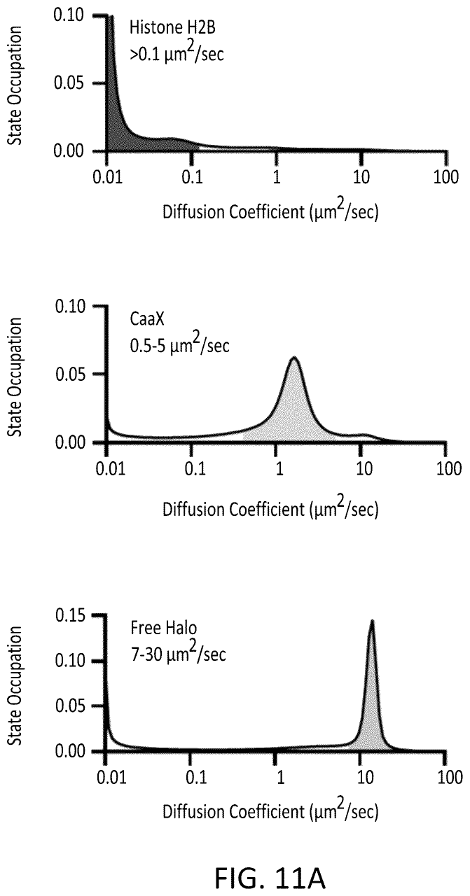

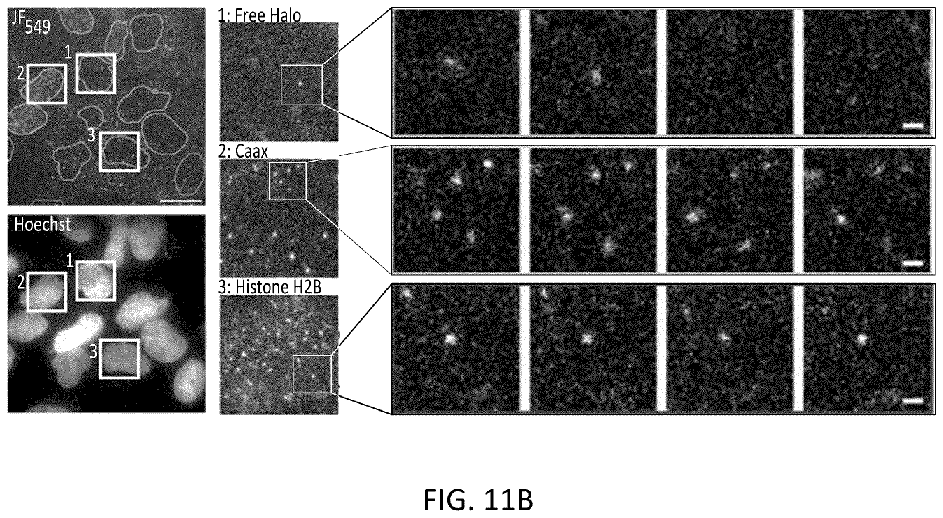

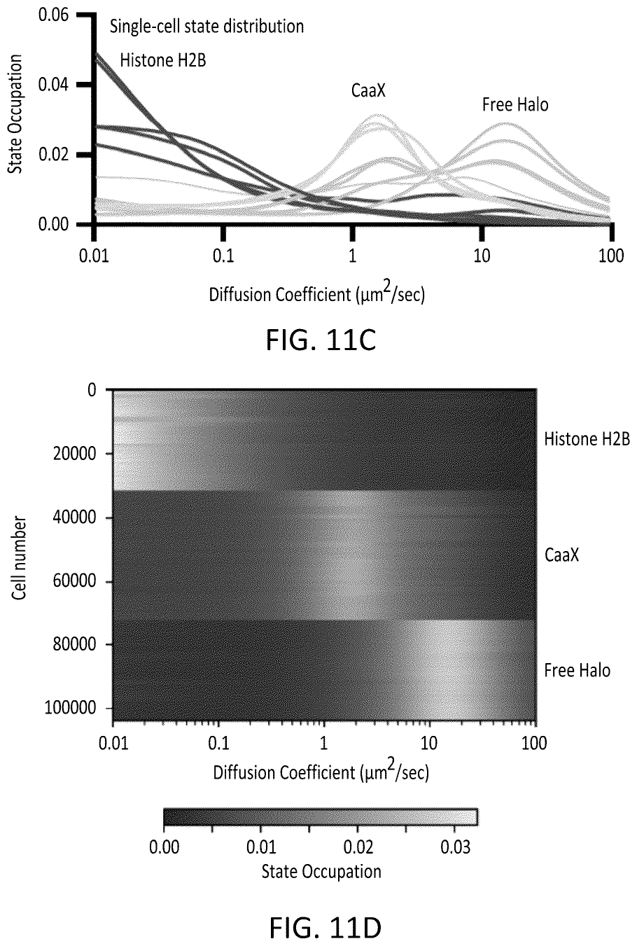

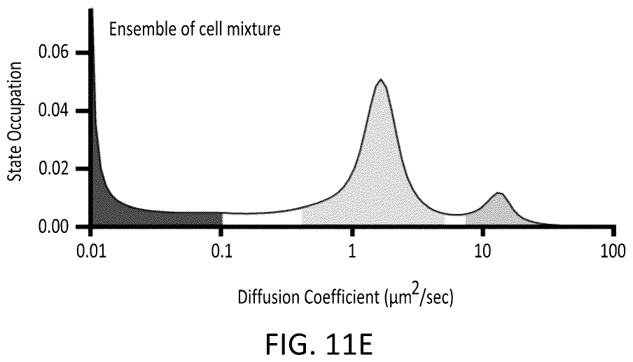

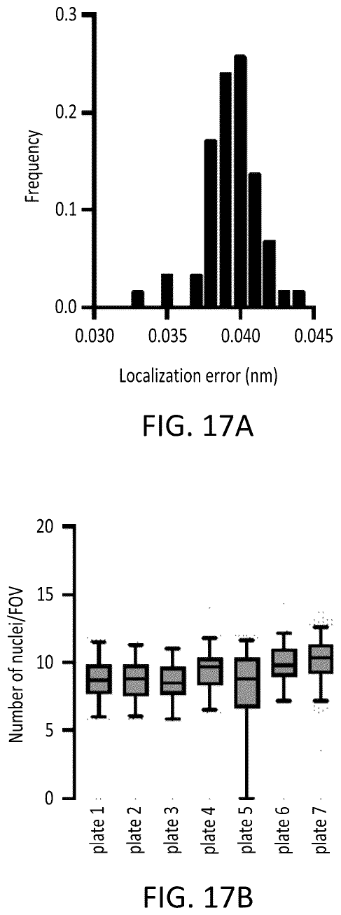

Underlying such htSMT screening strategies is the ability of the htSMT workflows described herein to extract accurate movement data at scale. This ability is evidenced by the exemplary results presented in FIGS. 11A-11E. In the experiments reported in FIGS. 11A-11E, 384-well plates were employed where free Halo, Halo-CaaX, and H2B-Halo cell lines were mixed in equal proportions in each well. Imaging with a 94 μm by 94 μm field-of-view (FOV), achieved an average of 10 nuclei simultaneously (FIG. 11B, FIG. 17B), enough that most FOVs contained cells from each cell line. To limit ambiguity in cell assignment, only the tracks that fell within nuclear-segmented regions were considered. The probability distribution of movement cleanly distinguishes between the three cell types (FIG. 11C). More importantly, by looking at the single-cell state distribution profiles of 103,757 cells from five separate 384-well plates, grouped by their distribution profile, highly consistent estimates of protein movement at the single cell level were recovered (FIG. 11D). Moreover, by combining data from multiple cells, the expected distribution can be provided (FIG. 11E). In addition, combining data derived from many cells makes SpotOn or State Array analysis possible, where as few as 10³ trajectories permit satisfactory inference of the underlying states. Only a few seconds of imaging per FOV yield enough trajectories (>10,000) to accurately estimate protein movement, bringing the overall throughput of the platform to >90,000 FOVs per day, a rate of data acquisition that enables drug screening on a feasible timescale.

Equipped with an htSMT system capable of measuring protein movement, the following disclosure establishes that measurements of protein movement can be used to characterize proteins functionally. For example, using steroid hormone receptors (SHRs), which transition between inactive and active states via ligand binding (FIG. 12A), the htSMT workflow described herein can capture functionally relevant differences. Specifically, in the absence of hormone, four SHRs exhibit similar movement profiles: a small immobile fraction and a large freely moving fraction with a 3.4-4.3 μm²/sec average diffusion coefficient (FIG. 12B). No correlation between movement and protein size was observed, however, highlighting the differences between cellular protein movement versus purified systems. Highlighting the selectivity and sensitivity of htSMT, upon addition of agonist, a dramatic increase in immobile trajectories is observed, which is attributable to chromatin binding. As illustrated in FIG. 12B, the bound fraction (f_{bound}) is defined for each SHR as the fraction of tracks moving less than 0.1 μm²/sec. Consistent with previous findings, some SHRs had a higher proportion of bound molecules than others, both with and without ligand. The ligand-induced effect is most pronounced for ER, with 34% bound in basal conditions and 87% bound after estradiol treatment (FIG. 12B).

Supporting their use as a representative target family for htSMT analysis, SHRs are highly selective for their cognate agonists in biochemical binding assays, which was confirmed by measuring the dose-dependent change in movement as a function of agonist concentration. The maximal increase in f_{bound} (FIG. 12C) and decrease in free diffusion coefficient (D_{free}; FIG. 12D) differed between SHRs. The dose titration curves also showed variable potencies (EC50) for each SHR/hormone pair, with ER-estradiol being both the most potent and most selective pair. Thus, screening via htSMT can precisely and accurately differentiate the ligand/target specificity directly within the living cellular environment.

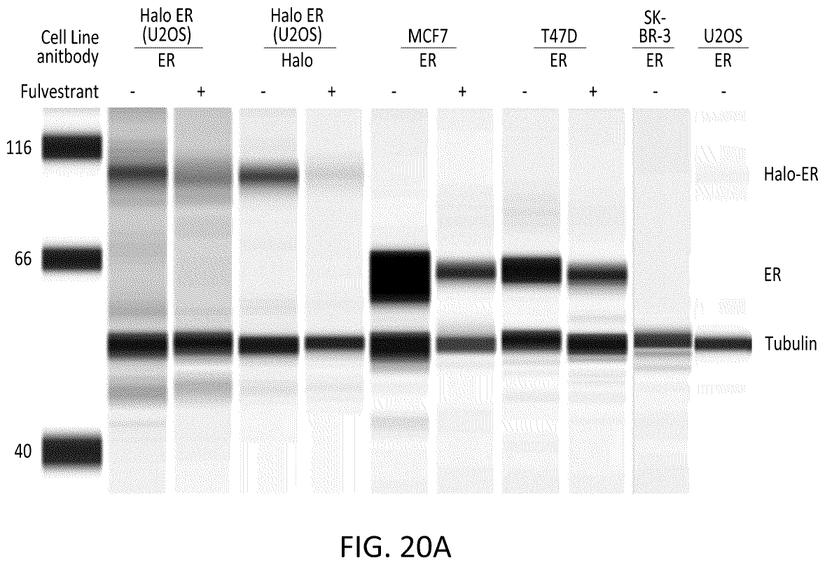

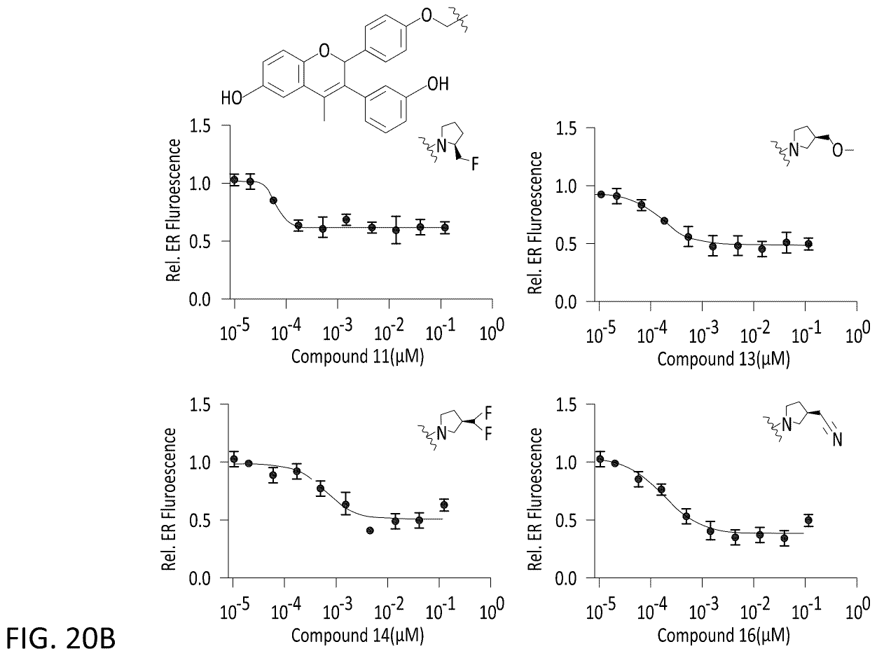

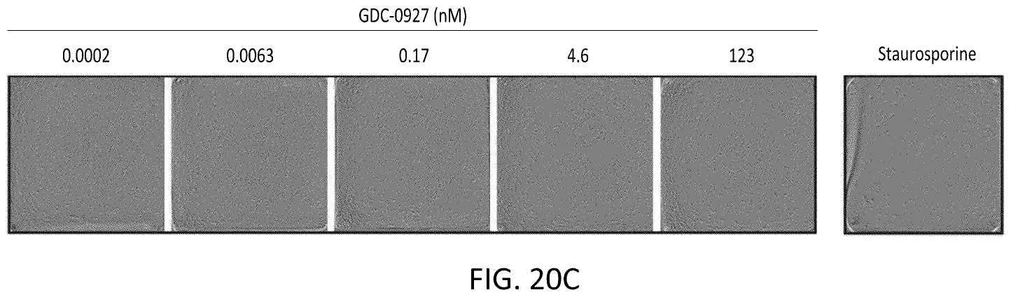

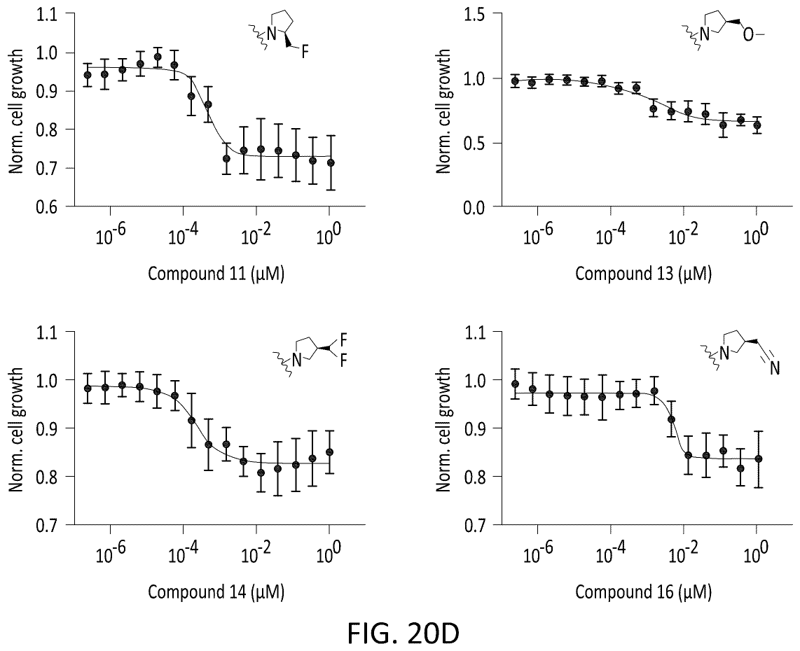

In another example of an htSMT screening assay, next-generation ER degraders like GDC-0927, AZD9833, and GDC-9545 were optimized to enhance degradation of ER. Compound-induced changes in protein persistence, e.g., ER degradation, were indeed observed both in established breast cancer model lines and the U2OS expression system (FIG. 15A, FIG. 20A). Structural analogs of GDC-0927 have been reported and optimized for ER degradation, however the correlation between ER degradation and cell proliferation is poor (FIG. 15B, FIG. 20B-20D). By measuring protein persistence, however, more precise measurements of inhibitory activity can be obtained than can be achieved by assessing protein degradation. The potency and maximal effect of structural analogues of GDC-0927 was determined using htSMT. Overall, these analogues exhibited a potency range of 15 pM to 12 nM and increased ER f_{bound} by 0.4 to 0.56 (FIG. 15C). Small changes in the chemical structure produced measurable changes in both compound potency and maximal efficacy as determined using htSMT.

The potencies of GDC-0927 and analogues determined either via ER degradation or htSMT were compared to the ability of each of these compounds to block estrogen-induced breast cancer cell proliferation. Potency assessed by ER degradation was not a good predictor of potency in the cell proliferation assay (FIG. 15C). By contrast, SMT measurements of f_{bound} strongly correlate with cell viability (FIG. 15D; R² of 0.83 for T47d and 0.84 for MCF7). Intriguingly, SMT EC₅₀ values were on average 10-fold lower than those observed in the cell growth assay, indicating that SMT is sensitive enough to permit the selection of chemical series which would not show effects in other cellular assays. This correlation between effects on protein movement (e.g., f_{bound}) and protein function (suppression of cell proliferation) coupled with the throughput of the SMT system make this an attractive approach for the identification of protein modulators with novel properties.

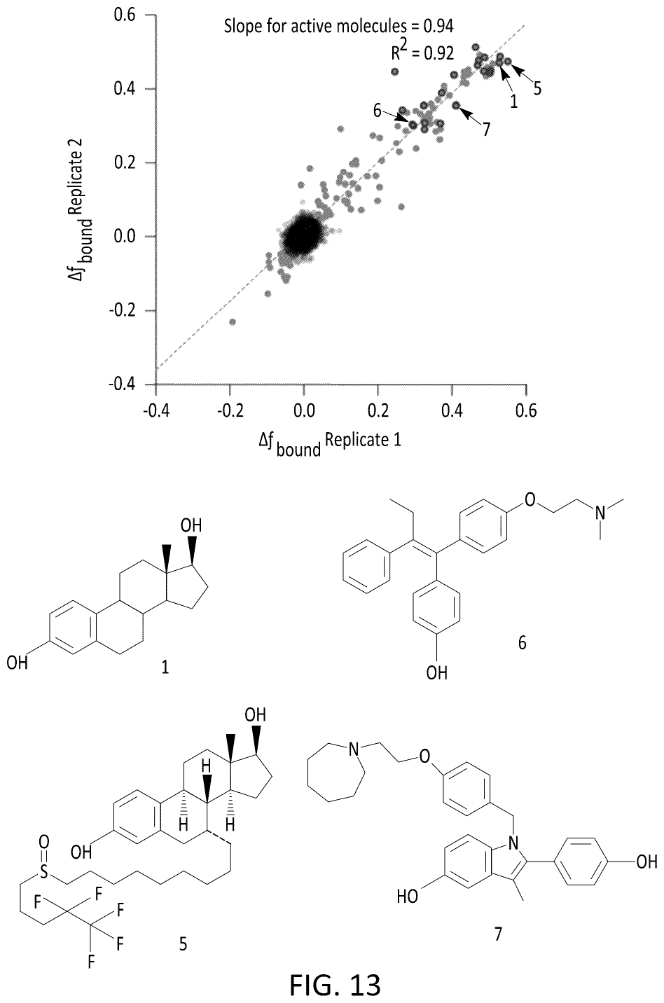

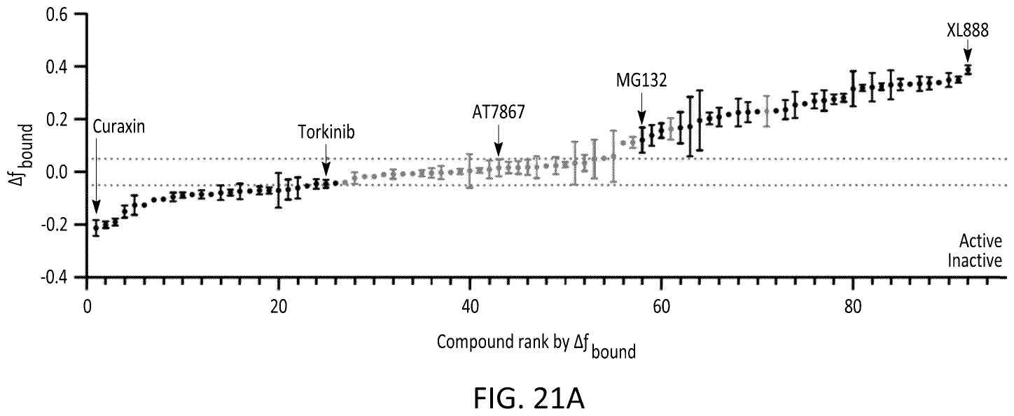

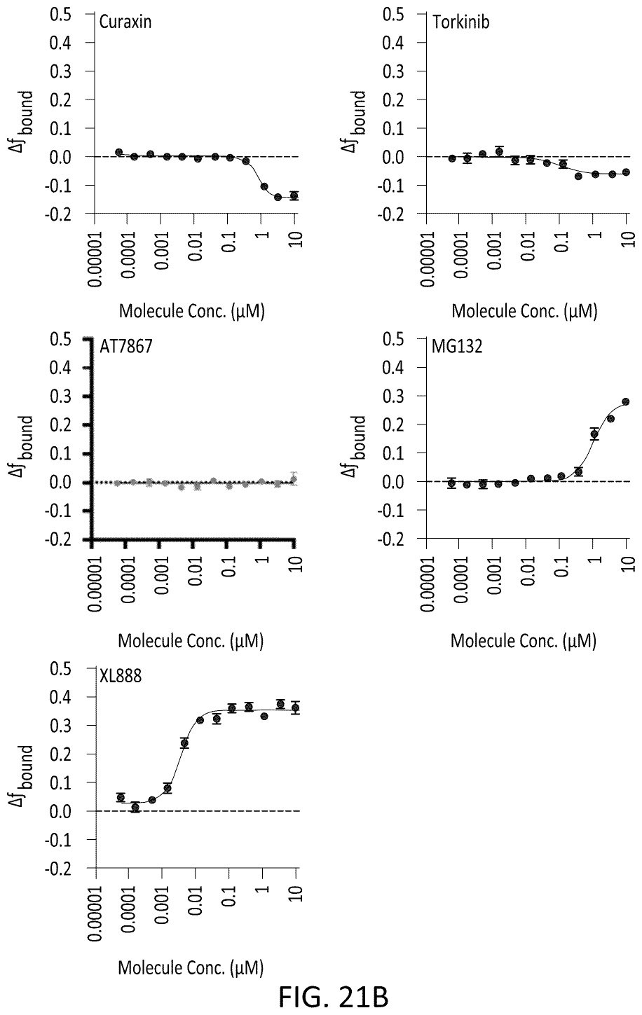

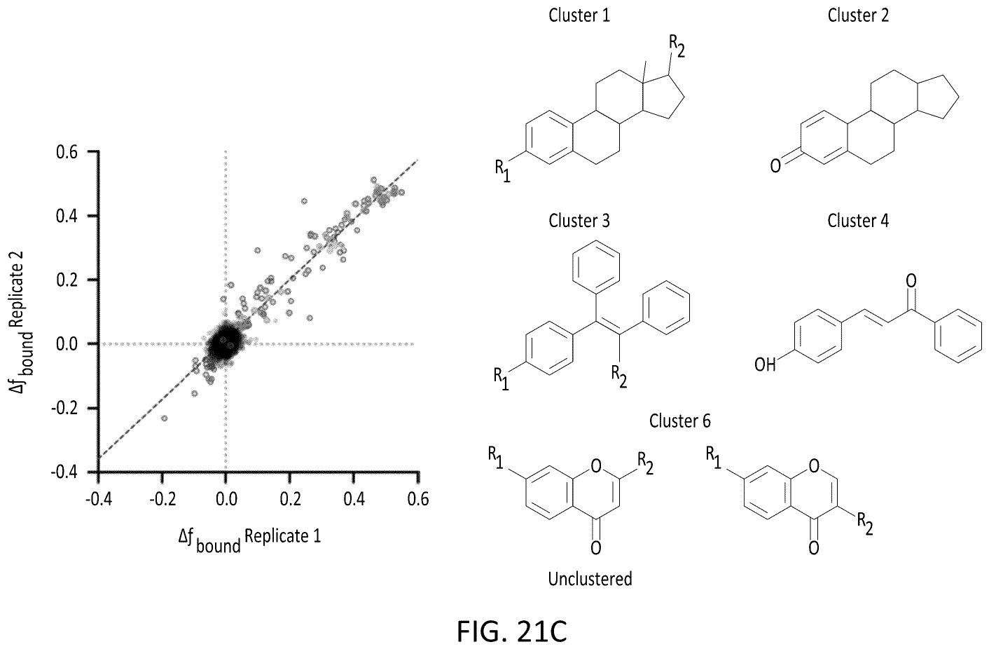

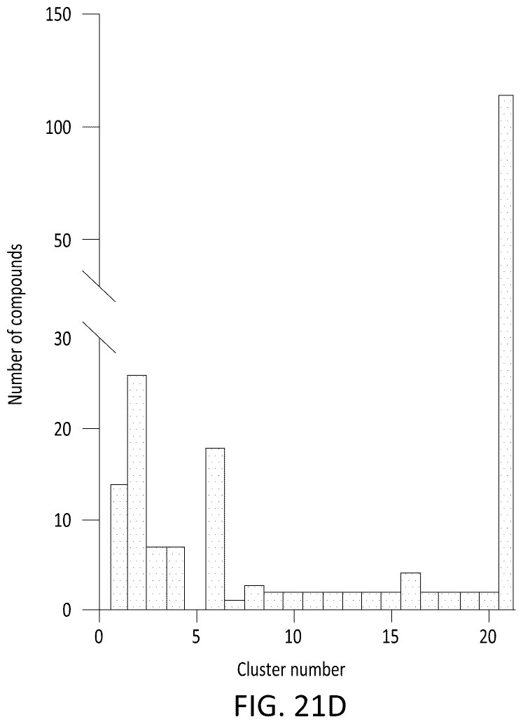

In addition to known ER active modulators, many other compounds present in the bioactive library tested provoked easily measurable changes in f_{bound}. To define a threshold for calling a molecule from the screen “active”, 92 compounds with different magnitudes of change in f_{bound} were selected to retest in a dose titration (FIG. 21A, 21B). A 5% change in f_{bound} was sufficient to reproducibly distinguish active compounds. Using this approach, 239 compounds were identified in the bioactive library that affected the ER mobility (FIG. 13). Among these compounds, the correlation between the two screen replicates was high (R²=0.92) and the level of activity was reproducible (the slope for active molecules was 0.94). Some active compounds could be clustered based on scaffold homology, but most clusters consisted of one or only a few members (FIG. 21C, 21D). Structural clustering was employed to identify known ER modulators where the vendor-provided annotation was poorly defined. These results demonstrate that htSMT is reproducible and robust when screening large collections of molecules.

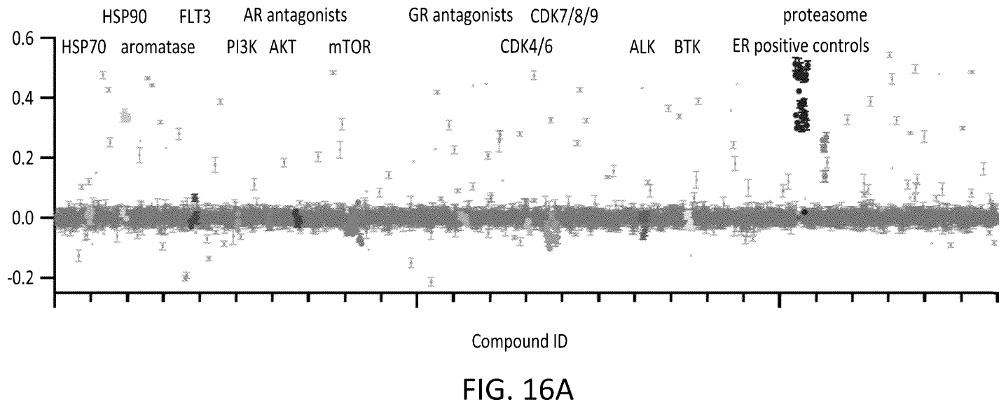

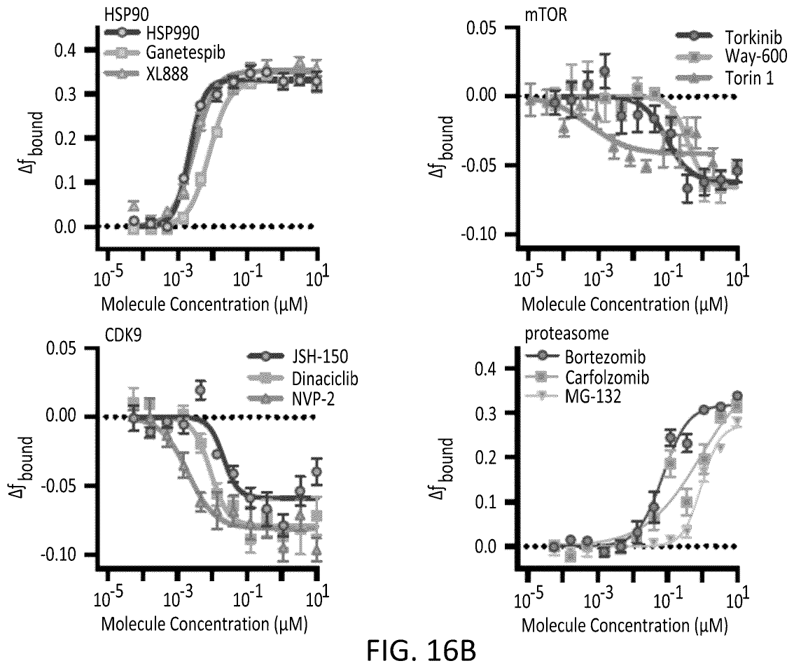

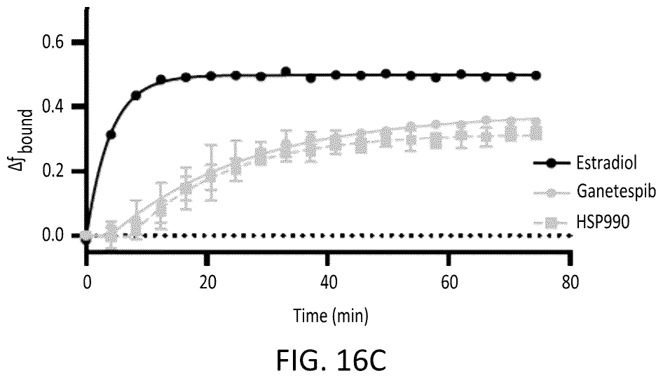

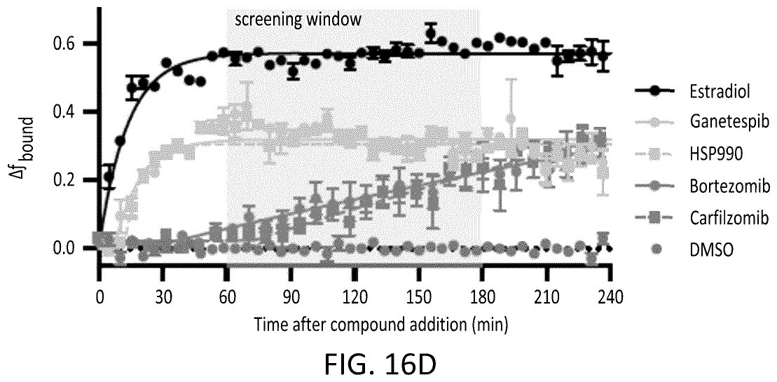

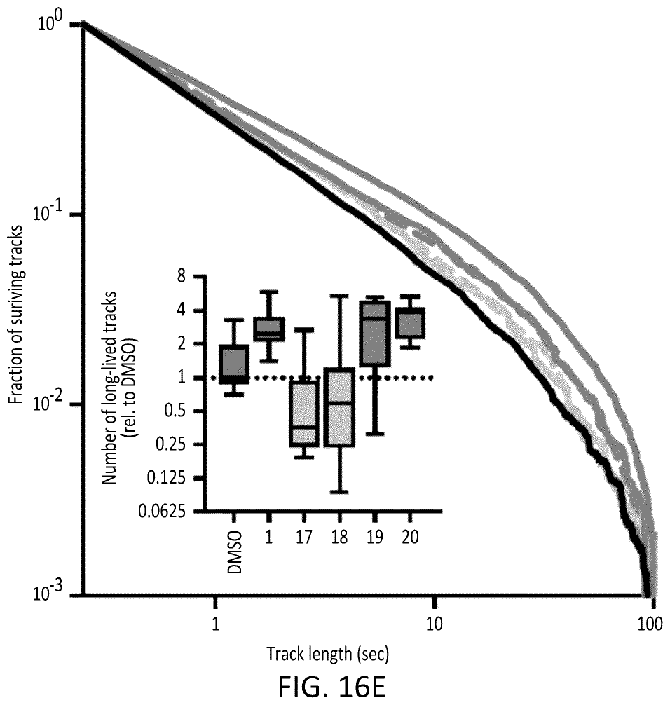

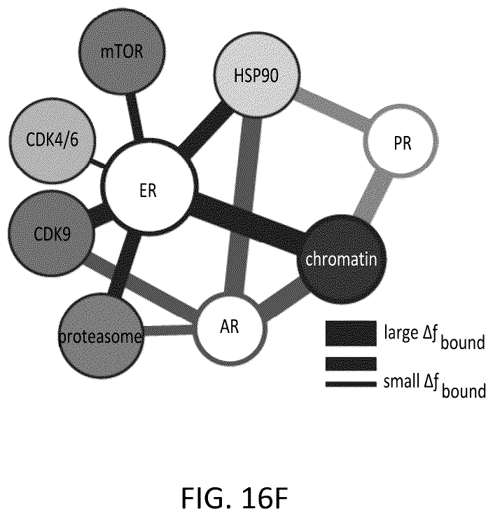

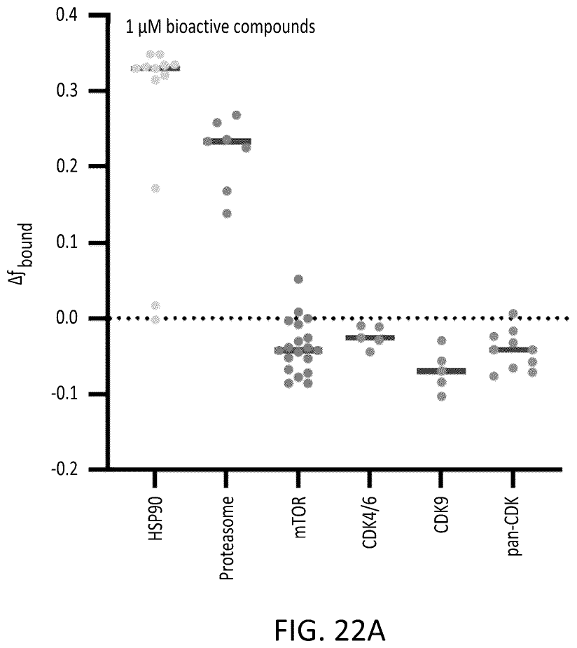

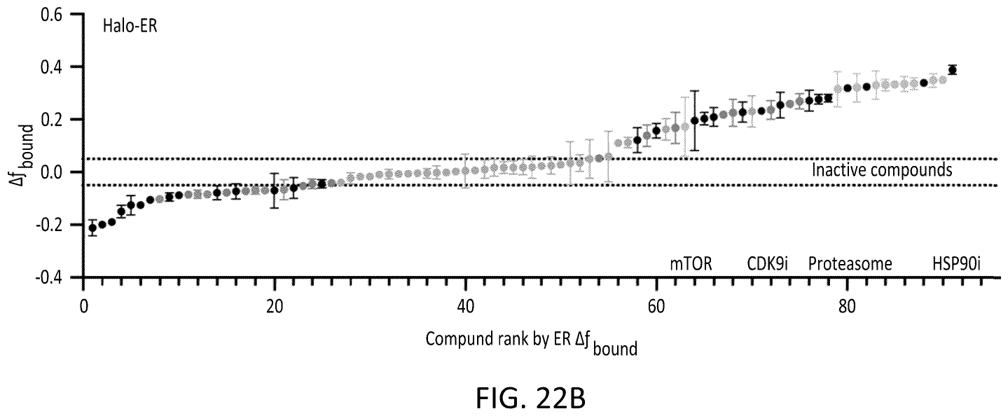

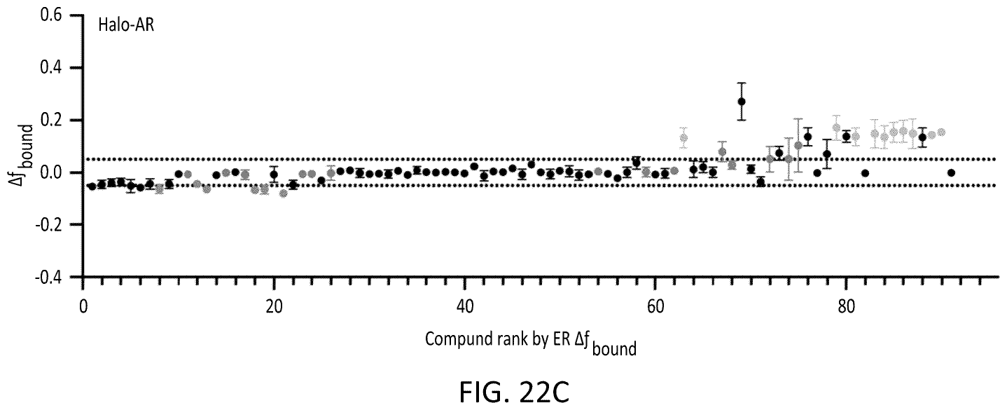

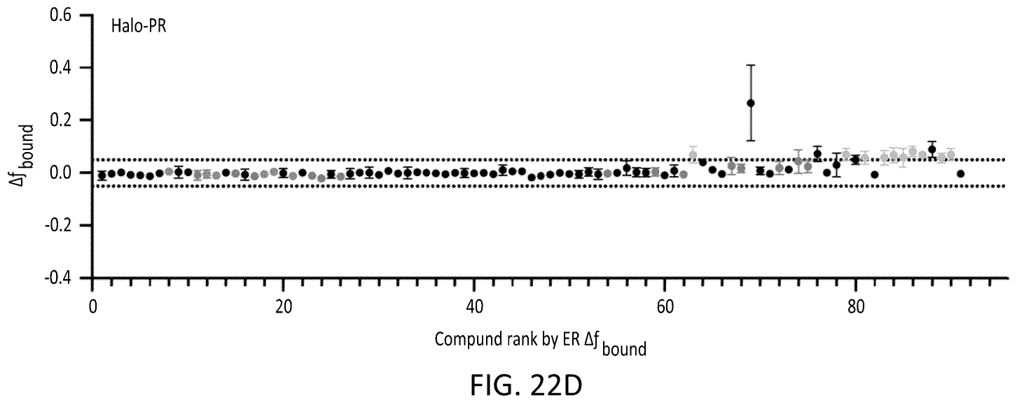

Most active molecules from the screen were not structurally related to steroids (FIG. 21C, 21D). On the other hand, many compounds could be grouped based on their reported biological targets or pathways (FIG. 16A, FIG. 22A, 22B). For example, heat shock protein (HSP) and proteasome inhibitors consistently increased f_{bound}, whereas cyclin dependent kinase (CDK) and mTOR inhibitors decreased f_{bound}. Though many CDK inhibitors lack within-family specificity (FIG. 22A, pan-CDK), CDK9-specific inhibitors were found to more strongly affected ER movement than did CDK4/6-specific inhibitors. Furthermore, as with selective AR and GR antagonists, inhibitors targeting ALK, BTK, and FLT3 kinases that have not been shown to interact with ER have no impact on ER movement when assessed using SMT (FIG. 16A).

For the inhibitors of cellular pathways that were identified, a dose titration was used to better characterize the effect of each on ER movement. Potencies ranged from the sub-nanomolar to low micromolar (FIG. 16B), similar to the reported potencies of these compounds against their cellular targets. Additionally, these molecules were tested against AR (FIG. 22C) and PR (FIG. 22D). Each SHR differed meaningfully from the others in terms of the response to compounds identified through an ER-focused screening effort. Again, the magnitude of ER SMT effect was largely consistent within a target class (FIG. 16B, FIG. 22A-22D). The finding that structurally distinct compounds exhibited similar effects based on their biological targets favors the view that these biological targets must themselves interact with ER, and that the compounds therefore affect ER movement indirectly. HSP90 is a chaperone for many proteins, including SHRs. In the canonical model, hormone binding releases the SHR-HSP90 complex. Indeed, HSP90 inhibitors increased f_{bound} for ER, AR, and PR, consistent with one function of the chaperone being to adjust the equilibrium of SHR binding to chromatin (FIG. 22B-22D). Proteasome inhibition also leads to ER immobilization on chromatin, which aligns with the results obtained in the htSMT screen of bioactive compounds. ER has been shown to be phosphorylated by CDK, Src, or GSK-3 through MAPK and PI3K/AKT signaling pathways, and therefore inhibition of these pathways would reasonably be expected to affect ER movement measured using SMT. While CDK inhibition led to an increase in ER mobility, inhibition of PI3K, AKT, or other upstream kinases showed no effect (FIG. 16A).

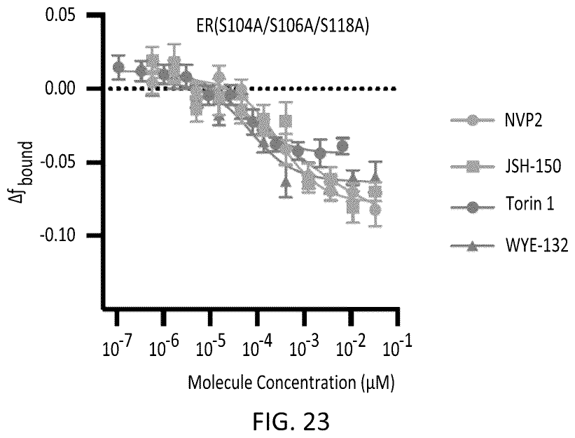

Interestingly, SMT movement of an ER triple point mutant engineered to lack previously defined phosphorylation sites important for transactivation (S104A/S106A/S118A) were affected by CDK and mTOR pathway inhibitors (FIG. 23), which indicates that additional phosphorylation sites can mediate the effects of CDK9 and PI3K/AKT signaling, or that other molecular targets of CDK and PI3K/AKT can act indirectly to alter the movement of ER. The change in ER protein movement for characterized pathway inhibitors such as those targeting CDK and mTOR is subtle but consistent across compounds, indicating biological meaning in these observations and highlight the need for accurate and precise SMT measurements. Hence the htSMT techniques described herein offer avenues to provide comprehensive pathway interaction information.

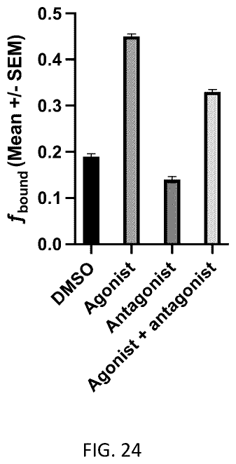

Further evidencing the fact that monitoring changes in binding via changes in target movement can support the identification of pharmacologically-relevant compounds, known agonists and antagonists of AR were assayed (FIG. 24). While AR agonists also increase f_{bound}, antagonists of AR cause a decrease in f_{bound} both in single treatment as well as when co-administered with the AR agonist. Hence, both increases in f_{bound} and decreases in f_{bound} can be useful in identifying mechanistically distinct mechanisms for pharmacological interaction with the fluorescent target protein under observation.

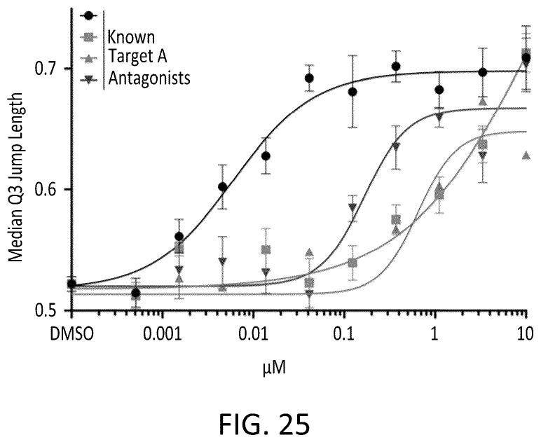

In addition to changes in movement associated with chromatin binding, htSMT can also be used to monitor and identify pharmacological compounds that disrupt or enhance protein-protein interactions and protein conformational changes. One such example is the disruption of a ubiquination process that is dependent on a series of protein-protein interactions. By using known antagonists that disrupt the underlying protein-protein interactions, large changes in the movement of one of the proteins involved in the interaction (Target A) are produced upon complex disruption, indicative of that protein being more freely moving (FIG. 25). Similarly, htSMT can be used to interrogate protein conformational changes and screen for compounds that modulate targets allosterically. Known ATP-competitive and allosteric inhibitors of multiple receptor tyrosine kinases, e.g., Target B and Target C, provoke significant changes in protein movement. Moreover, htSMT is e exquisitely sensitive to allosteric inhibition, resulting in movement changes that are 4-8 fold higher in magnitude than enzymatic inhibitors (FIG. 26). Thus, the utility of htSMT can meaningfully interrogate both protein-chromatin and protein-protein interactions.

### 4.2 htSMT Binding

In certain implementations of the htSMT workflows described herein, once a compound has been identified as increasing the static binding of a target (e.g., the static binding of ER to chromatin in the presence of the compound) which is referenced herein in certain instances as increasing f_{bound}, a second assay can be performed to obtain more detail as to the nature of the target's static binding. For example, the systems and methods described herein can be adapted to discriminate between recovery after exposure to the compound that is driven by an increase in residence time of the target to its binding partner (i.e., decreasing k*_{off}).

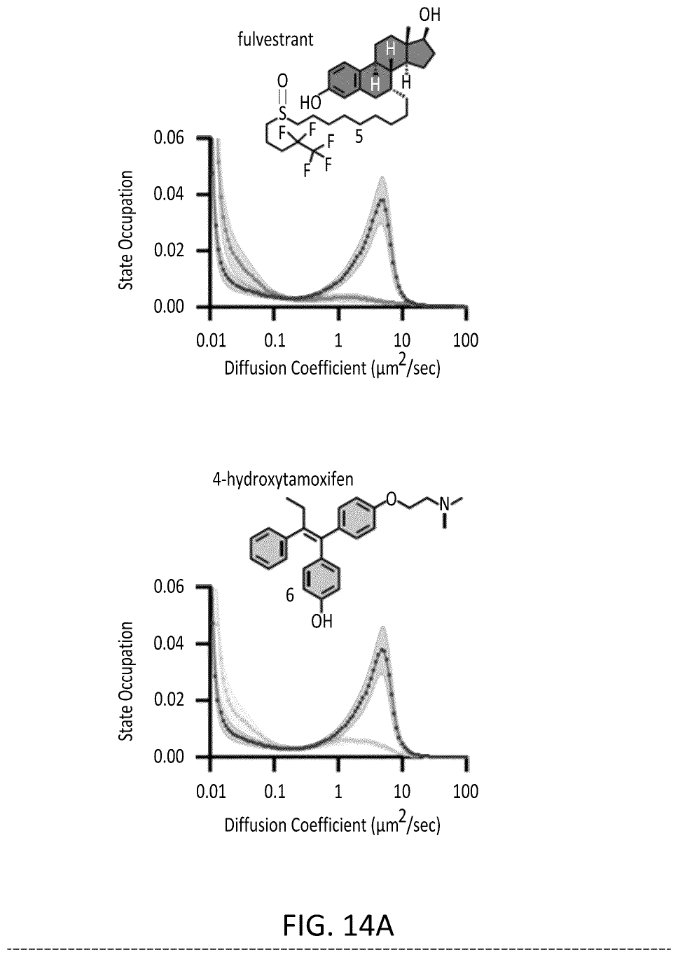

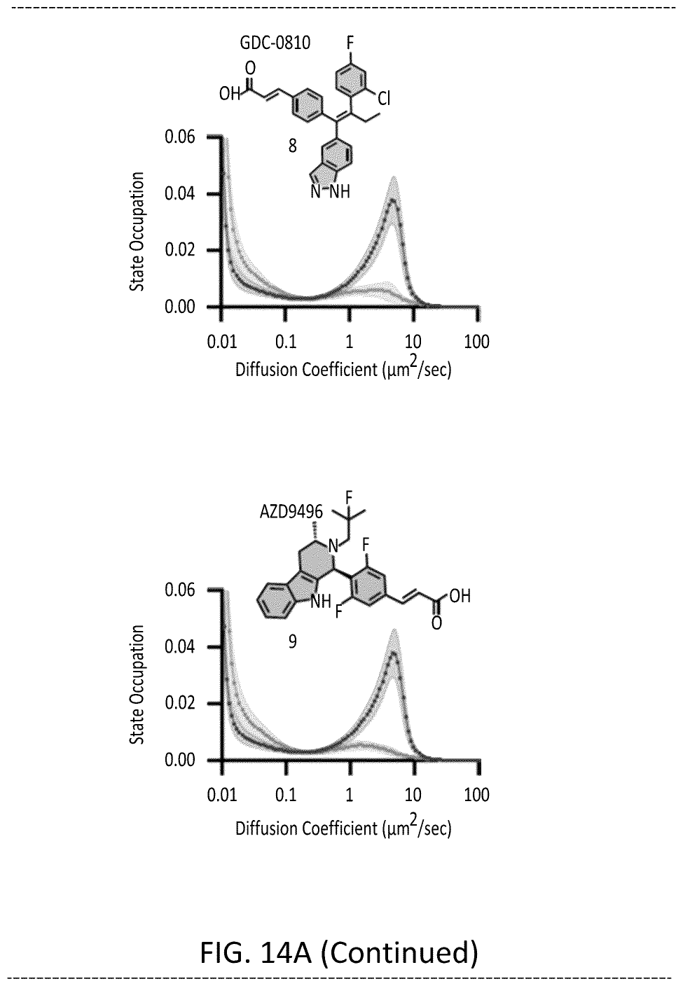

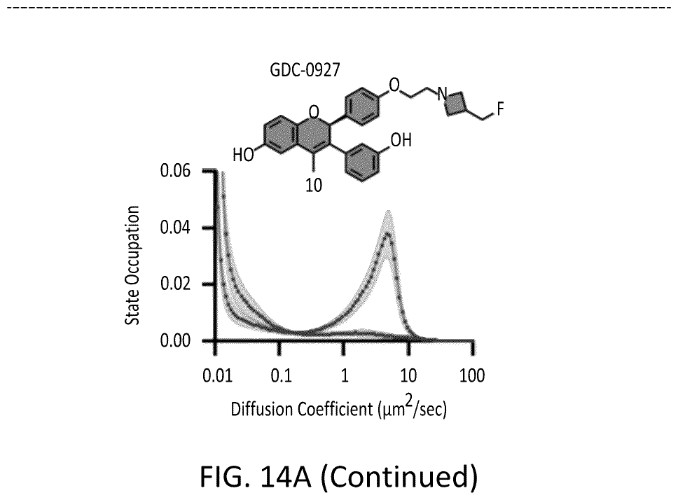

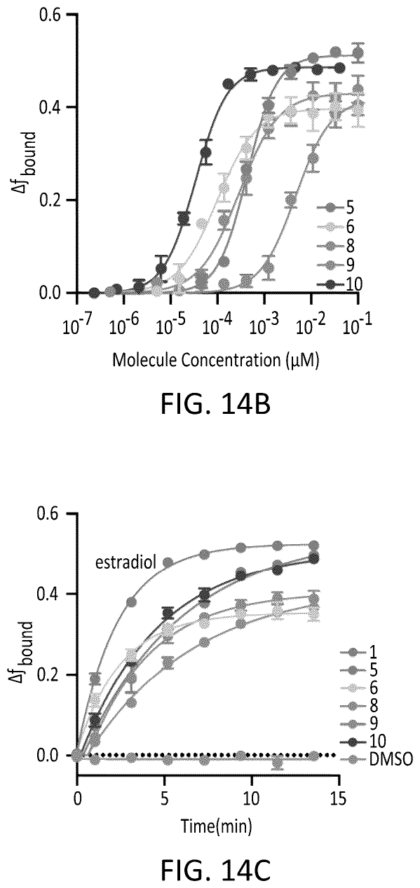

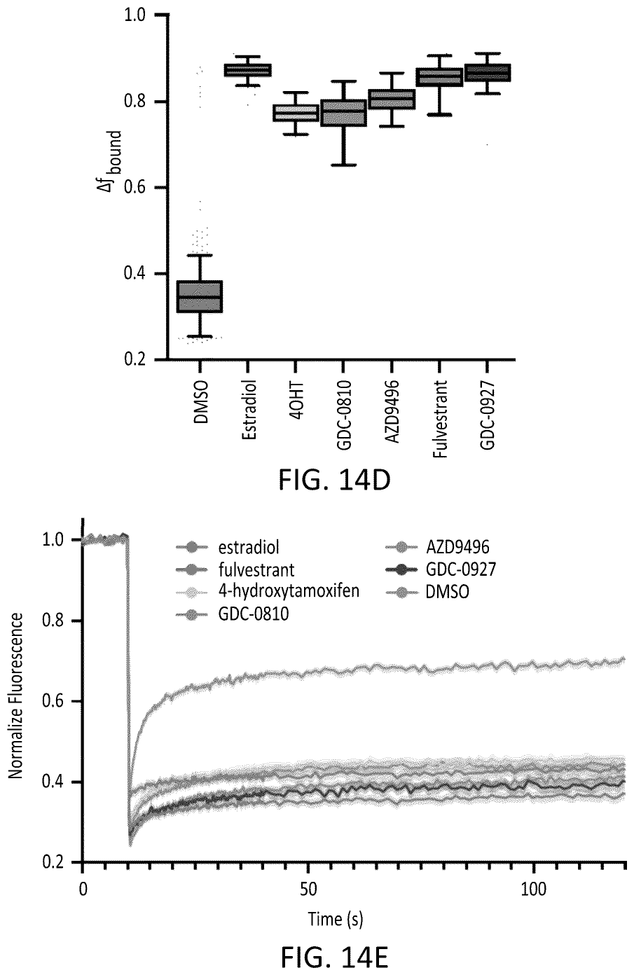

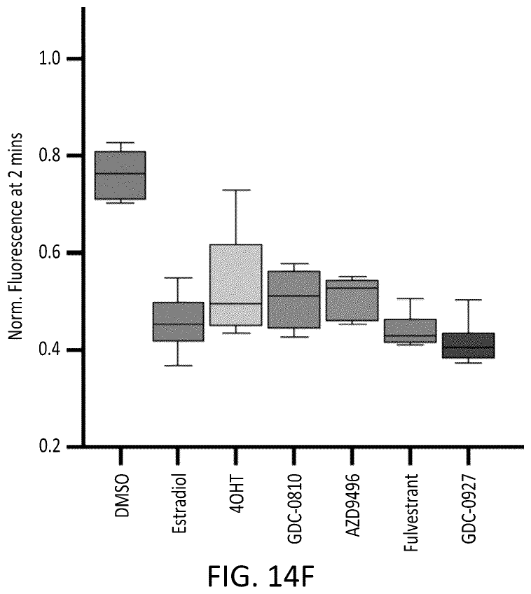

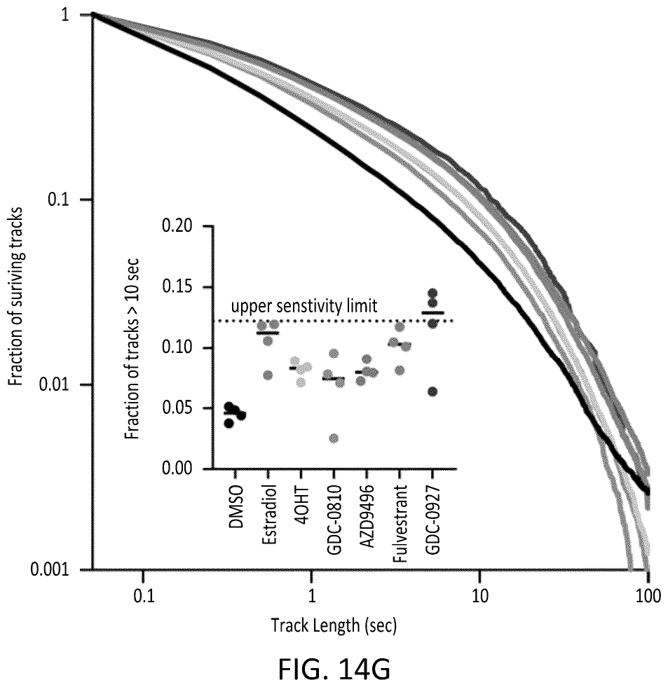

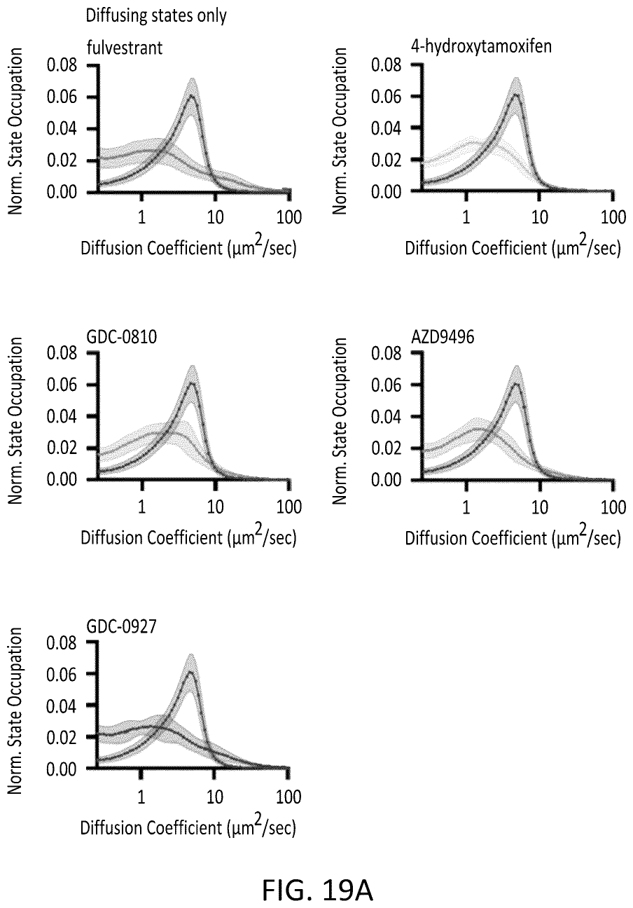

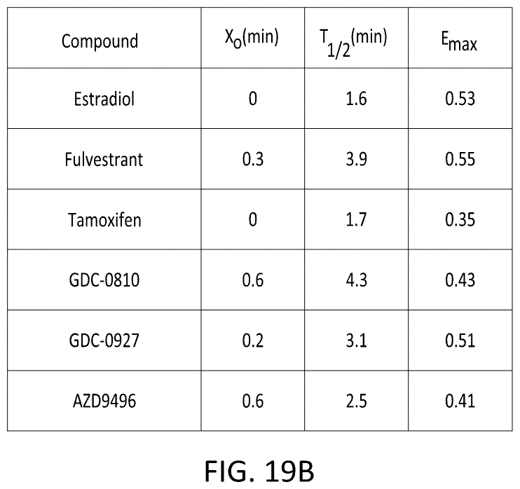

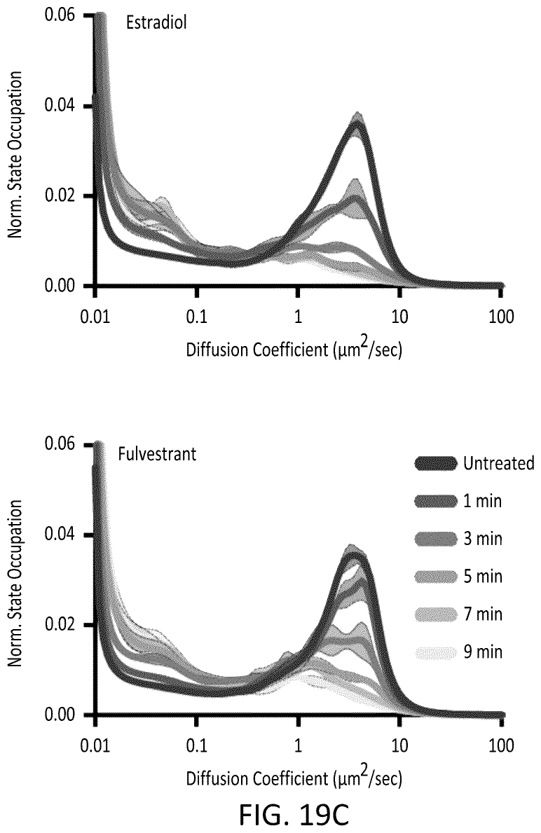

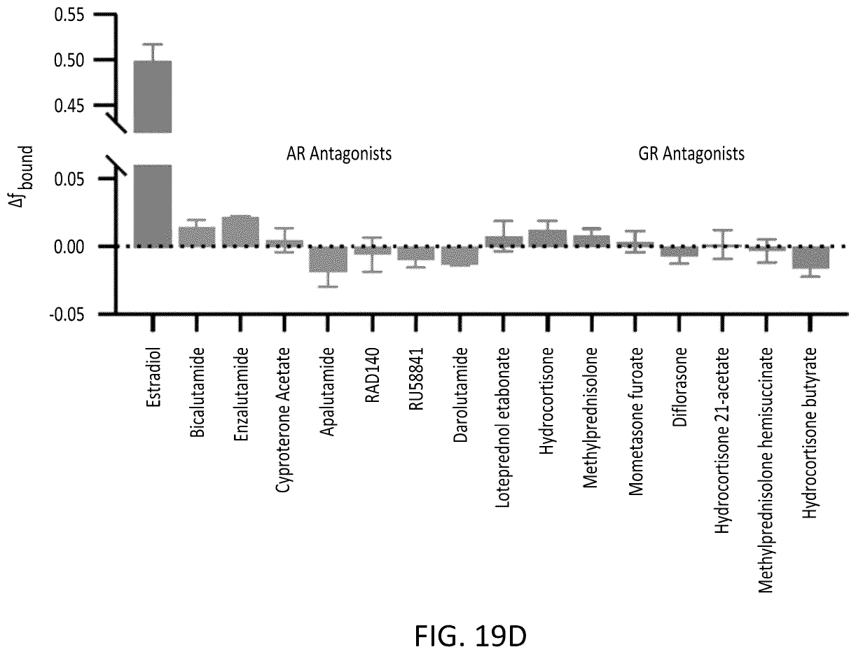

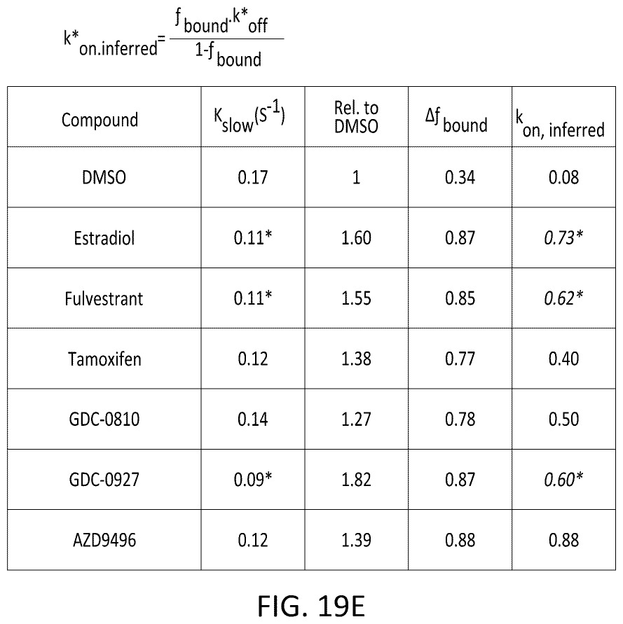

For example, but not by way limitation, a 5,067-molecule bioactive screen surprisingly revealed that all the known ER modulators—both agonists like estradiol and potent antagonists like fulvestrant—caused an increase in f_{bound}. A subset of selective ER modulators (SERMs) and selective ER degraders (SERDs) were subsequently assessed in more detail. These molecules all bind competitively to the ER ligand binding domain. As in the bioactive screen, both SERDs and SERMS increased f_{bound} (FIG. 14A) and slightly decreased measured D_{free} (FIG. 19A), with potencies ranging from 9 pM for GDC-0927 to 4.8 nM for GDC-0810 (FIG. 14B). Despite different physical-chemical properties, all five increased f_{bound} within minutes of compound addition (FIG. 14C, FIG. 19B), with no evidence of transient diffusive states (FIG. 19C). Without being bound by theory, it appears that ER dissociation from the chaperone complex, dimerization, and chromatin binding occur on rapid and seemingly comparable timescales. Since individual steps in these transitions cannot be distinguished, the on-rate of the entire process was considered to have the effective rate constant k*ₒₙ. Importantly, selective antagonists of AR and GR did not induce significant modulation of ER movement, further highlighting the utility of the htSMT techniques described herein in characterizing the specificity of interactions between modulators of protein function and their cognate targets (FIG. 19D).

Interestingly, SERMs 4-hydroxytamoxifen (4OHT) and GDC-0810 show lower maximal increases in f_{bound} compared with the SERDs fulvestrant and GDC-0927 (FIG. 14D). Similar effects have been described previously using fluorescence recovery after photobleaching (FRAP), which was confirmed using the Halo-ER cell line (FIG. 14E). The delay in ER signal recovery after two minutes in FRAP was consistent with the changes in f_{bound} measured by SMT (FIG. 14F). Although FRAP was used to measure these f_{bound} differences, the technique suffers from challenges in scalability and depends heavily on prior assumption of the underlying movement in the sample. In contrast, the htSMT techniques described herein permit detailed characterization of the potency of 4OHT and GDC-0810 relative to other ER ligands in their ability to increase ER chromatin binding.

Neither FRAP nor htSMT can discriminate between recovery driven by an increase in residence time (decreasing k*_{off}) or increasing the rate of chromatin binding (increasing k*ₒₙ), either of which would result in increasing f_{bound}. By changing SMT acquisition conditions to reduce the illumination intensity and collect long frame exposures, only immobile proteins form spots. Under these imaging conditions, the distribution of track lengths provides a measure of relative residence times. Both agonist and antagonist treatment led to longer binding times compared to DMSO, as an indication that ligand binding decreases k*_{off} (FIG. 14G, FIG. 19E). Consistent with FRAP, estradiol, GDC-0927, and fulvestrant show longer binding times compared with other ER modulators. Using f_{bound} and k*_{off} measurements, one can infer the k*ₒₙ. In all cases the changes in dissociation rate are not proportional to the increase in f_{bound}, and so ligand-imposed increases in k*ₒₙ likely contribute to the observed change in the chromatin-associated ER fraction (FIG. 19E). Without being bound by theory, these data are consistent with a model wherein ER rapidly binds to chromatin irrespective of which molecule occupies the ligand binding domain, but some ligands induce a conformation that can be further stabilized on chromatin by cofactors. Consequently, these data are supportive of ER engaging chromatin in mechanistically different ways. An efficacious ER inhibitor may promote rapid and transient chromatin binding that fails to effectively recruit necessary cofactors to drive transcription.

### 4.3 KineticSMT

In certain implementations of the htSMT workflows described herein, the systems and methods are adapted to identify the rate at which changes in protein movement emerge. For example, in certain of such implementations, htSMT can be used to distinguish direct versus indirect effects. Additionally, or alternatively, identifying the rate at which changes in protein movement emerge provides the capability to assess cell permeability and/or active transport, target efflux and/or influx, target engagement on rate and/or target engagement off rate among other parameters.

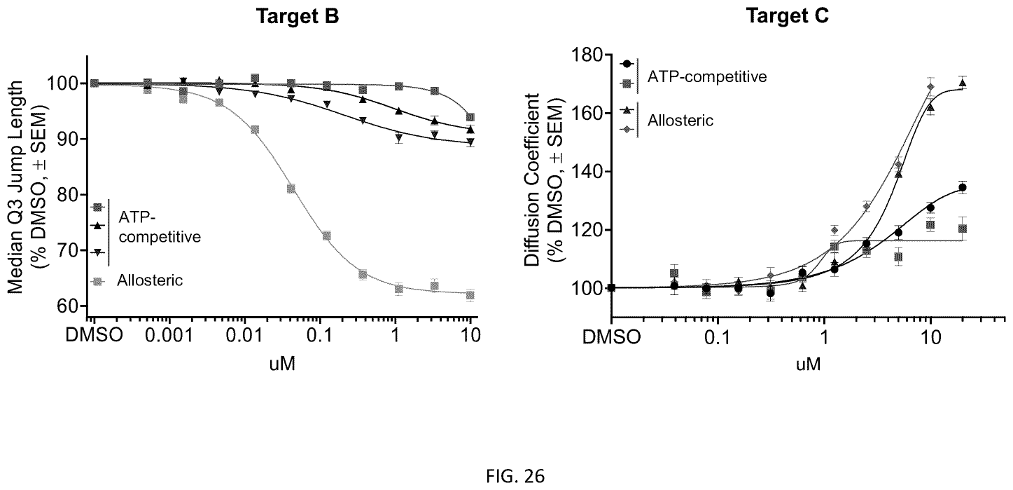

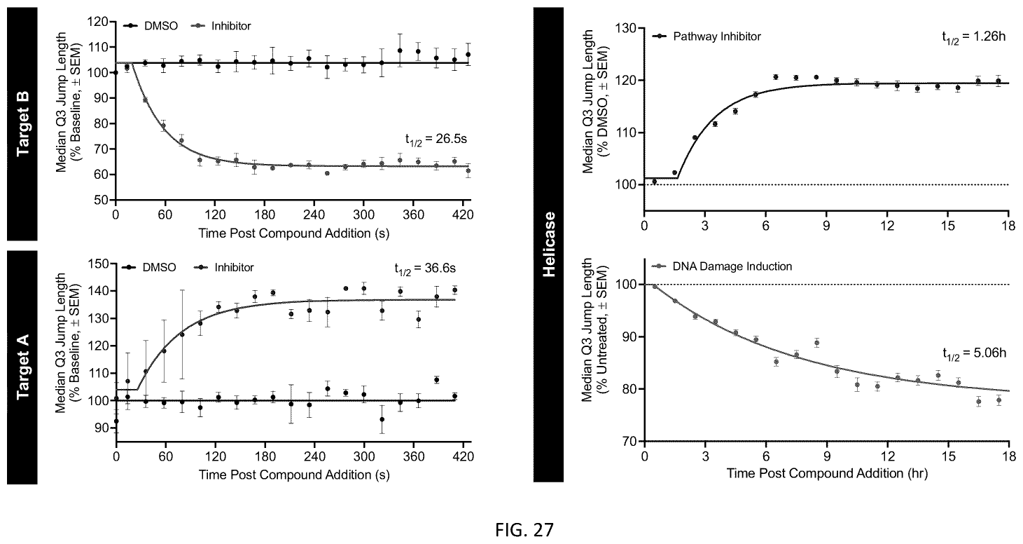

Given the live cell setting of SMT, a data collection mode was configured that allows for measurement of protein movement in set intervals after compound addition (kinetic SMT or kSMT). Both ER agonists and antagonists rapidly induce ER immobilization on chromatin when measured in kSMT (t_{1/2}=1.6 minutes for estradiol; FIG. 14C). On the other hand, HSP90 inhibitors like ganetespib and HSP990 exhibit a delay of 5 to 7 minutes before alterations in ER movement appear, after which an increase in f_{bound} with a t_{1/2} of 19.3 and 17.5 minutes was observed, respectively. The overall effect of these compounds reached a plateau after an hour (FIG. 16C). Proteasome inhibitors, e.g., bortezomib and carfilzomib, acted even more slowly, with changes in ER movement emerging only after 40 minutes, and slowly increasing over the four-hour measurement window (FIG. 16D). Similarly, differential kinetics of on-target, on-pathway, and off-target inhibitors that change movement in several additional targets have been measured, including Target A, Target B, and a helicase (FIG. 27). Hence this exploration of SMT kinetics represents an important tool that can facilitate differentiation between on-target and on-pathway modulators. The kSMT techniques described herein permit, for example, rapid mechanistic characterization of active compounds in a drug discovery setting.

To further differentiate the effect of pathway inhibitors on ER protein movement, relative ER residence times for each such molecule were characterized. Estradiol, SERMs, and SERDs all increased residence times and thus likely also increased the rate of ER association with chromatin (FIGS. 14A-14D, FIG. 19E). By contrast, while HSP90inhibition by HSP990 and ganetespib resulted in an increase in f_{bound}, a decrease was observed in the total number long binding events by two- and four-fold, respectively, while the binding times were similar to that observed with DMSO alone (FIG. 16E). These results indicate that HSP90 inhibition primarily increases k*ₒₙ while leaving k*_{off} largely unaffected. On the other hand, inhibition of the proteasome led to an increase in both the number and duration of long binding events. These results demonstrate that ER-chromatin binding can be modulated by changing the rate of association or disassociation, and that the inhibition of specific cellular partners can affect these rates differentially. Taken together with the different kinetics for direct ER, HSP90, and proteasome modulators, the instant data indicates that each class of molecule alters ER movement through separate mechanisms.

### 5. EXEMPLARY EMBODIMENTS

A. The present disclosure provides a method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
   (i) illuminating a field of view in a sample plane disposed within the sample with a light beam to cause fluorescence by a subset of the target fluorescent proteins in the live cells; and
   (ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and
(c) determining a change in the movement of the target fluorescent protein in the presence of the compound,
wherein the rate at which changes in the movement of the target fluorescent protein emerge is determined by comparing the changes in movement of the target fluorescent protein at the plurality of time points.

B. The present disclosure provides a method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
   (i) illuminating a field of view in a sample plane disposed within the sample with a light beam to cause fluorescence by a subset of the target fluorescent proteins in the live cells, wherein the subset of the target fluorescent produces 100-100,000 molecular trajectories in a single detected field of view; and
   (ii) detecting the fluorescence from a plurality of the target fluorescent proteins in the detected field of view of the sample plane via a detector device, wherein the detected field of view has a size of about 50 μm to less than 100 μm in a first dimension by about 50 μm to less than 100 μm in a second dimension; and
(c) determining a change in the movement of the target fluorescent protein in the presence of the compound,
wherein the rate at which changes in the movement of the target fluorescent protein emerge is determined by comparing the changes in movement of the target fluorescent protein at the plurality of time points.

C. The present disclosure provides a method determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
   (i) illuminating a field of view in a sample plane disposed within the sample with a light beam to cause fluorescence by a subset of the target fluorescent proteins in the live cells; and
   (ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and
(c) determining a change in the movement of target fluorescent proteins upon the addition of the compound, wherein the average change in movement of the target fluorescent proteins in the presence of the compound is at least 1%, at least 5%, at least 10% relative to the change observed in the absence of the compound,
wherein the rate at which changes in the movement of the target fluorescent protein emerge is determined by comparing the changes in movement of the target fluorescent protein at the plurality of time points.

D. The present disclosure provides a method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
   (i) illuminating a field of view in a sample plane disposed within the sample with a light beam to cause fluorescence by a subset of the target fluorescent proteins in the live cells;
   (ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and
   (iii) detecting the fluorescence from a plurality of the target fluorescent proteins in the detected field of view of the sample plane at a rate of >100 detected FOVs per day, >10,000 FOVs per day, or >100,000 detected FOVs per day; and
(c) determining a change in the movement of target fluorescent proteins in the presence of the compound,
wherein the rate at which changes in the movement of the target fluorescent protein emerge is determined by comparing the changes in movement of the target fluorescent protein at the plurality of time points.

E. The present disclosure provides a method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
   (i) illuminating a field of view in a sample plane disposed within the sample with a light beam to cause fluorescence by a subset of the target fluorescent proteins in the live cells; and
   (ii) detecting the fluorescence from a plurality of the target fluorescent proteins in the detected field of view of the sample plane via a detector device, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; and
(c) determining a change in the movement of the target fluorescent protein in the presence of the compound,
wherein the rate at which changes in the movement of the target fluorescent protein emerge is determined by comparing the changes in movement of the target fluorescent protein at the plurality of time points.

E1. The method of any one of A-E, wherein the change in movement detected is an increase in immobile trajectories indicating an increase in bound (f_{bound}) target fluorescent protein.

E2. The method of any one of A-E, wherein the change in movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(j) state occupation via inference.

E3. The method of any one of A-E2, wherein the target fluorescent protein interacts in a larger molecular assembly.

E4. The method of E3, wherein the target fluorescent protein is a ligand.

E5. The method of E3, wherein the target fluorescent protein is a receptor.

E6. The method of any one of A-E5, wherein the biological interaction is a direct interaction.

E7. The method of E6, wherein the direct interaction comprises binding of the compound to the target fluorescent protein.

E8. The method of any one of A-E5, where the biological interaction is an indirect interaction.

E9. The method of E8, wherein the indirect interaction comprises the compound agonizing or antagonizing a larger molecular assembly comprising the target fluorescent protein.

F. The present disclosure provides a method of determining a dose of a compound, wherein said compound induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
   (i) illuminating a field of view in a sample plane disposed within the sample with a light beam to cause fluorescence by at least a subset of the target fluorescent proteins in the live cells;
   (ii) detecting the fluorescence from one or more of the target fluorescent proteins in the sample plane via a detector device, wherein the detected field of view has a size of about 50 μm to less than 100 μm in a first dimension by about 50 μm to less than 100 μm in a second dimension; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

G. The present disclosure provides a method of determining a dose of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
   (i) illuminating a field of view in a sample plane disposed within the sample with a light beam to cause fluorescence by at least a subset of the target fluorescent proteins in the live cells, wherein the subset of the target fluorescent proteins produces 100-100,000 molecular trajectories in a single detected field of view;
   (ii) detecting the fluorescence from one or more of the target fluorescent proteins in the detected field of view of the sample plane via a detector device, wherein the detected field of view has a size of about 50 μm to less than 100 μm in a first dimension by about 50 μm to less than 100 μm in a second dimension; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

H. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
   (i) illuminating a field of view in a sample plane disposed within the sample with a light beam to cause fluorescence by at least a subset of the target fluorescent proteins in the live cells;
   (ii) detecting the fluorescence from one or more of the target fluorescent proteins in a detected field of view of the sample plane via a detector device, wherein the detected field of view has a size of about 50 μm to less than 100 μm in a first dimension by about 50 μm to less than 100 μm in a second dimension; and
(c) determining the rate at which changes in the movement of the target fluorescent protein occur in the presence of the compound, wherein the average change in movement of the target fluorescent protein in the presence of the compound is at least 1%, at least 5%, at least 10%, relative to the change observed in the absence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

I. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
   (i) illuminating a field of view in a sample plane disposed within the sample with a light beam to cause fluorescence by at least a subset of the target fluorescent proteins in the cells;
   (ii) detecting the fluorescence from one or more of the target fluorescent proteins in a detected field of view of the sample plane via a detector device, wherein the detected field of view has a size of about 50 μm to less than 100 μm in a first dimension by about 50 μm to less than 100 μm in a second dimension; and
   (iii) wherein said tracking comprises detecting the fluorescence from a plurality of the target fluorescent proteins in the detected field of view of the sample plane at a rate of >100 detected FOVs per day, >10,000 detected FOVs per day, or >100,000 detected FOVs per day; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

J. The present disclosure provides a method of determining a dose of a compound, wherein said compound induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
   (i) illuminating a field of view in a sample plane disposed within the sample with a light beam to cause fluorescence by at least a subset of the target fluorescent proteins in the live cells;
   (ii) detecting the fluorescence from one or more of the target fluorescent proteins in the sample plane via a detector device, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

J1. The method of any one of F-J, wherein the change in movement detected is an increase in immobile trajectories indicating an increase in bound (f_{bound}) target fluorescent protein.

J2. The method of any one of F-J, wherein the change in movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(j) state occupation via inference.

J3. The method of any one of F-J2, wherein the target fluorescent protein interacts in a larger molecular assembly.

J4. The method of J3, wherein the target fluorescent protein is a ligand.

J5. The method of J3, wherein the target fluorescent protein is a receptor.

J6. The method of any one of F-J5, wherein the biological interaction is a direct interaction.

J7. The method of J6, wherein the direct interaction comprises binding of the compound to the target fluorescent protein.

J8. The method of any one of F-J5, where the biological interaction is an indirect interaction.

J9. The method of J8, wherein the indirect interaction comprises the compound agonizing or antagonizing a larger molecular assembly comprising the target fluorescent protein.

K. The present disclosure provides a microscopy system configured to detect the rate of emergence of a biological interaction between a compound and a target fluorescent protein in a live cell comprising:
(a) a stage for supporting a sample, wherein the sample comprises a population of live cells, and where the live cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane and wherein the detected field of view has a size of about 50 μm to less than 100 μm in a first dimension by about 50 μm to less than 100 μm in a second dimension;
(e) a detector device for monitoring the light-based response from the target fluorescent proteins in the presence of the compound at a plurality of time points;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

L. The present disclosure provides a microscopy system configured to detect the rate of emergence of a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view in the sample plane, wherein the subset of the target fluorescent proteins produces 100-100,000 molecular trajectories in a single detected field of view and wherein the detected field of view has a size of about 50 μm to less than 100 μm in a first dimension by about 50 μm to less than 100 μm in a second dimension;
(d) a detector device for monitoring the light-based response from the target fluorescent proteins in the presence of the compound at a plurality of time points;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

M. The present disclosure provides a microscopy system configured to detect the rate of emergence of a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane and wherein the detected field of view has a size of about 50 μm to less than 100 μm in a first dimension by about 50 μm to less than 100 μm in a second dimension;
(d) a detector device for monitoring the light-based response from the target fluorescent proteins in the presence of the compound at a plurality of time points, wherein the average change in movement of the target fluorescent protein in the presence of the of the compound is at least 2%, at least 5%, at least 10%, relative to the change observed in the absence of the compound,
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

N. The present disclosure provides a microscopy system configured to detect the rate of emergence of a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a plurality of the target fluorescent proteins in the sample are disposed in a detected field of view of the sample plane and wherein the detected field of view has a size of about 50 μm to less than 100 μm in a first dimension by about 50 μm to less than 100 μm in a second dimension;
(d) a detector device for monitoring the light-based response from the target fluorescent proteins in the presence of the compound at a plurality of time points, wherein said monitoring comprises detecting the fluorescence from a plurality of the target fluorescent proteins in the detected field of view of the sample plane at a rate of >100 detected FOVs per day, >10,000 detected FOVs per day, >100,000 detected FOVs per day;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

O. A microscopy system configured to detect the rate of emergence of a biological interaction between a compound and a target fluorescent protein in a live cell comprising:
(a) a stage for supporting a sample, wherein the sample comprises a population of live cells, and where the live cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane, wherein the detected field of view has a size of about 50 μm to less than 100 μm in a first dimension by about 50 μm to less than 100 μm and wherein up to 70% of the detected field of view achieves sufficient laser illumination for tracking protein movement;
(e) a detector device for monitoring the light-based response from the target fluorescent proteins in the presence of the compound at a plurality of time points;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

O1. The system of any one of K-O, wherein the change in movement detected is an increase in immobile trajectories indicating an increase in bound (f_{bound}) target fluorescent protein.

O2. The system of any one of K-O, wherein the movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(j) state occupation via inference.

O3. The system of any one of K-O2, wherein the target fluorescent protein interacts in a larger molecular assembly.

O4. The system of O3, wherein the target fluorescent protein is a ligand.

O5. The system of O3, wherein the target fluorescent protein is a receptor.

O6. The system of any one of K-O5, wherein the biological interaction is a direct interaction.

O7. The system of O6, wherein the direct interaction comprises binding of the compound to the target fluorescent protein.

O8. The system of any one of K-O5, wherein the biological interaction is an indirect interaction.

O9. The system of claim O8, wherein the indirect interaction comprises the compound agonizing or antagonizing a larger molecular assembly comprising the target fluorescent protein.

### 6. EXAMPLES

### Example 1: High Throughput Single Molecule Tracking of Steroid Hormone Receptors

### A. Introduction

Steroid hormone receptors (SHRs) are a class of transcription factors that play crucial roles in normal human development and in disease pathogenesis. SHRs like the estrogen receptor (genes ESR1 and ESR2), androgen receptor (AR) and progesterone receptor (PR), as examples, contribute decisively to the acquisition of secondary sex characteristics, while the glucocorticoid receptor (GR) helps to orchestrate both metabolism and inflammation. In their ligand-free state, SHRs are kept sequestered in multiprotein complexes by the chaperone HSP9021. Canonically, in the presence of hormone they dimerize and bind their cognate genomic response elements, recruiting epigenetic modifiers and transcription machinery. At the same time, steroid hormone receptor-derived signals impose a large disease burden by promoting the growth of breast cancers (ER) or prostate cancers (AR) or by imposing immune and metabolic dysfunction (GR). SHRs therefore provide an excellent proof-of-concept system for the study of protein movement as a determinant of protein function due to the wealth of information and reagents already available for these systems, as well as previous reports characterizing some aspects of their cellular movement.

This example describes industrial scale htSMT techniques, systems incorporating such htSMT techniques, hardware and software related to such htSMT techniques, as well as methods of using such htSMT techniques. For example, the htSMT techniques described herein are capable of measuring protein movement in >1,000,000 cells per day. In addition, using ER as a proof-of-concept system, the htSMT techniques described herein exhibit specific, robust, and reproducible results. The htSMT techniques described herein can be used for a variety of applications including, but not limited to, classical drug discovery activities, such as compound library screening and the elucidation of SAR. Importantly, the htSMT techniques described herein can be used to characterize both known and novel pathway contributions to interaction networks, such as protein signaling interaction networks.

### B. Results

### a. Creation and Validation of an htSMT System

A robotic system capable of handling reagents, collecting high-quality, fast SMT image series, processing time-ordered raw images to yield molecular trajectories, and extracting features of biological interest within defined cellular compartments was developed (FIG. 1). To examine htSMT system performance across a broad spectrum of diffusion coefficients, three U2OS cell lines expressing HaloTag fused proteins with well-established behaviors in the cell were generated. Histone H2B-Halo, which is predominantly incorporated into chromatin and therefore effectively immobile over short timescales, was employed to estimate localization error. A prenylation motif (Halo-CaaX) embedded in the plasma membrane exhibits moderate diffusion. Unfused HaloTag was chosen to represent the upper limit of cellular “free” diffusion. Single-molecule trajectories measured in these cell lines yielded the expected diffusion coefficients (FIG. 11A). Using the immobile H2B-Halo trajectories, the localization error of the htSMT system was found to be 39 nm (FIG. 17A), comparable to other benchmark stroboscopic illumination datasets.

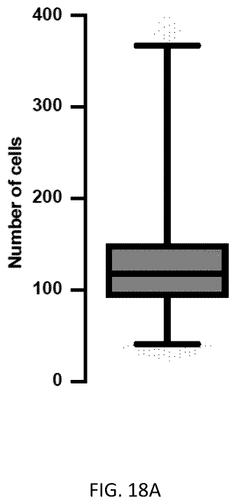

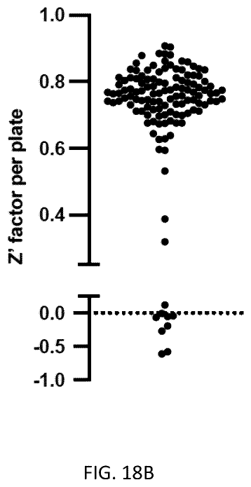

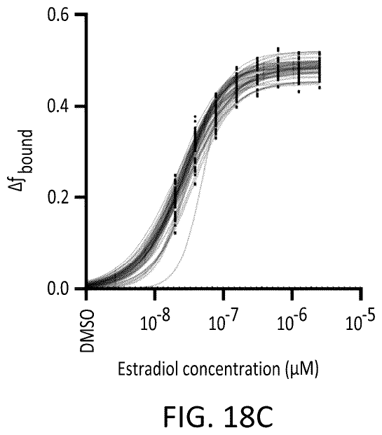

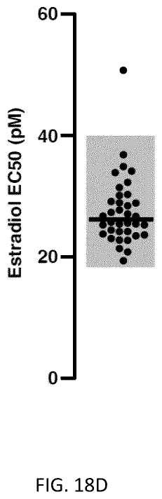

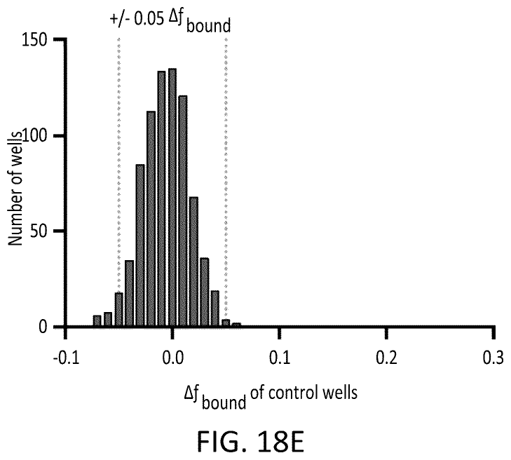

Whether the htSMT platform can extract accurate molecular trajectories at scale was tested. 384-well plates were employed where free Halo, Halo-CaaX, and H2B-Halo cell lines were mixed in equal proportions in each well. Imaging with a 94 μm by 94 μm field-of-view (FOV) achieved an average of 10 nuclei simultaneously (FIG. 11B, FIG. 18B), enough that most FOVs contained cells from each cell line. To limit ambiguity in cell assignment, only the tracks that fell within nuclear-segmented regions were considered. The probability distribution of diffusion states cleanly distinguishes between the three cell types (FIG. 11C). More importantly, by looking at the single-cell state distribution profiles of 103,757 cells from five separate 384-well plates, grouped by their distribution profile, highly consistent estimates of protein movement at the single cell level were recovered (FIG. 11D).

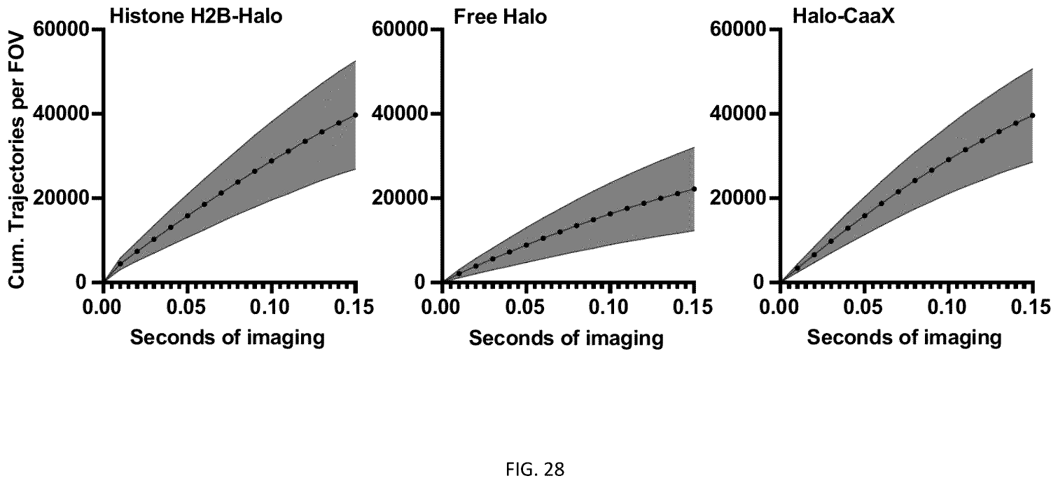

While single-cell measurements are powerful, the number of trajectories in one cell are limited, and so estimates of diffusive states can be broad. Combining trajectories from multiple cells, however, provides the expected distribution of diffusive states (FIG. 11E). Moreover, combining trajectories derived from many cells makes SpotOn or State Array analysis possible, where as few as 10³ trajectories permit satisfactory inference of the underlying diffusion states. Only a few seconds of imaging per FOV yield enough trajectories (>10,000) to accurately estimate protein movement, bringing the overall throughput of the platform to 13,000 individual wells (>90,000 FOVs per day; >1,000,000 cells/day), a rate of data acquisition that enables drug screening on a feasible timescale (FIG. 28).

### b. Using htSMT to Measure Protein Movement of SHRs

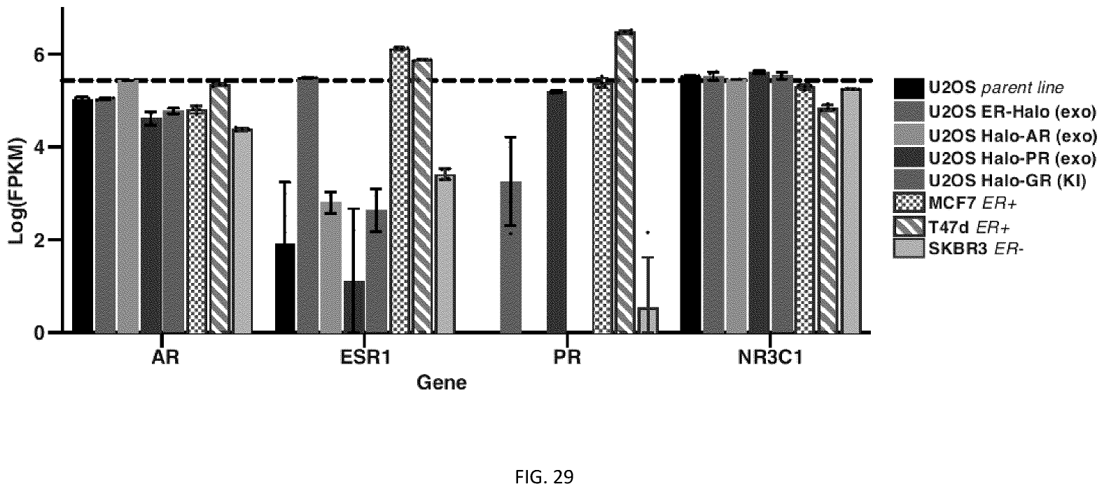

Equipped with an htSMT system capable of measuring protein movement broadly, the following work establishes that measurements of protein movement can be used to characterize protein activity. SHRs transition between inactive and active states via ligand binding (FIG. 12A), and htSMT can capture these differences. To that end, HaloTag fusion ER, AR, PR, and GR cell lines in a U2OS cell background were prepared to minimize effects of comparing movement in different cell types. Clones were carefully selected such that the HaloTag fusion SHRs were comparable to each other in transcript abundance, and not higher than transcript levels in tissue-specific cell lines like MCF7 and T47d, which are both ER and PR positive (FIG. 29).

In the absence of hormone, all four proteins exhibit similar movement profiles: a small immobile fraction and a large freely diffusing fraction with a 3.4-4.3 μm²/sec average diffusion coefficient (FIG. 12B). No correlation between diffusion and protein size was observed, highlighting the differences between cellular protein movement versus purified systems. Upon addition of agonist, a dramatic increase in immobile trajectories is observed, which is attributable to chromatin binding. The bound fraction (f_{bound}) is defined for each SHR as the fraction of tracks diffusing less than 0.1 μm²/sec (FIG. 12B). Consistent with previous findings, some SHRs had a higher proportion of bound molecules than others, both with and without ligand. The ligand-induced effect is most pronounced for ER, with 34% bound in basal conditions and 87% bound after estradiol treatment (FIG. 12B).

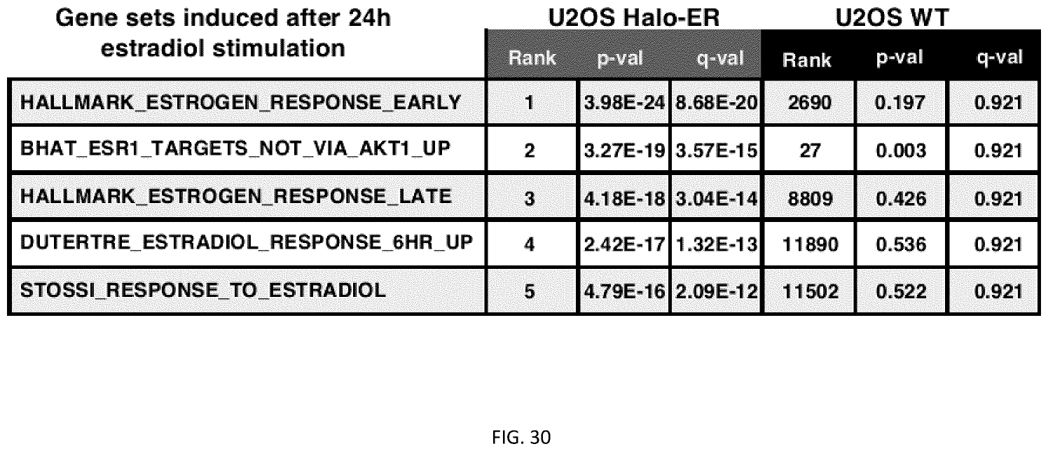

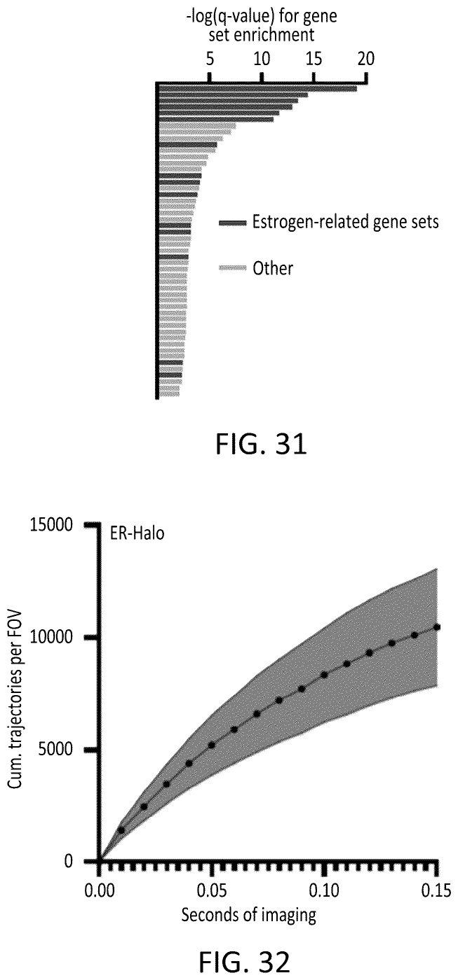

SHRs are highly selective for their cognate agonists in biochemical binding assays, which was confirmed by measuring the dose-dependent change in movement as a function of agonist concentration. The maximal increase in f_{bound} (FIG. 12C) and decrease in free diffusion coefficient (D_{free}; FIG. 12D) differed between SHRs. The dose titration curves also showed variable potencies (EC50) for each SHR/hormone pair, with ER-estradiol being both the most potent and most selective pair. RNA-seq after estradiol stimulation showed a marked induction of hallmark ER-dependent gene sets, confirming that the increase in chromatin binding observed by SMT has a functional effect in promoting ER-responsive gene programs, even in the ectopic expression setting (FIG. 30 and FIG. 31). Thus, SMT can precisely and accurately differentiate the ligand/target specificity directly within the living cellular environment.

### c. Screening a Diverse Bioactive Chemical Set Identifies Known and Novel Modulators of ER Movement

Characterization efforts of ligand selectivity for AR, ER, GR and PR collectively suggested that SMT can be used to interrogate the effects of compounds on protein dynamics at a throughput conducive to high throughput screening. The specificity and sensitivity of the htSMT platform was examined next. A structurally diverse set of 5,067 molecules with heterogeneous biological activities against ER was screened, assessing change in f_{bound} at 1 μM compound versus DMSO (FIG. 13). The screen was run twice to assess reproducibility, showing a high degree of agreement between replicates for ER-active molecules (FIG. 13). Each compound measurement was averaged from SMT trajectories of between 94 and 161 cells (25^{th} to 75^{th} percentiles; FIG. 18A). This screen illustrates the important advantages of the htSMT platforms described herein over more manual lower-throughput approaches.

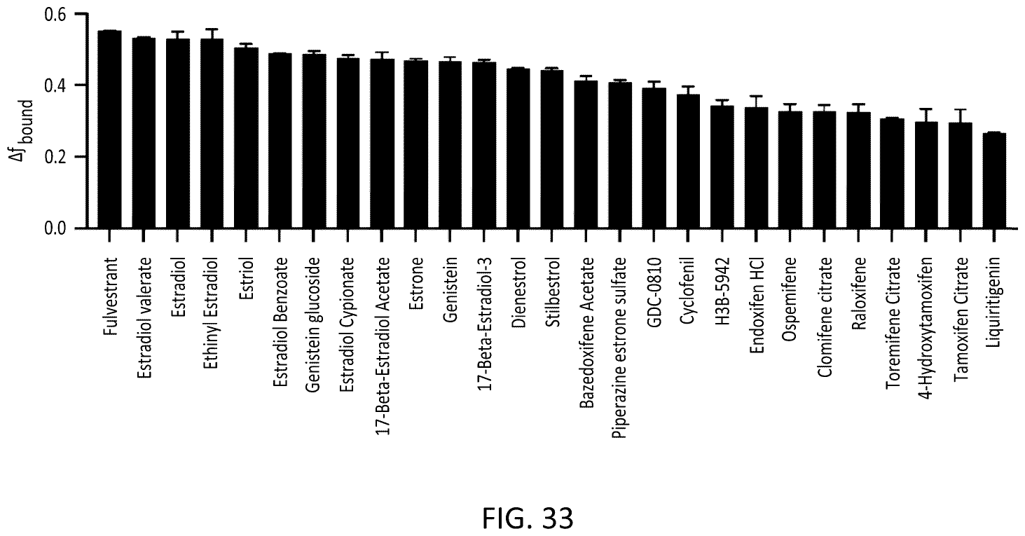

From plate to plate, the assay window for the screen was robust (FIG. 18B; average Z′-factor=0.79) and the measured potency of the control estradiol in each instance remained within three-fold of the mean (FIGS. 18C and 18D) and the distribution of negative control wells centered tightly on zero (FIG. 18E). Of the 30 compounds identified from the bioactive set expected to modulate ER, either as agonists or antagonists, all significantly increased f_{bound} measured by SMT, including notable examples such as 4-hydroxytamoxifen, fulvestrant, and bazedoxifene (FIG. 13 and FIG. 33).

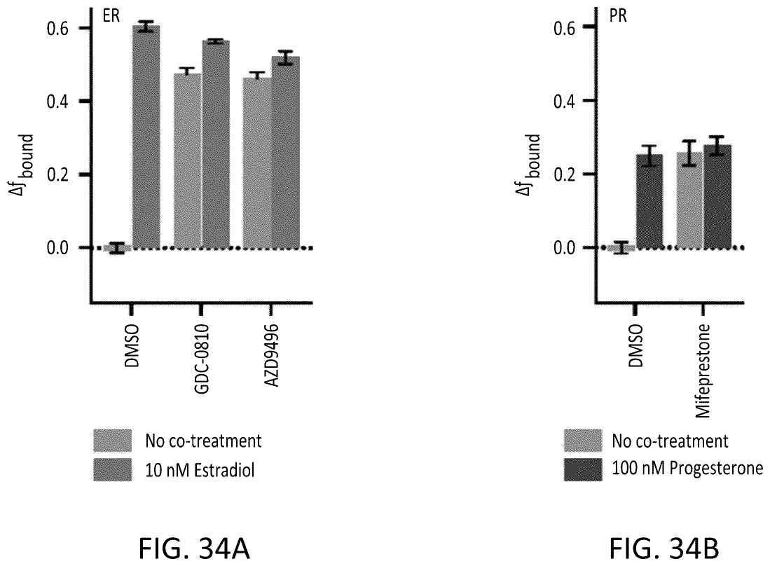

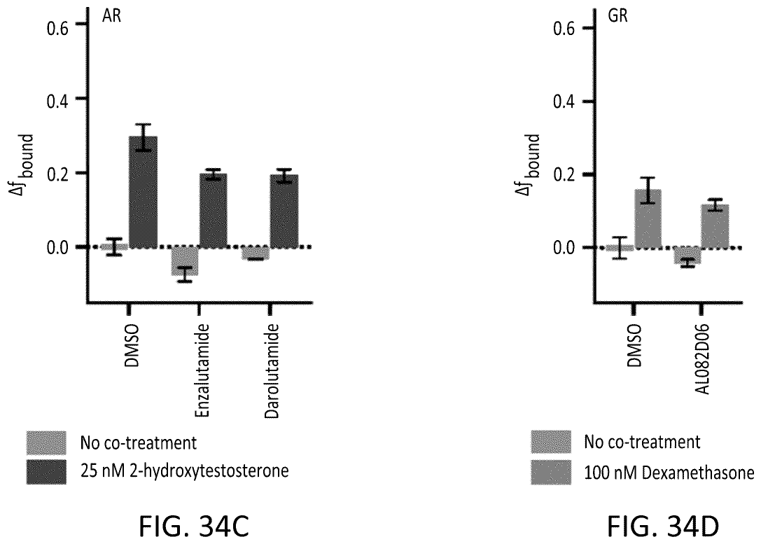

The somewhat counter-intuitive finding that both strong agonism or antagonism can lead to an increase in chromatin binding has been reported for ER, but this appears not to be a general feature of SHRs. While the PR antagonist mifepristone behaves similarly to ER antagonists (FIGS. 34A-34B), antagonists of AR like Enzalutamide and Darolutamide, and antagonists of GR like AL082D06 cause a decrease in chromatin binding. This decrease occurs when administered singly or when co-administered in competition with the cognate agonist (FIGS. 34C-34D). These results show how the cellular context and interaction partners are critical to understand the effect of a compound on its intended target. To underscore this point, in addition to binders of the ER ligand-binding domain, a number of active compounds targeting diverse nodes in the ER interaction network, including modulators of the proteasome, chaperones, kinases, and others were identified (FIG. 16A).

### d. Cellular ER Movement Elucidate Structure-Activity Relationships (SAR) of ER Modulators

The 5,067-molecule bioactive screen revealed that, surprisingly, all the known ER modulators—both agonists like estradiol and potent antagonists like fulvestrant—caused an increase in f_{bound}. A subset of selective ER modulators (SERMs) and selective ER degraders (SERDs) were subsequently assessed in more detail. These molecules all bind competitively to the ER ligand binding domain. As in the bioactive screen, both SERDs and SERMS increased f_{bound} (FIG. 14A) and slightly decreased measured D_{free} (FIG. 19A), with potencies ranging from 9 pM for GDC-0927 to 4.8 nM for GDC-0810 (FIG. 14B). Despite different physical-chemical properties, all five increased f_{bound} within minutes of compound addition (FIG. 14C, FIG. 19B), with no evidence of transient diffusive states (FIG. 19C). Without being bound by theory, it appears that ER dissociation from the chaperone complex, dimerization, and chromatin binding occur on rapid and seemingly comparable timescales. Since individual steps in these transitions cannot be distinguished, the on-rate of the entire process was considered to have the effective rate constant k*ₒₙ. Importantly, selective antagonists of AR and GR did not induce significant modulation of ER movement, further highlighting the utility of the htSMT techniques described herein in characterizing the specificity of interactions between modulators of protein function and their cognate targets (FIG. 19D).

Interestingly, SERMs 4-hydroxytamoxifen (4OHT) and GDC-0810 show lower maximal increases in f_{bound} compared with the SERDs fulvestrant and GDC-0927 (FIG. 14D). Similar effects have been described previously using fluorescence recovery after photobleaching (FRAP), which was confirmed using the Halo-ER cell line (FIG. 14E). The delay in ER signal recovery after two minutes in FRAP was consistent with the changes in f_{bound} measured by SMT (FIG. 14F). Although FRAP was used to measure these f_{bound} differences, the technique suffers from challenges in scalability and depends heavily on prior assumption of the underlying movement in the sample. In contrast, the htSMT techniques described herein permit detailed characterization of the potency of 4OHT and GDC-0810 relative to other ER ligands in their ability to increase ER chromatin binding.

Neither FRAP nor htSMT can discriminate between recovery driven by an increase in residence time (decreasing k*_{off}) or increasing the rate of chromatin binding (increasing k*ₒₙ), either of which would result in increasing f_{bound}. By changing SMT acquisition conditions to reduce the illumination intensity and collect long frame exposures, only immobile proteins form spots. Under these imaging conditions, the distribution of track lengths provides a measure of relative residence times. Both agonist and antagonist treatment led to longer binding times compared to DMSO, as an indication that ligand binding decreases k*_{off} (FIG. 14G, FIG. 19E). Consistent with FRAP, estradiol, GDC-0927, and fulvestrant show longer binding times compared with other ER modulators. Using f_{bound} and k*_{off} measurements, one can infer the k*ₒₙ. In all cases the changes in dissociation rate are not proportional to the increase in f_{bound}, and so ligand-imposed increases in k*ₒₙ likely contribute to the observed change in the chromatin-associated ER fraction (FIG. 19E). Without being bound by theory, these data are consistent with a model wherein ER rapidly binds to chromatin irrespective of which molecule occupies the ligand binding domain, but some ligands induce a conformation that can be further stabilized on chromatin by cofactors. Consequently, these data are supportive of ER engaging chromatin in mechanistically different ways. An efficacious ER inhibitor may promote rapid and transient chromatin binding that fails to effectively recruit necessary cofactors to drive transcription.

### e. htSMT Can Define Relevant Structure Activity Relationships for ER Antagonists

As the name implies, next-generation ER degraders like GDC-0927, AZD9833, and GDC-9545 were optimized to enhance degradation of ER. Compound-induced ER degradation via immunofluorescence was indeed observed both in established breast cancer model lines and the U2OS ectopic expression system (FIG. 15A, FIG. 20A). Structural analogs of GDC-0927 have been reported and optimized for ER degradation, however the correlation between ER degradation and cell proliferation is poor (FIG. 15B, FIG. 20B-20D). By measuring protein movement, however, more precise measurements of inhibitory activity can be obtained than can be achieved by assessing protein degradation. The potency and maximal effect of structural analogues of GDC-0927 was determined using htSMT. Overall, these analogues exhibited a potency range of 15 pM to 12 nM and increased ER f_{bound} by 0.4 to 0.56 (FIG. 15C). Small changes in the chemical structure produced measurable changes in both compound potency and maximal efficacy as determined using SMT.

The potencies of GDC-0927 and analogues determined either via ER degradation or SMT were compared to the ability of each of these compounds to block estrogen-induced breast cancer cell proliferation. Potency assessed by ER degradation was not a good predictor of potency in the cell proliferation assay (FIG. 15C). By contrast, SMT measurements of f_{bound} strongly correlate with cell viability (FIG. 15E; R² of 0.83 for T47d and 0.84 for MCF7). Intriguingly, SMT EC₅₀ values were on average 10-fold lower than those observed in the cell growth assay, indicating that SMT is sensitive enough to permit the selection of chemical series which would not show effects in other cellular assays. This correlation between effects on protein movement (e.g., f_{bound}) and protein function (suppression of cell proliferation) coupled with the throughput of the SMT system make this an attractive approach for the identification of protein modulators with novel properties.

### f. Individual Pathway Interactors Show Unique Phenotypes Related to Their Effects on ER Motility

In addition to known ER active modulators, many other compounds in our bioactive library provoked easily measurable changes in f_{bound}. To define a threshold for calling a molecule from the screen “active”, 92 compounds with different magnitudes of change in f_{bound} were selected to retest in a dose titration (FIG. 21A, 21B). A 5% change in f_{bound} was sufficient to reproducibly distinguish active compounds. Using this approach, 239compounds were identified in the bioactive library that affected the ER mobility (FIG. 13). Among these compounds, the correlation between the two screen replicates was high (R²=0.92) and the level of activity was reproducible (the slope for active molecules was 0.94). Some active compounds could be clustered based on scaffold homology, but most clusters consisted of one or only a few members (FIG. 21C, 21D). Structural clustering was employed to identify known ER modulators where the vendor-provided annotation was poorly defined (FIG. 20C). These results demonstrate that htSMT is reproducible and robust when screening large collections of molecules.

Most active molecules from the screen were not structurally related to steroids (FIG. 21C, 21D). On the other hand, many compounds could be grouped based on their reported biological targets or pathways (FIG. 16A, FIG. 22A, 22B). For example, heat shock protein (HSP) and proteasome inhibitors consistently increased f_{bound}, whereas cyclin dependent kinase (CDK) and mTOR inhibitors decreased f_{bound}. Though many CDK inhibitors lack within-family specificity (FIG. 22A, pan-CDK), CDK9-specific inhibitors were found to more strongly affected ER movement than did CDK4/6-specific inhibitors. Furthermore, as with selective AR and GR antagonists, inhibitors targeting ALK, BTK, and FLT3 kinases that have not been shown to interact with ER have no impact on ER movement when assessed using SMT (FIG. 16A).

For the inhibitors of cellular pathways that were identified, a dose titration was used to better characterize the effect of each on ER movement. Potencies ranged from the sub-nanomolar to low micromolar (FIG. 16B), similar to the reported potencies of these compounds against their cellular targets. Additionally, these molecules were tested against AR (FIG. 22C) and PR (FIG. 22D). Each SHR differed meaningfully from the others in terms of the response to compounds identified through an ER-focused screening effort. Again, the magnitude of ER SMT effect was largely consistent within a target class (FIG. 16B, FIG. 22A-22D). The finding that structurally distinct compounds exhibited similar effects based on their biological targets favors the view that these biological targets must themselves interact with ER, and that the compounds therefore affect ER movement indirectly. HSP90 is a chaperone for many proteins, including SHRs. In the canonical model, hormone binding releases the SHR-HSP90 complex. Indeed, HSP90 inhibitors increased f_{bound} for ER, AR, and PR, consistent with one function of the chaperone being to adjust the equilibrium of SHR binding to chromatin (FIG. 22B-22D). Proteasome inhibition also leads to ER immobilization on chromatin, which aligns with the results obtained in the htSMT screen of bioactive compounds. ER has been shown to be phosphorylated by CDK, Src, or GSK-3 through MAPK and PI3K/AKT signaling pathways, and therefore inhibition of these pathways would reasonably be expected to affect ER movement measured using SMT. While CDK inhibition led to an increase in ER mobility, inhibition of PI3K, AKT, or other upstream kinases showed no effect (FIG. 16A).

Interestingly, SMT movement of an ER triple point mutant engineered to lack previously defined phosphorylation sites important for transactivation (S104A/S106A/S118A) were affected by CDK and mTOR pathway inhibitors (FIG. 23), which indicates that additional phosphorylation sites can mediate the effects of CDK9 and PI3K/AKT signaling, or that other molecular targets of CDK and PI3K/AKT can act indirectly to alter the movement of ER. The change in ER protein movement for characterized pathway inhibitors such as those targeting CDK and mTOR is subtle but consistent across compounds, indicating biological meaning in these observations and highlight the need for accurate and precise SMT measurements. Hence the htSMT techniques described herein offer avenues to provide comprehensive pathway interaction information.

Since SMT can identify compounds that act either directly on a target or through some intermediary process, strategies to distinguish between these alternative modes of action were pursued. For example, by investigating the rate at which changes in protein movement emerge, SMT can be used to distinguish direct versus indirect effects on ER activity. Given the live cell setting of SMT, a data collection mode was configured that allows for measurement of protein movement in set intervals after compound addition (kinetic SMT or kSMT). Both ER agonists and antagonists rapidly induce ER immobilization on chromatin when measured in kSMT (t_{1/2}=1.6 minutes for estradiol; FIG. 14C). On the other hand, HSP90 inhibitors like ganetespib and HSP990 exhibit a delay of 5 to 7 minutes before alterations in ER movement appear, after which an increase in f_{bound} with a t_{1/2} of 19.3 and 17.5 minutes was observed, respectively. The overall effect of these compounds reached a plateau after an hour (FIG. 16C). Proteasome inhibitors, e.g., bortezomib and carfilzomib, acted even more slowly, with changes in ER movement emerging only after 40 minutes, and slowly increasing over the four-hour measurement window (FIG. 16D). Hence this exploration of SMT kinetics represents an important tool that can facilitate differentiation between on-target and on-pathway modulators. The kSMT techniques described herein permit, for example, rapid mechanistic characterization of active compounds in a drug discovery setting.

To further differentiate the effect of pathway inhibitors on ER protein movement, relative ER residence times for each such molecule were characterized. Estradiol, SERMs, and SERDs all increased residence times and thus likely also increased the rate of ER association with chromatin (FIG. 14, FIG. 19E). By contrast, while HSP90 inhibition by HSP990 and ganetespib resulted in an increase in f_{bound}, a decrease was observed in the total number long binding events by two- and four-fold, respectively, while the binding times were similar to that observed with DMSO alone (FIG. 16E). These results indicate that HSP90 inhibition primarily increases k*ₒₙ while leaving k*_{off} largely unaffected. On the other hand, inhibition of the proteasome led to an increase in both the number and duration of long binding events. These results demonstrate that ER-chromatin binding can be modulated by changing the rate of association or disassociation, and that the inhibition of specific cellular partners can affect these rates differentially. Taken together with the different kinetics for direct ER, HSP90, and proteasome modulators, the instant data indicates that each class of molecule alters ER movement through separate mechanisms.

### C. Methods

### a. Cell Lines

U2OS (ATCC Cat. No. HTB-96), MCF7 (ATCC Cat. No. HTB-22), T47d (ATCC Cat. No. HTB-133) and SK-BR-3 (ATCC Cat. No. HTB-30) were grown in DMEM (Cat. No. 1056601, Gibco DMEM, high glucose, GlutaMAX Supplement, Thermofisher) supplemented with 10% Fetal Bovine Serum (Cat. No. 16000044, Thermofisher) and 1% pen-strep (Cat. No 15140122, Thermo Fisher) and maintained in a humidified 37° C. incubator at 5% CO₂ and subcultivated approximately every two to three days.

### b. HaloTag-Expressing Cell Lines

For ER, AR, and PR-HaloTag fusions, mammalian expression vectors containing the fusion gene under the control of a weak L30 promoter and containing a Neomycin resistance marker were transfected into U2OS cells at 70% confluence using FuGENE 6 (Cat. No. E2691, Promega). Transfected cells were selected with G418 (Cat. No. 10131027, Thermo Fisher) at 500 μg/mL, then clonally isolated. Clones expressing the desired fusion gene were determined first by staining with 100 nM JF₅₄₉-HTL (Cat. No. GA1110, Promega) and 50 nM Hoechst 33342 and identifying clones with the expected distribution of JF₅₄₉ signal. Between three and six clones were subsequently tested using SMT conditions for response to a control compound, and the most homogenous clones were subsequently expanded for further testing. Unless otherwise specified, all experiments are with a single, clonally isolated cell line. Because U2OS cells express GR endogenously, HaloTag was inserted right before the stop codon of endogenous NR3C1 via homology-directed repair using CRISPR/Cas9. The HaloTag knock-in was validated by imaging using HTL-JF₆₄₆ staining and through DNA sequencing.

### c. Western Blot

Cells were grown in the same conditions as described previously. 1.5×10⁶ cells were seeded per well in a 6-well plate in DMEM overnight, followed by compound treatment (DMSO or 100 nM fulvestrant) the following day for 24 hours. Cells are lysed in 200 μL 1× Cell Lysis Buffer (catalogue number 9803, Cell Signaling). Protein lysate concentration is then determined using BCA protein assay kit (Catalog number 23225, Pierce™ BCA Protein Assay Kit) following manufacturer instructions. Capillary Western Immunoassay were performed using Jess Protein Simple following manufacturer's instruction (protein simple, USA). Levels of αER (1:100, RM-9101) were normalized to loading control β-tubulin (1:100, NC0244815 LI-COR 92642213, Thermo Fisher). The peaks were analyzed with the Compass software (Protein Simple, USA).

### d. RNA-seq

Cells were seeded into 12-well tissue-culture treated plates at densities of 250,000cells (U2OS-WT), 200,000 cells (U2OS-ER), or 300,000 cells (MCF7, SK-BR-3, T47d) per well. 24 hours later, cells were treated with estradiol at a final concentration of 25nM for the indicated time-points (0 minutes, 10 minutes, 60 minutes, or 3 hours). To process cells for total RNA, cells were washed twice with ice-cold PBS, lysed with 350 uL Buffer RLT (Qiagen 79216), scraped off the plate (Fisher 08100241), frozen on dry ice and stored at −20 degrees C. Cell lysates were then thawed, homogenized using QIAshredder columns (Qiagen 79656), and processed through the Qiagen RNeasy Micro kit (Qiagen 74004) using the standard protocol and including the optional on-column DNase digestion step (Qiagen 79254). All samples had a RIN score of 10 by TapeStation (Agilent 5067-5576). RNA sequencing libraries were prepared from total RNA by Novogene (CA). In brief, mRNA was purified from total RNA using poly-T oligo-attached magnetic beads and fragmented. First-strand synthesis was performed using random hexamer primers, second-strand synthesis was performed using dTTP, and libraries were prepared after end repair, A-tailing, adapter ligation, amplification, and purification. Libraries were sequenced on an Illumina NovaSeq with paired 150 cycle reads. For data analysis, paired-end reads were aligned to the hg38 reference genome using Hisat2 v2.0.5, featureCounts v1.5.0-p3 was used to count the number of reads mapped to each gene, and differential expression analysis was performed using DESeq2 (1.20.0).

### e. Single Molecule Tracking Sample Preparation

Cells were seeded on tissue culture-treated 384-well glass-bottom plates at 6000 cells per well. Seeded cells were then incubated at 37° C. and 5% CO₂ to allow adhesion overnight. For all SMT experiments, cells were incubated with 5-100 pM of JF₅₄₉-HTL (Cat. No. GA1110, Promega) and 50 nM Hoechst 33342 for an hour in complete medium. Cells were then washed three times in DPBS and twice in imaging media, which is fluoroBrite DMEM media (Cat. No. A1896701, Thermo Fisher) supplemented with GlutaMAX (Cat. No. 35050079, Thermo Fisher) and the same serum and antibiotics as growth media. Where appropriate, compounds were serially diluted in an Echo Qualified 384-Well Low Dead Volume Source Microplate (0018544, Beckman Coulter) to generate dose-titration source material. Compounds were administered at a final 1:1000 dilution in cell culture medium. Each dose of a compound has at least 2 replicates per plate and 3 plate replicates, 20 DMSO control wells and 2 no dye control wells were randomized across each plate. Unless otherwise specified, compounds were allowed to incubate for an hour at 37° C. prior to image acquisition.

### f. Image Acquisition

Unless otherwise stated, all image acquisition using SMT was performed on a custom-built HILO microscope based on a Nikon Ti2, motorized stage, stage top environmental chamber (OKO labs), quad-band filter cube (Chroma), custom laser launch with 405 nm, and 561 nm wavelengths, delivering >10 mW and >150 mW of power to the back focal plane of the objective, respectively. Fluorescence emission was passed through a high-speed filter wheel (Finger Lakes Instruments) and collected with a backlit CMOS camera (Prime 95b, Teledyne). Images were acquired with a 60× 1.27 NA water immersion objective (Nikon). Environmental chamber was set to 37° Celsius, 95% humidity, and 5% CO₂. For each field of view, 200 SMT frames were collected at a frame rate of 100 Hz, with a 2 msec stroboscopic laser pulse. 10 frames of the Hoechst channel were collected at the same frame rate for downstream registration of tracks to nuclei.

### g. Image Analysis

Image acquisition produced one JF₅₄₉ movie and one Hoechst per field of view. The JF₅₄₉ movie was used to track the movement of individual JF₅₄₉ molecules, while the Hoechst movie was used for nuclear segmentation. Tracking was accomplished in three sequential steps—detection, subpixel localization, and linking—using a combination of existing methods. Briefly, spots were detected using a generalized log likelihood ratio detector. After detection, the estimated position of each emitter was refined to subpixel resolution using Levenberg-Marquardt fitting with an integrated 2D Gaussian spot model starting from an initial guess afforded by the radial symmetry method. Detected spots were linked into trajectories using a custom modification of a hill-climbing algorithm. The same detection, subpixel localization, and linking settings were used for all movies used in this manuscript.

For nuclear segmentation, all frames of the Hoechst movie were averaged to generate a mean projection. This mean projection was then segmented with a neural network trained on human-labeled nuclei. Each spot was assigned to at most one nucleus using its subpixel coordinates.

To recover movement information from trajectories, state arrays were used, a Bayesian inference approach, with the “RBME” likelihood function and a grid of 100 diffusion coefficients from 0.01 to 100.0 μm² s⁻¹ and 31 localization error magnitudes from 0.02 to 0.08 μm. After inference, localization error was marginalized out to yield a one-dimensional distribution over the diffusion coefficient for each field of view. For single-cell analysis, SMT and nuclear segmentation as performed on a mixture of U2OS cells bearing H2B-HaloTag, HaloTag-CaaX, or free HaloTag. The marginal likelihood of each of a set of 100 diffusion coefficients on the set of trajectories within each segmented nucleus was evaluated. These marginal likelihood functions were clustered with k-means (3 clusters), and the marginal likelihood functions for each cell were ordered by their cluster index to produce the heat map. To estimate the fraction bound (f_{bound}), the state array posterior distribution below 0.1 μm² s⁻¹ was integrated. To estimate the free diffusion coefficient (D_{free}), the mean of the posterior distribution above 0.1 μm² s¹ was computed.

### h. Data Analysis

Tracking results from the automated processing pipeline were analyzed using KNIME or Spotfire (TIBCO). Individual f_{bound} or D_{free} measurements were associated with experimental metadata and aggregated by condition. Change in f_{bound} was calculated as the difference between the f_{bound} of each well and the median f_{bound} of DMSO in the same plate. Wells that had no cells in the field of view or in which the field of view was out of focus were omitted from further analysis. Compounds were assessed for assay interference using the median fluorescence intensity of the tracking channel and omitted if it was more than 3 standard deviations higher than the median intensity of the DMSO wells. Similarly, plates where the active and negative controls could not be clearly resolved or where the significantly deviated from the performance of the rest of the screen were removed from further analysis. Finally, compound with a variance more than three standard deviations higher than the average compound variance (41 compounds; 0.08%) were removed from downstream analysis. Z′-factor between the active controls on a plate and DMSO was calculated as previously described. EC₅₀ values were calculated in Prism (GraphPad) by first log-transforming the molecule concentrations and then fitting to a four-parameter logistic curve

### i. Clustering Active Molecules

Chemical structure-based clustering was performed on molecules identified as active (239 in total). Molecular frameworks were computed as described by Murcko et al and as implemented in Pipeline Pilot. Molecular frameworks were clustered using functional class fingerprints (FCFP_4) with a similarity threshold cut-off of 0.3 Tanimoto distance. A total of 21 clusters were obtained with singletons being the major class (124 molecules). The next largest group was the flavone class represented by 27 members, followed by a couple of diverse classes within the steroidal class with 14 and 20 members respectively. The other category is the stilbene class with 7 members representing tamoxifen as one of the members. The remaining actives (47 molecules) were grouped into one 3-membered cluster and all the others with 2 members per cluster.

### j. Kinetic Experiments

Cells were seeded into a 384-well plate the day before, dyed, and washed as described above. 1 well with 25 FOVs per well were taken as a baseline reading. Then, while imaging, compound was manually added to each well to a final concentration of 100 nM. Data was then collected for 20 wells. A pause was included between each FOV such that the entire imaging regime covers the assay window. Change in f_{bound} was determined per-well relative to t=0.

For assays extending to 4 hours, the plate was imaged twice with 8 FOVs per well with different FOV locations per readthrough to prevent photobleaching from impacting data. All data presented represents was performed in three different biological replicates.

### k. Residence Time Imaging

Sample preparation and execution of residence time imaging experiments were conducted in a similar manner to the single molecule tracking assay described above with a few exceptions. Samples were dyed with 1-10 pM JF₅₄₉ (Promega) and 50 nM Hoechst 33342 for an hour. 400 frames per field of view were collected with a camera integration time was set to 250 msec, and laser sources reduced to 5 mW at the objective. During image acquisition, lasers were on continuously. Compound incubation ranged from 1 to 4 hours. At least 8 well replicates were collected per condition.

### l. Residence Time Analysis

Image processing, including spot detection, localization, and track reconnection were performed using the same methods described above. Because residence time imaging selectively tracks slow-diffusing molecules, individual localizations were limited to a 300 nm maximum displacement for individual jump reconnections. Sets of trajectories for each field of view were binned into 1-CDF distributions and fit to a two exponent decay model

$$CDF ( t ) = A ( {Fe}_{- {k}_{fast} t} + ( 1 - F ) {e}_{- {k}_{slow} t} ) CDFt = A ( Fe - kfastt + 1 - Fe - kslowt ) .$$

### m. Fluorescence Recovery After Photobleaching

Images were acquired on a custom-built HiLo microscope as described above with a Spectra Light Engine RS-232. Stimulation was directed using a miniscanner coupled with a Coherent OBIS 561 nm 100 mW laser. All imaging was performed using a 60× 1.27 NA water immersion objective (Nikon). All experiments were performed at 37° Celsius. For FRAP experiments, Cells were seeded into a 384-well plate the day before, labeled with 50 nM HTL-JF₅₄₉, and washed as described above. Compound was added to 100 nM final an hour before imaging. Then, a pre-bleach image was acquired by averaging 10 consecutive images. Then 8-10 regions were bleached (2 background, 6-8 cells) and 2 regions in cells were unbleached. Regions that were bleached were bleached at 10% power without scanning. For the next 30 seconds, an image was acquired every 200 ms, then every 1 second for 2 minutes. The background-subtracted average intensity was measured in the region of interest over time and normalized to the average of the fluorescence in the baseline images, then normalized to the unbleached regions to account for readout-induced photobleaching of fluorophores. Data from 18-24 cells were pooled per experiment for three biological experiments.

### n. Immunofluorescence

Cells were grown in conditions as described previously. Cells were seeded in glass bottom 384-well plates coated with 0.05 mg/ml PDL (Cat. No. A3890401, Thermofisher) at 6000 cells per well for Halo-ER U2OS cells and 8000 for MCF7 and T47d cells. Cells were grown overnight followed by compound treatment on the second day for 24 hours at 37° C. and 5% CO₂. Compounds were serially diluted in an Echo® Qualified 384-Well Low Dead Volume Source Microplate (0018544, Beckman Coulter) to generate a 21-point dose response at 1:3 dilution starting from a concentration of 10 mM. Compounds were administered at a final 1:1000 dilution in cell culture medium. An 8 to 12-point dose response was selected based on the potency of each compound. Each concentration was replicated at least once per plate and has at least 2 plate replicates. Cells were fixed by addition of paraformaldehyde (Cat. No. 15710-S; Electron Microscopy Sciences), with a final concentration of 4% for 20 minutes. Cells were then permeabilized using blocking buffer containing 1% bovine serum albumin and 0.3% Triton-X100 in 1× PBS for an hour at room temperature. Immunofluorescent staining of ER was carried out using αER antibody (1:500, RM-9101) diluted in the same blocking buffer for 1 hour at room temperature. Extensive washing with PBS was performed prior to secondary antibody staining. Secondary antibody staining was carried out using Alexa fluor 488 conjugate anti-rabbit IgG (1:1000, Cat. No. A32731, thermos Fisher) for an hour. Nuclear staining was carried out using Hoechst 33342 solution at 1 mg/ml. Imaging of immunofluorescence was done using the ImageXpress Micro (Molecular Devices) at 10× magnification and 4 field-of-view per well. Fluorescence intensity within the nucleus were quantified using CellProfiler. All analysis and curve fitting were carried out using Prism with DMSO as a baseline.

### o. Cell Proliferation

Cells were grown and seeded in conditions as described above. Cells were seeded in 384-well plates (Cat. No. 353963, Corning) at 1000 cells per well for Halo-ER U2OS, 1200 cells for SK-BR-3 and 1800 cells for MCF7 and T47d. Cells were grown overnight, then treated with compounds the following day. Compound concentration and administration are the same as described previously for the immunofluorescence assay. Plates are scanned in the IncuCyte live-cell analysis system (Sartorius) at 24-hour intervals for a total of 5 days using phase contrast. Cell proliferation quantification was carried out by the built-in analysis function using whole well confluency mask. All analysis and curve fitting were carried out using Prism with DMSO as a baseline.

### Example 2. htSMT Analysis of AR Agonists and Antagonists

Making use of the methods described in Example 1, e.g., for the preparation and analysis of cell lines expressing AR as a fluorescent target protein, this Example provides additional evidence that changes in protein interactions, e.g., changes in protein binding, as measured via changes in target movement, can support the identification of pharmacologically-relevant compounds. Specifically, known agonists and antagonists of AR were assayed as described in Example 1, except that the f_{bound} measured for AR was in the presence of an agonist at 25 nM, a potent antagonist at 10 μM, or the combination of agonist and antagonist at 25 nM and 10 μM, respectively.

While AR agonists were observed to increase f_{bound}, antagonists of AR were observed to cause a decrease in f_{bound} both in single treatment as well as when co-administered with the AR agonist (FIG. 24). Thus, this Example clearly indicates that both increases in f_{bound} and decreases in f_{bound} can be useful in identifying mechanistically distinct mechanisms for pharmacological interaction with the fluorescent target protein under observation.

### Example 3. htSMT Analysis of Target A Antagonists

Making use of the methods described in Example 1, e.g., for the preparation and analysis of cell lines expressing Target A as a fluorescent target protein, this Example provides additional evidence that changes in protein interactions, e.g., changes in protein-protein interactions in a signaling pathway unrelated to the ER signaling described in Example 1, can support the identification of pharmacologically-relevant compounds. In particular, FIG. 25 depicts the change in Target A movement in response to dose titrations of known Target A antagonists. Each series of shapes represents a different compound and error bars represent SEM. As evidenced by the shape of the curves, increasing concentrations of the antagonists induce measurable differences in median third quartile (Q3) jump length relative to DMSO, which is indicative of the liberation of Target A from a bound state in the presence of the antagonists.

### Example 4. htSMT Analysis of Competitive & Allosteric Antagonists

Making use of the methods described in Example 1, e.g., for the preparation and analysis of cell lines expressing exemplary receptor tyrosine kinases (Target B and Target C) and a helicase as fluorescent target proteins, this Example provides additional evidence that compounds impacting protein interactions, e.g., changes in protein-protein interactions in a signaling pathway, via competitive or allosteric inhibition, can support the identification of pharmacologically-relevant compounds. In particular, FIG. 26 depicts the change in Target B and Target C movements in the presence of competitive or allosteric antagonists. Each series of shapes represents a different compound normalized to DMSO and error bars represent SEM. As evidenced by the shape of the curves, increasing concentrations of the antagonists induce measurable differences in median third quartile (Q3) jump length relative to DMSO, which is indicative of Target B and Target C being differentially impacted by competitive and allosteric antagonists.

Similarly, FIG. 27 depicts the change in median Q3 jump length relative to DMSO as a function of time after compound addition for Target A, Target B and a helicase target. As evidence from the data presented, proteins treated with on-target inhibitors increase or decrease protein movement. In the case of Target A and Target B such changes occur within minutes post compound addition. In contrast, a helicase treated with either a pathway antagonist or an off-target modulator, such as the case with DNA damage induction, the change in protein movement takes several hours or more to reach maximal effect.

What is claimed is:

1. A method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
(i) illuminating a field of view in a sample plane disposed within the sample with a light beam to cause fluorescence by a subset of the target fluorescent proteins in the live cells; and
(ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and
(c) determining a change in the movement of the target fluorescent protein in the presence of the compound,
wherein the rate at which changes in the movement of the target fluorescent protein emerge is determined by comparing the changes in movement of the target fluorescent protein at the plurality of time points.

2. A method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
(i) illuminating a field of view in a sample plane disposed within the sample with a light beam to cause fluorescence by a subset of the target fluorescent proteins in the live cells, wherein the subset of the target fluorescent produces 100-100,000 molecular trajectories in a single detected field of view; and
(ii) detecting the fluorescence from a plurality of the target fluorescent proteins in the detected field of view of the sample plane via a detector device, wherein the detected field of view has a size of about 50 μm to less than 100 μm in a first dimension by about 50 μm to less than 100 μm in a second dimension; and
(c) determining a change in the movement of the target fluorescent protein in the presence of the compound,
wherein the rate at which changes in the movement of the target fluorescent protein emerge is determined by comparing the changes in movement of the target fluorescent protein at the plurality of time points.

3. A method determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
(i) illuminating a field of view in a sample plane disposed within the sample with a light beam to cause fluorescence by a subset of the target fluorescent proteins in the live cells; and
(ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and
(c) determining a change in the movement of target fluorescent proteins upon the addition of the compound, wherein the average change in movement of the target fluorescent proteins in the presence of the compound is at least 1%, at least 5%, at least 10% relative to the change observed in the absence of the compound,
wherein the rate at which changes in the movement of the target fluorescent protein emerge is determined by comparing the changes in movement of the target fluorescent protein at the plurality of time points.

4. A method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
(i) illuminating a field of view in a sample plane disposed within the sample with a light beam to cause fluorescence by a subset of the target fluorescent proteins in the live cells;
(ii) detecting the fluorescence from a plurality of the target fluorescent proteins in a detected field of view of the sample plane via a detector device, wherein the detected field of view has a size of about 50 μm to less than 100 μm in a first dimension by about 50 μm to less than 100 μm in a second dimension; and
(iii) detecting the fluorescence from a plurality of the target fluorescent proteins in the detected field of view of the sample plane at a rate of >100 detected FOVs per day, >10,000 FOVs per day, or >100,000 detected FOVs per day; and
(c) determining a change in the movement of target fluorescent proteins in the presence of the compound,
wherein the rate at which changes in the movement of the target fluorescent protein emerge is determined by comparing the changes in movement of the target fluorescent protein at the plurality of time points.

5. A method of determining the rate of emergence of a biological interaction between a compound and a target fluorescent protein in a live cell comprising:
(a) contacting a sample comprising a population of live cells with the compound, wherein the live cells comprise the target fluorescent protein;
(b) tracking the movement of a plurality of individual target fluorescent proteins in a plurality of live cells in the sample at a plurality of time points, wherein said tracking comprises:
(i) illuminating a field of view in a sample plane disposed within the sample with a light beam to cause fluorescence by a subset of the target fluorescent proteins in the live cells; and
(ii) detecting the fluorescence from a plurality of the target fluorescent proteins in the detected field of view of the sample plane via a detector device, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; and
(c) determining a change in the movement of the target fluorescent protein in the presence of the compound,
wherein the rate at which changes in the movement of the target fluorescent protein emerge is determined by comparing the changes in movement of the target fluorescent protein at the plurality of time points.

6. The method of any one of claims 1-5, wherein the change in movement detected is an increase in immobile trajectories indicating an increase in bound (f_{bound}) target fluorescent protein.

7. The method of any one of claims 1-5, wherein the change in movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(j) state occupation via inference.

8. The method of any one of claims 1-7, wherein the target fluorescent protein interacts in a larger molecular assembly.

9. The method of claim 8, wherein the target fluorescent protein is a ligand.

10. The method of claim 8, wherein the target fluorescent protein is a receptor.

11. The method of any one of claims 1-10, wherein the biological interaction is a direct interaction.

12. The method of claim 11, wherein the direct interaction comprises binding of the compound to the target fluorescent protein.

13. The method of any one of claims 1-10, where the biological interaction is an indirect interaction.

14. The method of claim 13, wherein the indirect interaction comprises the compound agonizing or antagonizing a larger molecular assembly comprising the target fluorescent protein.

15. A method of determining a dose of a compound, wherein said compound induces a change in the movement of a target fluorescent protein in a live cell comprising:
(a) contacting a plurality of samples with the compound,
(i) wherein each sample comprises a population of live cells;
(ii) wherein the live cells comprise the target fluorescent protein; and
(iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
(i) illuminating a field of view in a sample plane disposed within the sample with a light beam to cause fluorescence by at least a subset of the target fluorescent proteins in the live cells;
(ii) detecting the fluorescence from one or more of the target fluorescent proteins in the sample plane via a detector device, wherein the detected field of view has a size of about 50 μm to less than 100 μm in a first dimension by about 50 μm to less than 100 μm in a second dimension; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

16. A method of determining a dose of a compound that induces a change in the movement of a target fluorescent protein in a live cell comprising:
(a) contacting a plurality of samples with the compound,
(i) wherein each sample comprises a population of live cells;
(ii) wherein the live cells comprise the target fluorescent protein; and
(iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
(i) illuminating a field of view in a sample plane disposed within the sample with a light beam to cause fluorescence by at least a subset of the target fluorescent proteins in the live cells, wherein the subset of the target fluorescent proteins produces 100-100,000 molecular trajectories in a single detected field of view;
(ii) detecting the fluorescence from one or more of the target fluorescent proteins in the detected field of view of the sample plane via a detector device, wherein the detected field of view has a size of about 50 μm to less than 100 μm in a first dimension by about 50 μm to less than 100 μm in a second dimension; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

17. A method of determining a dose response of a compound that induces a change in the movement of a target fluorescent protein in a live cell comprising:
(a) contacting a plurality of samples with the compound,
(i) wherein each sample comprises a population of live cells;
(ii) wherein the live cells comprise the target fluorescent protein; and
(iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
(i) illuminating a field of view in a sample plane disposed within the sample with a light beam to cause fluorescence by at least a subset of the target fluorescent proteins in the live cells;
(ii) detecting the fluorescence from one or more of the target fluorescent proteins in a detected field of view of the sample plane via a detector device, wherein the detected field of view has a size of about 50 μm to less than 100 μm in a first dimension by about 50 μm to less than 100 μm in a second dimension; and
(c) determining the rate at which changes in the movement of the target fluorescent protein occur in the presence of the compound, wherein the average change in movement of the target fluorescent protein in the presence of the compound is at least 1%, at least 5%, at least 10%, relative to the change observed in the absence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

18. A method of determining a dose response of a compound that induces a change in the movement of a target fluorescent protein in a live cell comprising:
(a) contacting a plurality of samples with the compound,
(i) wherein each sample comprises a population of live cells;
(ii) wherein the live cells comprise the target fluorescent protein; and
(iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
(i) illuminating a field of view in a sample plane disposed within the sample with a light beam to cause fluorescence by at least a subset of the target fluorescent proteins in the cells;
(ii) detecting the fluorescence from one or more of the target fluorescent proteins in a detected field of view of the sample plane via a detector device, wherein the detected field of view has a size of about 50 μm to less than 100 μm in a first dimension by about 50 μm to less than 100 μm in a second dimension; and
iii) wherein said tracking comprises detecting the fluorescence from a plurality of the target fluorescent proteins in the detected field of view of the sample plane at a rate of >100 detected FOVs per day, >10,000 detected FOVs per day, or >100,000 detected FOVs per day; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

19. A method of determining a dose of a compound, wherein said compound induces a change in the movement of a target fluorescent protein in a live cell comprising:
(a) contacting a plurality of samples with the compound,
(i) wherein each sample comprises a population of live cells;
(ii) wherein the live cells comprise the target fluorescent protein; and
(iii) wherein the plurality of samples are contacted with distinct concentrations of the compound across a range of compound concentrations; and
(b) tracking the movement of individual target fluorescent proteins in a plurality of live cells of a sample at a plurality of time points, wherein said tracking comprises:
(i) illuminating a field of view in a sample plane disposed within the sample with a light beam to cause fluorescence by at least a subset of the target fluorescent proteins in the live cells;
(ii) detecting the fluorescence from one or more of the target fluorescent proteins in the sample plane via a detector device, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement; and
(c) determining the rate at which changes in the movement of the target fluorescent protein occur in the presence of the compound; and
(d) repeating steps (b)-(c) for each of the plurality of samples across the range of compound concentrations,
wherein the rate at which changes in the movement of the target fluorescent protein emerge is determined by comparing the changes in movement of the target fluorescent protein at the plurality of time points; and
wherein the dose is determined by said rate.

20. The method of any one of claims 15-19, wherein the change in movement detected is an increase in immobile trajectories indicating an increase in bound (f_{bound}) target fluorescent protein.

21. The method of any one of claims 15-19, wherein the change in movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(j) state occupation via inference.

22. The method of any one of claims 15-21, wherein the target fluorescent protein interacts in a larger molecular assembly.

23. The method of claim 22, wherein the target fluorescent protein is a ligand.

24. The method of claim 22, wherein the target fluorescent protein is a receptor.

25. The method of any one of claims 15-24, wherein the biological interaction is a direct interaction.

26. The method of claim 25, wherein the direct interaction comprises binding of the compound to the target fluorescent protein.

27. The method of any one of claims 15-24, where the biological interaction is an indirect interaction.

28. The method of claim 27, wherein the indirect interaction comprises the compound agonizing or antagonizing a larger molecular assembly comprising the target fluorescent protein.

29. A microscopy system configured to detect the rate of emergence of a biological interaction between a compound and a target fluorescent protein in a live cell comprising:
(a) a stage for supporting a sample, wherein the sample comprises a population of live cells, and where the live cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane and wherein the detected field of view has a size of about 50 μm to less than 100 μm in a first dimension by about 50 μm to less than 100 μm in a second dimension;
(e) a detector device for monitoring the light-based response from the target fluorescent proteins in the presence of the compound at a plurality of time points;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

30. A microscopy system configured to detect the rate of emergence of a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view in the sample plane, wherein the subset of the target fluorescent proteins produces 100-100,000 molecular trajectories in a single detected field of view and wherein the detected field of view has a size of about 50 μm to less than 100 μm in a first dimension by about 50 μm to less than 100 μm in a second dimension;
(d) a detector device for monitoring the light-based response from the target fluorescent proteins in the presence of the compound at a plurality of time points;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

31. A microscopy system configured to detect the rate of emergence of a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane and wherein the detected field of view has a size of about 50 μm to less than 100 μm in a first dimension by about 50 μm to less than 100 μm in a second dimension;
(d) a detector device for monitoring the light-based response from the target fluorescent proteins in the presence of the compound at a plurality of time points, wherein the average change in movement of the target fluorescent protein in the presence of the of the compound is at least 2%, at least 5%, at least 10%, relative to the change observed in the absence of the compound,
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

32. A microscopy system configured to detect the rate of emergence of a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a plurality of the target fluorescent proteins in the sample are disposed in a detected field of view of the sample plane and wherein the detected field of view has a size of about 50 μm to less than 100 μm in a first dimension by about 50 μm to less than 100 μm in a second dimension;
(d) a detector device for monitoring the light-based response from the target fluorescent proteins in the presence of the compound at a plurality of time points, wherein said monitoring comprises detecting the fluorescence from a plurality of the target fluorescent proteins in the detected field of view of the sample plane at a rate of >100 detected FOVs per day, >10,000 detected FOVs per day, >100,000 detected FOVs per day;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

33. A microscopy system configured to detect the rate of emergence of a biological interaction between a compound and a target fluorescent protein in a live cell comprising:
(a) a stage for supporting a sample, wherein the sample comprises a population of live cells, and where the live cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane, wherein the detected field of view has a size of about 50 μm to less than 100 μm in a first dimension by about 50 μm to less than 100 μm in a second dimension and wherein up to 70% of the detected field of view achieves sufficient laser illumination for tracking protein movement;
(e) a detector device for monitoring the light-based response from the target fluorescent proteins in the presence of the compound at a plurality of time points;
(e) a memory; and
(f) a processor in communication with the memory and the detector device, where the processor is capable of determining the change in the movement of the target fluorescent protein in the presence of the compound relative to the absence of the compound at a plurality of time points to thereby detect the rate of emergence of a biological interaction between a compound and a target fluorescent protein in a live cell.

34. The system of any one of claims 29-33, wherein the change in movement detected is an increase in immobile trajectories indicating an increase in bound (f_{bound}) target fluorescent protein.

35. The system of any one of claims 29-33, wherein the movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(j) state occupation via inference.

36. The system of any one of claims 29-35, wherein the target fluorescent protein interacts in a larger molecular assembly.

37. The system of claim 36, wherein the target fluorescent protein is a ligand.

38. The system of claim 36, wherein the target fluorescent protein is a receptor.

39. The system of any one of claims 29-38, wherein the biological interaction is a direct interaction.

40. The system of claim 39, wherein the direct interaction comprises binding of the compound to the target fluorescent protein.

41. The system of any one of claims 29-38, wherein the biological interaction is an indirect interaction.

42. The system of claim 41, wherein the indirect interaction comprises the compound agonizing or antagonizing a larger molecular assembly comprising the target fluorescent protein.
